(19) United States
(12) Patent Application Publication
(10) Pub. No.: US 2013/0053385 A1
(43) Pub. Date: Feb. 28, 2013

(54) METHOD OF COMBATING AND CONTROLLING PESTS

(71) Applicants: Stephane Andre Marie Jeanmart, Stein (CH); Russell Viner, Bracknell (GB); John Benjamin Taylor, Bracknell (GB); William Guy Whittingham, Bracknell (GB); Jeffrey Steven Wailes, Bracknell (GB); Sarah Margaret Targett, Bracknell (GB); Christopher John Mathews, Bracknell (GB); Michel Muehlebach, Bracknell (GB)

(72) Inventors: Stephane Andre Marie Jeanmart, Stein (CH); Russell Viner, Bracknell (GB); John Benjamin Taylor, Bracknell (GB); William Guy Whittingham, Bracknell (GB); Jeffrey Steven Wailes, Bracknell (GB); Sarah Margaret Targett, Bracknell (GB); Christopher John Mathews, Bracknell (GB); Michel Muehlebach, Bracknell (GB)

(73) Assignees: SYNGENTA LIMITED, Guildford, Surrey (GB); SYNGENTA CROP PROTECTION LLC, Greensboro, NC (US)

(21) Appl. No.: 13/517,046

(22) PCT Filed: Dec. 7, 2010
(86) PCT No.: PCT/EP10/69094

§ 371 Date: Nov. 14, 2012

(30) Foreign Application Priority Data
Dec. 18, 2009 (EP) 09180039.1

(51) Int. Cl. A01N 43/16; A01N 43/40; A01N 43/18; A01N 43/36; A01N 43/32; A01N 43/84; A01P 1/00; A01P 7/02; A01P 5/00; A01P 9/00; C07D 309/06; C07D 307/12; C07D 211/06; C07D 335/02; C07D 207/48; C07D 319/12; C07D 405/06; C07D 413/06; C07D 401/06; A01N 43/08

(52) U.S. Cl. 514/235.5; 514/451; 514/461; 514/330; 514/432; 514/424; 514/452; 514/326; 514/316; 549/427; 549/498; 546/226; 549/13; 548/542; 549/378; 546/214; 544/130; 546/189

(57) ABSTRACT

The use of a compound of formula I

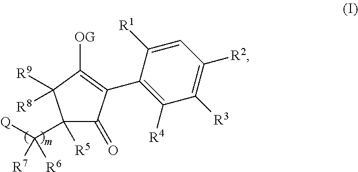

wherein the substituents are as defined in claim 1, or compositions containing them in controlling insects, acarines, nematodes or molluscs.

The present invention relates to methods of combating and controlling pests such as insects, acarines, nematodes or molluscs using cyclic dione compounds, and to pesticidal compositions comprising those compounds.

Cyclic dione compounds are described, for example, in WO01/74770 and WO96/03366.

It has now surprisingly been found that certain cyclic dione derivatives have good insecticidal properties.

The present invention therefore provides methods of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I):

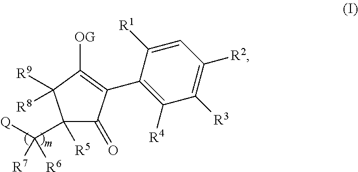

wherein
R¹ is hydrogen, methyl, ethyl, n-propyl, iso-propyl, halomethyl, haloethyl, halogen, vinyl, ethynyl, methoxy, ethoxy, halomethoxy, haloethoxy, cyclopropyl or halocyclopropyl,
R² and R³ are independently hydrogen, halogen, C₁-C₆alkyl, C₁-C₆haloalkyl, C₁-C₆alkoxy, C₁-C₆haloalkoxy, C₂-C₆alkenyl, C₂-C₆haloalkenyl, C₂-C₆alkynyl, C₂-C₆haloalkynyl, C₃-C₆alkenyloxy, C₃-C₆haloalkenyloxy, C₃-C₆alkynyloxy, C₃-C₆cycloalkyl, C₁-C₆alkylthio, C₁-C₆alkylsulfinyl, C₁-C₆alkylsulfonyl, C₁-C₆alkoxysulfonyl, C₁-C₆haloalkoxysulfonyl, cyano, nitro, phenyl, phenyl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, or heteroaryl or heteroaryl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl,
R⁴ is hydrogen, C₁-C₆alkyl, C₁-C₆haloalkyl, C₁-C₆alkoxy, C₁-C₆haloalkoxy, C₂-C₆alkenyl, C₂-C₆haloalkenyl, C₂-C₆alkynyl, C₃-C₆alkenyloxy, C₃-C₆haloalkenyloxy, C₃-C₆alkynyloxy, C₃-C₆cycloalkyl, C₁-C₆alkylthio, C₁-C₆alkylsulfinyl, C₁-C₆alkylsulfonyl, C₁-C₆alkoxysulfonyl, C₁-C₆haloalkoxysulfonyl or cyano,
R⁵, R⁶, R⁷, R⁸ and R⁹ are independently hydrogen, halogen, C₁-C₆alkyl, C₁-C₆haloalkyl, C₁-C₆alkoxy, C₁-C₆haloalkoxy, C₂-C₆alkenyl, C₂-C₆haloalkenyl, C₂-C₆alkynyl, C₃-C₆alkenyloxy, C₃-C₆haloalkenyloxy, C₃-C₆alkynyloxy, C₃-C₆cycloalkyl, C₁-C₆alkylthio, C₁-C₆alkylsulfinyl, C₁-C₆alkylsulfonyl, C₁-C₆haloalkylsulfonyl, C₁-C₆alkoxysulfonyl, C₁-C₆haloalkoxysulfonyl, cyano, nitro, phenyl, phenyl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, or heteroaryl or heteroaryl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl, C₁-C₃alkylsulfonyl, or benzyl or benzyl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, or C₃-C₆cycloalkylC₁-C₃alkyl in which a ring or chain methylene group is optionally replaced by an oxygen or sulfur atom or
R⁶ and R⁷ or R⁸ and R⁹ together with the carbon atoms to which they are attached form an optionally substituted 3- to 8-membered ring, optionally containing an oxygen, sulphur or nitrogen atom, or
R⁵ and R⁶ together form a bond,
Q is C₃-C₈ saturated or mono-unsaturated heterocyclyl containing at least one heteroatom selected from O, N and S, unsubstituted or substituted by a residue of formula ═O, ═N—R¹⁰ or C₁-C₄alkyl, C₁-C₄haloalkyl, C₁-C₄alkoxyC₁-C₂alkyl, C₃-C₆cycloalkyl, phenyl, phenyl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, where R¹⁰ is C₁-C₆alkyl, C₁-C₆haloalkyl, C₃-C₇cycloalkyl, C₁-C₆alkoxy, C₁-C₆haloalkoxy, C₁-C₆alkylsulfinyl, C₁-C₆alkylsulfonyl, C₁-C₆alkylcarbonyl, C₁-C₆haloalkylcarbonyl, C₁-C₆alkoxycarbonyl, C₁-C₆alkylaminocarbonyl, C₂-C₈dialkylaminocarbonyl, C₁-C₆haloalkylsulfinyl or C₁-C₆haloalkylsulfonyl,
m is 1, 2 or 3,
where R⁶ or R⁷ can have different meanings when m is 2 or 3, and
G is hydrogen or an agriculturally acceptable metal, sulfonium, ammonium or latentiating group,
or an agrochemically acceptable salt or an N-oxide thereof.

In the substituent definitions of the compounds of the formula I, each alkyl moiety either alone or as part of a larger group (such as alkoxy, alkylthio, alkylcarbonyl, alkylaminocarbonyl and dialkylaminocarbonyl is a straight or branched chain and is, for example, methyl, ethyl, n-propyl, n-butyl, n-pentyl, n-hexyl, isopropyl, n-butyl, sec-butyl, isobutyl, tert-butyl or neopentyl. The alkyl groups are suitably C₁-C₆ alkyl groups, but are preferably C₁-C₄ alkyl groups, and, more preferably, C₁-C₂alkyl groups. Alkenyl and alkynyl moieties can be in the form of straight or branched chains, and the alkenyl moieties, where appropriate, can be of either the (E)- or (Z)-configuration. Examples are vinyl, allyl and propargyl. Alkenyl and alkynyl moieties can contain one or more double and/or triple bonds in any combination. It is understood, that allenyl and alkylinylalkenyl are included in these terms.

Halogen is fluorine, chlorine, bromine or iodine.

Haloalkyl groups are alkyl groups which are substituted with one or more of the same or different halogen atoms and are, for example, CF₃, CF₂Cl, CF₂H, CCl₂H, FCH₂, ClCH₂, BrCH₂, CH₃CHF, (CH₃)₂CF, CF₃CH₂ or CHF₂CH₂.

The term “heteroaryl” preferably refers to an aromatic ring system containing at least one heteroatom and consisting either of a single ring or of two or more fused rings. Preferably, single rings will contain up to three and bicyclic systems up to four heteroatoms which will preferably be chosen from nitrogen, oxygen and sulphur. Examples of such groups include furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzisofuryl, benzothienyl, benzisothienyl, indolyl, isoindolyl, indazolyl, benzothiazolyl, benzisothiazolyl, benzoxazolyl, benzisoxazolyl, benzimidazolyl, 2,1,3-benzoxadiazole, quinolinyl, isoquinolinyl, cinnolinyl, phthalazinyl, quinazolinyl, quinoxalinyl, naphthyridinyl, benzotriazinyl, purinyl, pteridinyl and indolizinyl.

Preferred examples of heteroaromatic radicals include pyridyl, pyrimidinyl, triazinyl, thienyl, furyl, oxazolyl, isoxazolyl, 2,1,3-benzoxadiazolyl and thiazolyl.

Another group of preferred heteroaryls comprises furyl, thienyl, pyrazolyl, 1,2,3-triazolyl, 1,2,4-triazolyl, pyridyl, pyrimidinyl, pyridazinyl, pyrazinyl, quinolinyl, isoquinolinyl, cinnolinyl, quinazolinyl or quinoxalinyl.

The term “heterocyclyl” preferably refers to a non-aromatic, preferably monocyclic or bicyclic ring systems containing up to 8 atoms including at least one (preferably one or two) heteroatoms selected from O, S and N. Examples of such rings include 1,3-dithiane, 1,3-dioxane, 1,4-dioxane, morpholine, thiomorpholin, piperazine, tetrahydropyran, piperidine, thiane, 1,3-dioxolane, tetrahydrofuran, tetrahydrothiophene, pirolidine, imidazoline, azetidine, oxetane, thietane, aziridine, epoxide and thiirane.

Preferred examples of heterocyclic radicals include 1,3-dioxane, morpholine, thiomorpholin, tetrahydropyran, 1,3-dioxolane, tetrahydrofuran and tetrahydrothiophene

Cycloalkyl includes preferably cyclopropyl, cyclobutyl, cyclopentyl and cyclohexyl.

For substituted heterocyclyl groups such as the rings formed by R⁶ and R⁷, and R⁸ and R⁹, respectively, it is preferred that one or more substituents are independently selected from halogen, C₁-C₆alkyl, C₁-C₆haloalkyl, C₁-C₆alkoxy, C₁-C₆haloalkoxy, C₁-C₆alkylthio, C₁-C₆alkylsulfinyl, C₁-C₆alkylsulfonyl, nitro and cyano. It is to be understood that dialkylamino substituents include those where the dialkyl groups together with the N atom to which they are attached form a five, six or seven-membered heterocyclic ring which may contain one or two further heteroatoms selected from O, N or S and which is optionally substituted by one or two independently selected C₁-C₆alkyl groups. When heterocyclic rings are formed by joining two groups on an N atom, the resulting rings are suitably pyrrolidine, piperidine, thiomorpholine and morpholine each of which may be substituted by one or two independently selected C₁-C₆alkyl groups.

The invention relates also to the use of salts which the compounds of formula I are able to form with amines, alkali metal and alkaline earth metal bases or quaternary ammonium bases.

Among the alkali metal and alkaline earth metal hydroxides as salt formers, special mention should be made of the hydroxides of lithium, sodium, potassium, magnesium and calcium, but especially the hydroxides of sodium and potassium. The use of compounds of formula I according to the invention also includes the use of hydrates which may be formed during the salt formation.

Examples of amines suitable for ammonium salt formation include ammonia as well as primary, secondary and tertiary C₁-C₁₈alkylamines, C₁-C₄hydroxyalkylamines and C₂-C₄-alkoxyalkylamines, for example methylamine, ethylamine, n-propylamine, isopropylamine, the four butylamine isomers, n-amylamine, isoamylamine, hexylamine, heptylamine, octyl-amine, nonylamine, decylamine, pentadecylamine, hexadecylamine, heptadecylamine, octadecylamine, methylethylamine, methylisopropylamine, methylhexylamine, methyl-nonylamine, methylpentadecylamine, methyloctadecylamine, ethylbutylamine, ethylheptyl-amine, ethyloctylamine, hexylheptylamine, hexyloctylamine, dimethylamine, diethylamine, di-n-propylamine, diisopropylamine, di-n-butylamine, di-n-amylamine, diisoamylamine, dihexyl-amine, diheptylamine, dioctylamine, ethanolamine, n-propanolamine, isopropanolamine, N,N-diethanolamine, N-ethylpropanolamine, N-butylethanolamine, allylamine, n-but-2-enyl-amine, n-pent-2-enylamine, 2,3-dimethylbut-2-enylamine, dibut-2-enylamine, n-hex-2-enyl-amine, propylenediamine, trimethylamine, triethylamine, tri-n-propylamine, triisopropylamine, tri-n-butylamine, triisobutylamine, tri-sec-butylamine, tri-n-amylamine, methoxyethylamine and ethoxyethylamine; heterocyclic amines, for example pyridine, quinoline, isoquinoline, morpholine, piperidine, pyrrolidine, indoline, quinuclidine and azepine; primary arylamines, for example anilines, methoxyanilines, ethoxyanilines, o-, m- and p-toluidines, phenylene-diamines, benzidines, naphthylamines and o-, m- and p-chloroanilines; but especially triethyl-amine, isopropylamine and diisopropylamine.

Preferred quaternary ammonium bases suitable for salt formation correspond, for example, to the formula [N(Rₐ R_{b} R_{c} R_{d})]OH wherein Rₐ, R_{b}, R_{c} and R_{d} are each independently of the others C₁-C₄alkyl. Further suitable tetraalkylammonium bases with other anions can be obtained, for example, by anion exchange reactions.

Agriculturally acceptable metals are alkali metal or alkaline earth metal ions, for example sodium, potassium, magnesium and calcium ions, and transition metal ions, for example copper and iron atoms. Suitable ammonium ions are NH₄⁺, alkylammonium, dialkylammonium, triakylammonium and tetraalkylammonium ions. Suitable sulfonium ions are trialkylsulfonium ions, for example trimethylsulfonium ions.

It should be understood that in those compounds of formula I, where G is a metal, ammonium or sulfonium as mentioned above and as such represents a cation, the corresponding negative charge is largely delocalised across the O—C═C—C═O unit.

The latentiating groups G are selected to allow its removal by one or a combination of biochemical, chemical or physical processes to afford compounds of formula I where G is H before, during or following application to the treated area or plants. Examples of these processes include enzymatic cleavage, chemical hydrolysis and photoloysis. Compounds bearing such groups G may offer certain advantages, such as improved penetration of the cuticula of the plants treated, increased tolerance of crops, improved compatibility or stability in formulated mixtures containing other herbicides, herbicide safeners, plant growth regulators, fungicides or insecticides, or reduced leaching in soils.

The latentiating group G is preferably selected from the groups C₁-C₈ alkyl, C₂-C₈ haloalkyl, phenylC₁-C₈alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), heteroarylC₁-C₈alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), C₃-C₈ alkenyl, C₃-C₈ haloalkenyl, C₃-C₈ alkynyl, C(X^{a})—R^{a}, C(X^{b})—X^{c}—R^{b}, C(X^{d})—N(R^{c})—R^{d}, —SO₂—R^{e}, —P(X^{e})(R^{f})—R^{g} or CH₂—X^{f}—R^{h} wherein X^{a}, X^{b}, X^{c}, X^{d}, X^{e} and X^{f} are independently of each other oxygen or sulfur;

R^{a} is H, C₁-C₁₈alkyl, C₂-C₁₈alkenyl, C₂-C₁₈alkynyl, C₁-C₁₀haloalkyl, C₁-C₁₀cyanoalkyl, C₁-C₁₀nitroalkyl, C₁-C₁₀aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynylC₁-C₅oxyalkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₁-C₅alkylaminoC₁-C₅alkyl, C₃-C₆-trialkylsilylC₁-C₅alkyl, phenylC₁-C₅alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl, (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₂-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃ alkyl, C₁-C₃haloalkyl, C₁-C₃alkoxy, C₁-C₃haloalkoxy, halogen, cyano or nitro,
R^{b} is C₁-C₁₈alkyl, C₃-C₁₈alkenyl, C₃-C₁₈alkynyl, C₂-C₁₀haloalkyl, C₁-C₁₀cyanoalkyl, C₁-C₁₀nitroalkyl, C₂-C₁₀aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynyloxyC₁-C₅alkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₁-C₅alkylaminoC₁-C₅alkyl, C₃-C₆-trialkylsilylC₁-C₅alkyl, phenylC₁-C₅alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl, (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₃-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃ alkyl, C₁-C₃haloalkyl, C₁-C₃alkoxy, C₁-C₃haloalkoxy, halogen, cyano or nitro,
R^{c} and R^{d} are each independently of each other hydrogen, C₁-C₁₀alkyl, C₃-C₁₀alkenyl, C₃-C₁₀alkynyl, C₂-C₁₀haloalkyl, C₁-C₁₀cyanoalkyl, C₁-C₁₀nitroalkyl, C₁-C₁₀aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynyloxyC₁-C₅alkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₂-C₅alkylaminoalkyl, C₃-C₆-trialkylsilylC₁-C₅alkyl, phenylC₁-C₅alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl, (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₂-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃ alkyl, C₁-C₃haloalkyl, C₁-C₃alkoxy, C₁-C₃haloalkoxy, halogen, cyano or nitro, heteroarylamino or heteroarylamino substituted by C₁-C₃ alkyl, C₁-C₃haloalkyl, C₁-C₃alkoxy, C₁-C₃haloalkoxy, halogen, cyano or nitro, diheteroarylamino or diheteroarylamino substituted by C₁-C₃ alkyl, C₁-C₃haloalkyl, C₁-C₃alkoxy, C₁-C₃haloalkoxy, halogen, cyano or nitro, phenylamino or phenylamino substituted by C₁-C₃alkyl, C₁-C₃haloalkyl, C₁-C₃alkoxy, C₁-C₃haloalkoxy, halogen, cyano or by nitro, diphenylamino or diphenylamino substituted by C₁-C₃alkyl, C₁-C₃haloalkyl, C₁-C₃alkoxy, C₁-C₃haloalkoxy, halogen, cyano or by nitro or C₃-C₇cycloalkylamino, di-C₃-C₇cycloalkylamino or C₃-C₇cycloalkoxy or R^{c} and R^{d} may join together to form a 3-7 membered ring, optionally containing one heteroatom selected from O or S,
R^{e} is C₁-C₁₀alkyl, C₂-C₁₀alkenyl, C₂-C₁₀alkynyl, C₁-C₁₀haloalkyl, C₁-C₁₀cyanoalkyl, C₁-C₁₀nitroalkyl, C₁-C₁₀aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynyloxyC₁-C₅alkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₁-C₅alkylaminoC₁-C₅alkyl, C₃-C₆-trialkylsilylC₁-C₅alkyl, phenylC₁-C₅alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₂-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃ alkyl, C₁-C₃haloalkyl, C₁-C₃alkoxy, C₁-C₃haloalkoxy, halogen, cyano or by nitro, heteroarylamino or heteroarylamino substituted by C₁-C₃ alkyl, C₁-C₃haloalkyl, C₁-C₃alkoxy, C₁-C₃haloalkoxy, halogen, cyano or by nitro, diheteroarylamino or diheteroarylamino substituted by C₁-C₃ alkyl, C₁-C₃haloalkyl, C₁-C₃alkoxy, C₁-C₃haloalkoxy, halogen, cyano or nitro, phenylamino or phenylamino substituted by C₁-C₃alkyl, C₁-C₃haloalkyl, C₁-C₃alkoxy, C₁-C₃haloalkoxy, halogen, cyano or nitro, diphenylamino, or diphenylamino substituted by C₁-C₃alkyl, C₁-C₃haloalkyl, C₁-C₃alkoxy, C₁-C₃haloalkoxy, halogen, cyano or nitro, or C₃-C₇cycloalkylamino, diC₃-C₇cycloalkylamino or C₃-C₇cycloalkoxy, C₁-C₁₀alkoxy, C₁-C₁₀haloalkoxy, C₁-C₅alkylamino or C₂-C₈dialkylamino,
R^{f} and R^{g} are each independently of each other C₁-C₁₀alkyl, C₂-C₁₀alkenyl, C₂-C₁₀alkynyl, C₁-C₁₀alkoxy, C₁-C₁₀haloalkyl, C₁-C₁₀cyanoalkyl, C₁-C₁₀nitroalkyl, C₁-C₁₀aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynyloxyC₁-C₅alkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₂-C₅alkylaminoalkyl, C₃-C₈-trialkylsilylC₁-C₅alkyl, phenylC₁-C₅alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₂-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃ alkyl, C₁-C₃haloalkyl, C₁-C₃alkoxy, C₁-C₃haloalkoxy, halogen, cyano or by nitro, heteroarylamino or heteroarylamino substituted by C₁-C₃ alkyl, C₁-C₃haloalkyl, C₁-C₃alkoxy, C₁-C₃haloalkoxy, halogen, cyano or by nitro, diheteroarylamino or diheteroarylamino substituted by C₁-C₃ alkyl, C₁-C₃haloalkyl, C₁-C₃alkoxy, C₁-C₃haloalkoxy, halogen, cyano or nitro, phenylamino or phenylamino substituted by C₁-C₃alkyl, C₁-C₃haloalkyl, C₁-C₃alkoxy, C₁-C₃haloalkoxy, halogen, cyano or nitro, diphenylamino, or diphenylamino substituted by C₁-C₃alkyl, C₁-C₃haloalkyl, C₁-C₃alkoxy, C₁-C₃haloalkoxy, halogen, cyano or nitro, or C₃-C₇cycloalkylamino, diC₃-C₇cycloalkylamino or C₃-C₇cycloalkoxy, C₁-C₁₀haloalkoxy, C₁-C₅alkylamino or C₂-C₈dialkylamino, benzyloxy or phenoxy, wherein the benzyl and phenyl groups may in turn be substituted by C₁-C₃alkyl, C₁-C₃haloalkyl, C₁-C₃alkoxy, C₁-C₃haloalkoxy, halogen, cyano or nitro, and
R^{h} is C₁-C₁₀alkyl, C₃-C₁₀alkenyl, C₃-C₁₀alkynyl, C₁-C₁₀haloalkyl, C₁-C₁₀cyanoalkyl, C₁-C₁₀nitroalkyl, C₂-C₁₀aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynyloxyC₁-C₅alkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₁-C₅alkylaminoC₁-C₅alkyl, C₃-C₆-trialkylsilylC₁-C₅alkyl, phenylC₁-C₅alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), heteroarylC₁-C₅alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), phenoxyC₁-C₅alkyl (wherein wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), heteroaryloxyC₁-C₅alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), C₃-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen or by nitro, or heteroaryl, or heteroaryl substituted by C₁-C₃alkyl, C₁-C₃haloalkyl, C₁-C₃alkoxy, C₁-C₃haloalkoxy, halogen, cyano or by nitro.

In particular, the latentiating group G is a group —C(X^{a})—R^{a} or —C(X^{b})—X^{c}—R^{b}, and the meanings of X^{a}, R^{a}, X^{b}, X^{c} and R^{b} are as defined above.

It is preferred that G is hydrogen, an alkali metal or alkaline earth metal, where hydrogen is especially preferred.

Depending on the nature of the substituents, compounds of formula (I) may exist in different isomeric forms. When G is hydrogen, for example, compounds of formula (I) may exist in different tautomeric forms:

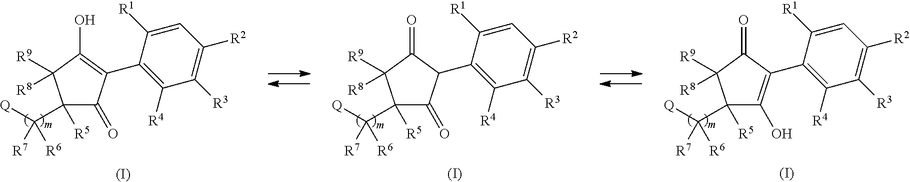

In a preferred group of compounds of the formula (I) for use in the invention, R¹ is methyl, ethyl or methoxy.

Preferably, R² and R³ are independently hydrogen, halogen, C₁-C₆alkyl, C₁-C₆alkoxy, C₂-C₆alkenyl, C₂-C₆alkynyl, C₂-C₆haloalkynyl, phenyl or phenyl substituted by C₁-C₄alkyl, C₁-C₃haloalkyl, cyano, nitro, halogen or C₁-C₃alkylsulfonyl, and, more preferably, R² and R³ are independently hydrogen, chlorine, bromine, methyl, methoxy, ethyl, ethoxy, ethenyl, ethynyl, phenyl or phenyl substituted by methyl, trifluoromethyl, cyano, nitro, fluorine, chlorine or methylsulfonyl.

In another group of preferred compounds of formula (I) for use in the invention, R² and R³ are independently thienyl, thienyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, furyl, furyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, pyrazolyl, pyrazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, thiazolyl, thiazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, oxazolyl, oxazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, isothiazolyl, isothiazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, isoxazolyl, isoxazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, triazolyl, triazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, oxadiazolyl, oxadiazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, thiadiazolyl, thiadiazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, tetrazolyl, tetrazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, pyridyl, pyridyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, pyrimidinyl, pyrimidinyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, pyridazinyl, pyridazinyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, pyrazinyl or pyrazinyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, triazinyl or triazinyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl.

Preferably, R³ is hydrogen.

Preferably, R⁴ is hydrogen, methyl, ethyl, n-propyl, iso-propyl, halomethyl, haloethyl, halogen, vinyl, ethynyl, methoxy, ethoxy, halomethoxy or haloethoxy, and more preferably R⁴ is hydrogen, methyl, ethyl, chlorine, bromine, ethenyl, ethynyl, methoxy or ethoxy.

Preferably, R¹, R² and R⁴ are methyl and R³ is hydrogen.

In another preferred group of the compounds of the formula (I) for use in the invention, R⁵ is hydrogen, halogen, C₁-C₆alkyl, C₁-C₆haloalkyl, C₁-C₆alkoxy or C₁-C₆haloalkoxy, and, more preferably, R⁵ is hydrogen or methyl.

Preferably, in the compounds of the formula (I) for use in the invention, R⁶ and R⁷ independently are hydrogen, halogen, C₁-C₆alkyl, C₁-C₆haloalkyl, C₁-C₆alkoxy or C₁-C₆haloalkoxy, and, more preferably, R⁶ and R⁷ independently are hydrogen or methyl.

In another preferred group of compounds of the formula (I) for use in the invention, R⁸ and R⁹ independently are hydrogen, halogen, C₁-C₆alkyl, C₁-C₆haloalkyl, C₁-C₆alkoxy or C₁-C₆haloalkoxy, and, more preferably, R⁸ and R⁹ independently are hydrogen or methyl.

Preferred saturated or mono-unsaturated rings Q are those of the formula

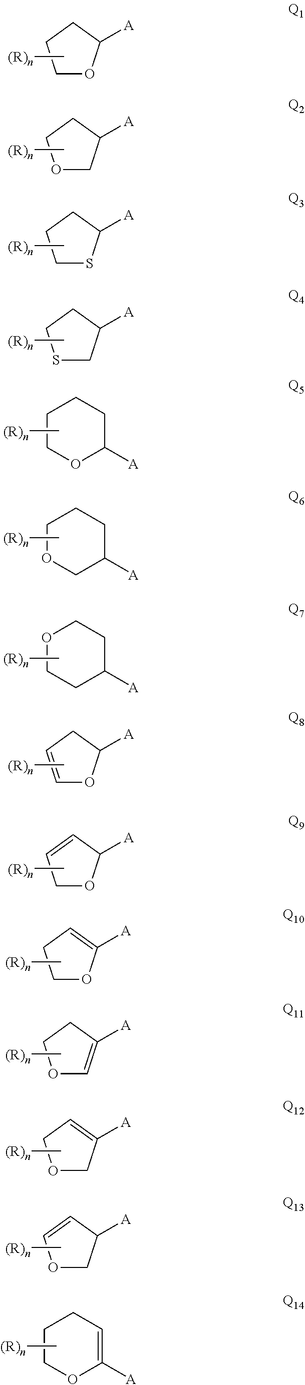

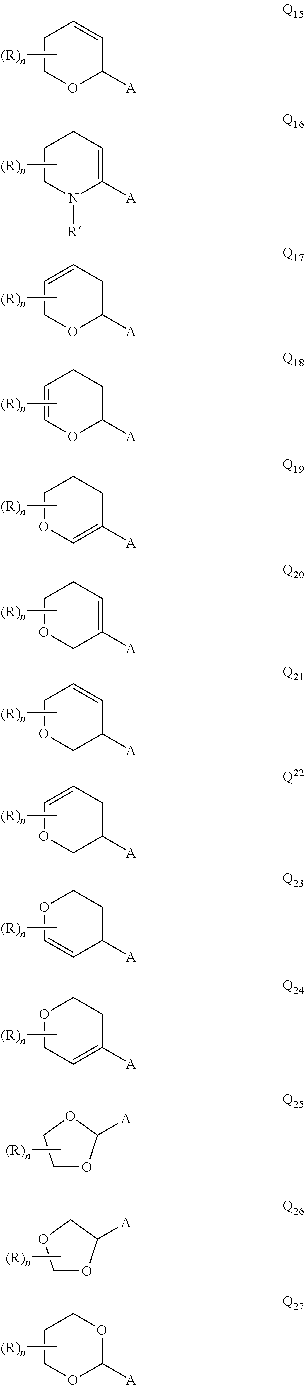

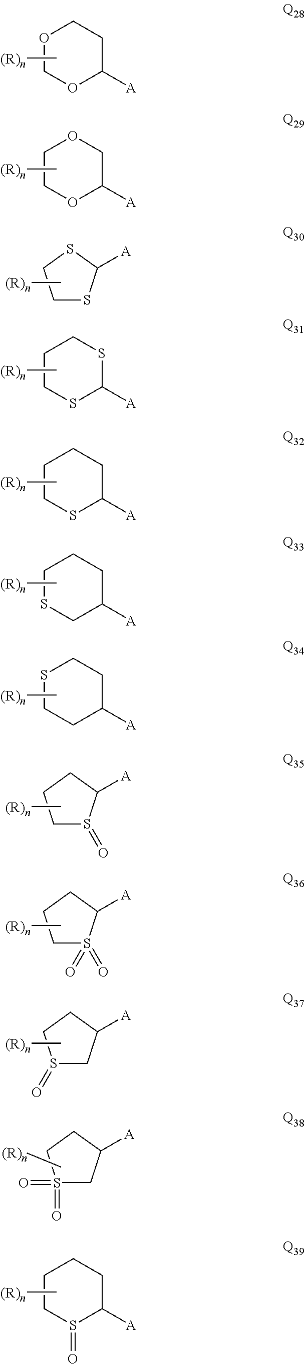

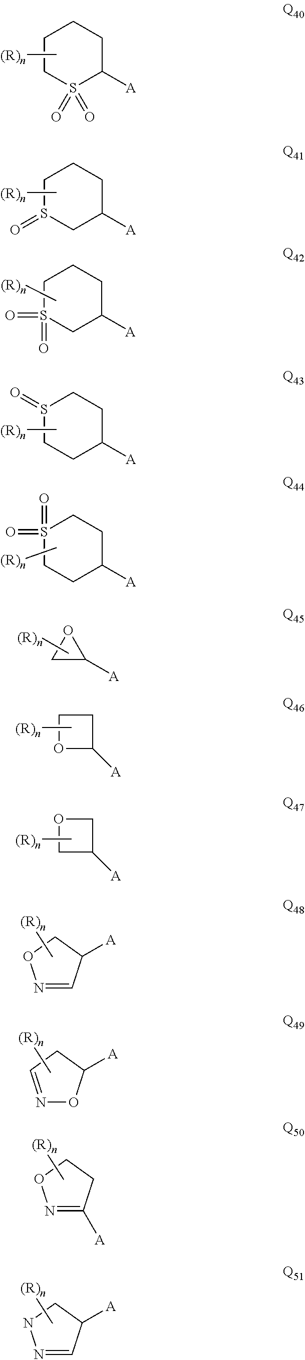

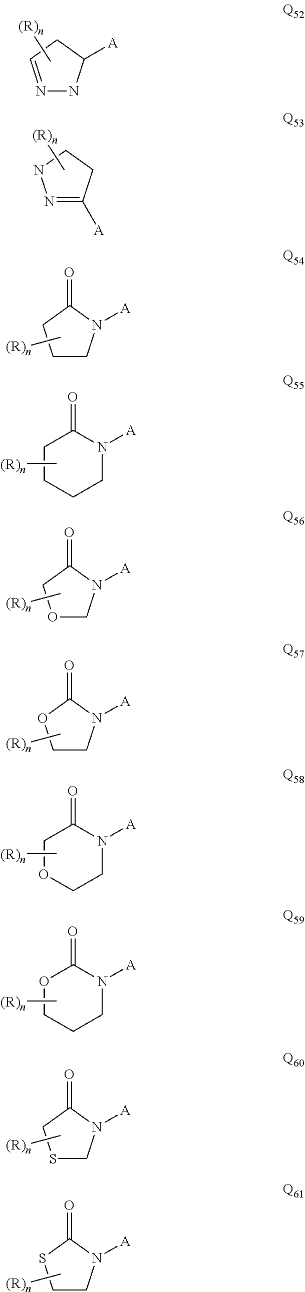

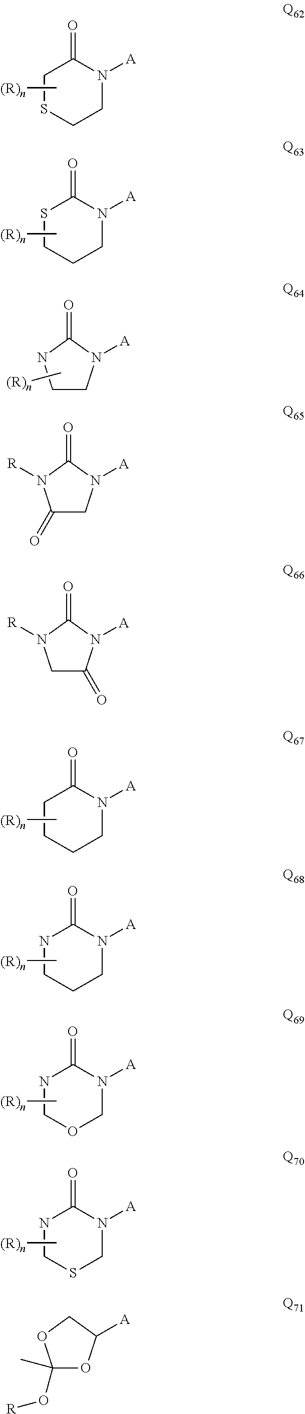

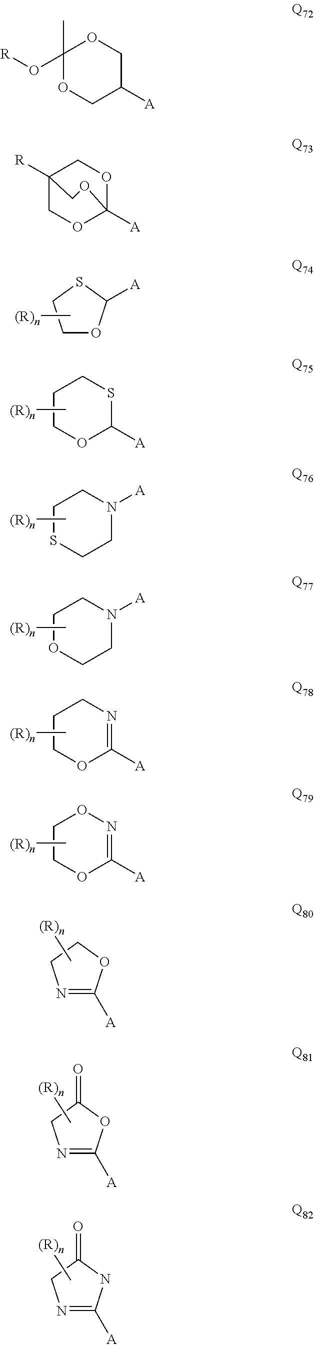

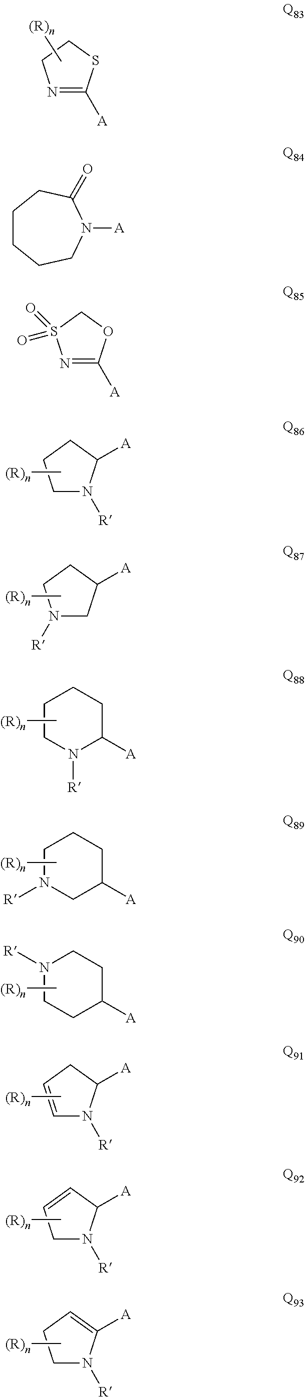

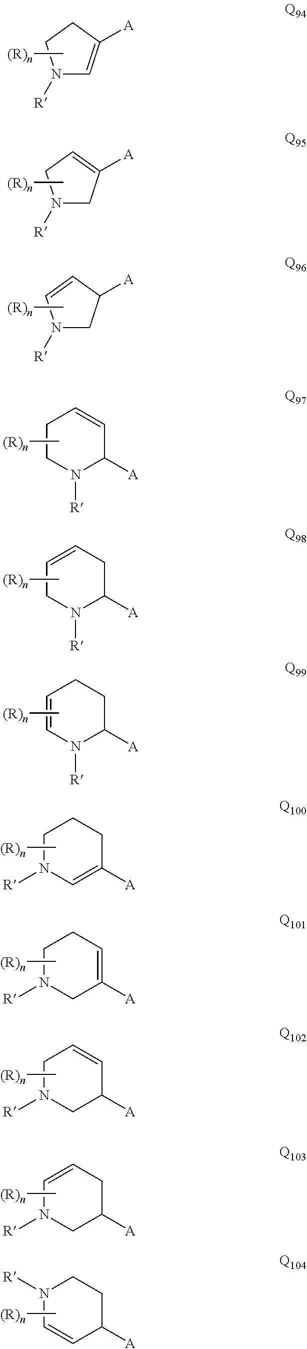

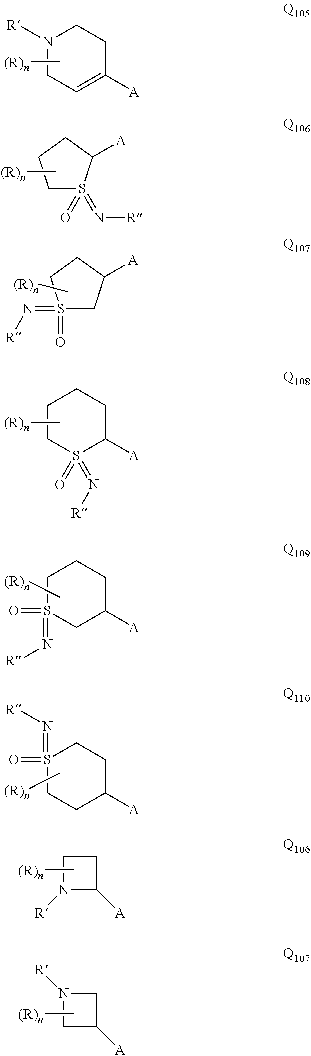

wherein
R is hydrogen, halogen, C₁-C₆alkyl, C₁-C₆haloalkyl, C₁-C₆alkoxy, C₁-C₆haloalkoxy, C₂-C₆alkenyl, C₂-C₆haloalkenyl, C₂-C₆alkynyl, C₂-C₆haloalkynyl, C₃-C₆alkenyloxy, C₃-C₆haloalkenyloxy, C₃-C₆alkynyloxy, C₃-C₆cycloalkyl, C₁-C₆alkylthio, C₁-C₆alkylsulfinyl, C₁-C₆alkylsulfonyl, C₁-C₆alkoxysulfonyl, C₁-C₆haloalkoxysulfonyl, cyano, nitro, phenyl, phenyl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, or heteroaryl or heteroaryl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl,
R′ is hydrogen, C₁-C₆alkyl, C₁-C₆haloalkyl, C₃-C₇cycloalkyl, C₁-C₆alkoxy, C₁-C₆haloalkoxy, C₁-C₆alkylsulfinyl, C₁-C₆alkylsulfonyl, C₁-C₆alkylcarbonyl, C₁-C₆haloalkylcarbonyl, C₁-C₆alkoxycarbonyl, C₁-C₆alkylaminocarbonyl, C₂-C₈dialkylaminocarbonyl, C₆-C₁₀arylsulfonyl, C₆-C₁₀arylcarbonyl, C₆-C₁₀arylaminocarbonyl, C₇-C₁₆arylalkylaminocarbonyl, C₁-C₉hetarylsulfonyl, C₁-C₉hetarylcarbonyl, C₁-C₉hetarylaminocarbonyl, C₂-C₁₅hetarylalkylaminocarbonyl,
R″ is hydrogen, C₁-C₆alkyl, C₁-C₆haloalkyl, C₃-C₇cycloalkyl, C₁-C₆alkoxy, C₁-C₆haloalkoxy, C₁-C₆alkylsulfinyl, C₁-C₆alkylsulfonyl, C₁-C₆alkylcarbonyl, C₁-C₆haloalkylcarbonyl, C₁-C₆alkoxycarbonyl, C₁-C₆alkylaminocarbonyl, C₂-C₈dialkylaminocarbonyl, C₁-C₆haloalkylsulfinyl or C₁-C₆haloalkylsulfonyl,
n is 0, 1, 2, 3 or 4 and
A denotes the position of attachment to the —(CR⁶R⁷)ₘ— moiety.

Groups Q₁, Q₂, Q₃, Q₄, Q₅, Q₆, Q₇, Q₂₅, Q₂₆, Q₂₇, Q₂₈, Q₂₉, Q₈₆, Q₈₇, Q₈₈, Q₈₉, Q₉₀ are more preferred, and groups Q₁ to Q₇ are particularly preferred.

Preferably, R and R′ are independently hydrogen, C₁-C₄alkyl, C₁-C₄haloalkyl, C₁-C₄alkoxy or C₁-C₄haloalkoxy, and R″ is hydrogen, C₁-C₄alkyl, C₁-C₄haloalkyl, C₁-C₄alkoxy, C₁-C₄haloalkoxy or C₁-C₆haloalkylcarbonyl.

Preferably, n is 0, 1 and 2.

Preferably, in the compounds of the formula (I) for use in the invention, m is 1 or 2 and most preferably m is 1.

Certain compounds of formula (I) are alkenes, and as such undergo further reactions typical of alkenes to give additional compounds of formula (I) according to known procedures. Example of such reaction include, but are not restricted to, halogenation or hydrogenation

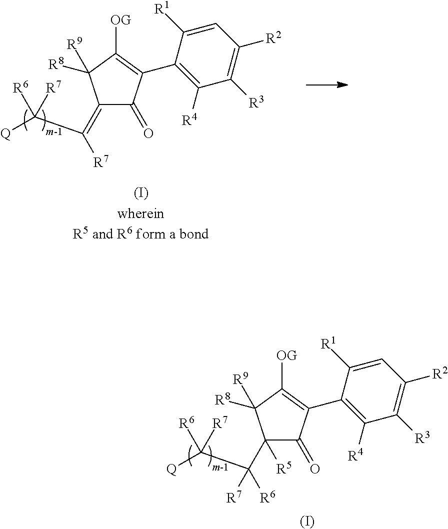

Compounds of formula (I) wherein R⁵ and R⁶ form a bond and R⁷ is halogen (preferably chloride or bromide) or R⁷ is C₁-C₆alkylsulfonate (preferably mesylate) or C₁-C₆haloalkylsulfonate (preferably triflate) or an arylsulfonate (preferable tosylate) may undergo a cross-coupling reaction with a suitable coupling partner under conditions described in the literature for Suzuki-Miyaura, Sonogashira and related cross-coupling reactions to give additional compounds of formula (I) (see, for example, O'Brien, C. J. and Organ, M. G. Angew. Chem. Int. Ed. (2007), 46, 2768-2813; Suzuki, A. Journal of Organometallic Chemistry (2002), 653, 83; Miyaura N. and Suzuki, A. Chem. Rev. (1995), 95, 2457-2483).

Those skilled in the art will appreciate that compounds of formula (I) may contain a aromatic moiety bearing one or more substituents capable of being transformed into alternative substituents under known conditions, and that these compounds may themselves serve as intermediates in the preparation of additional compounds of formula (I).

For example, compounds of formula (I) wherein R¹, R², R³ or R⁴ is alkenyl or alkynyl, may be reduced to compounds of formula (I) wherein R¹, R², R³ or R⁴ is alkyl under known conditions and compounds of formula (I) wherein R¹, R², R³ or R⁴ is halogen, preferably bromide or iodine, may undergo a cross-coupling reaction with a suitable coupling partner under conditions described in the literature for Suzuki-Miyaura, Sonogashira and related cross-coupling reactions to give additional compounds of formula (I) (see, for example, O'Brien, C. J. and Organ, M. G. Angew. Chem. Int. Ed. (2007), 46, 2768-2813; Suzuki, A. Journal of Organometallic Chemistry (2002), 653, 83; Miyaura N. and Suzuki, A. Chem. Rev. (1995), 95, 2457-2483).

Compounds of formula (I) wherein G is C₁-C₈alkyl, C₂-C₈haloalkyl, phenylC₁-C₈alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsufinyl, C₁-C₃alkylsulfonyl, halogen, cyano or by nitro), heteroarylC₁-C₈alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsufinyl, C₁-C₃alkylsulfonyl, halogen, cyano or by nitro), C₃-C₈ alkenyl, C₃-C₈haloalkenyl, C₃-C₈alkynyl, C(X^{a})—R^{a}, C(X^{b})—X^{c}—R^{b}, C(X^{d})—N(R^{c})—R^{d}, —SO₂—R^{e}, —P(X^{e})(R^{f})—R^{g} or CH₂—X^{f}—R^{h} where X^{a}, X^{b}, X^{c}, X^{d}, X^{e}, X^{f}, R^{a}, R^{b}, R^{c}, R^{d}, R^{e}, R^{f}, R^{g} and R^{h} are as defined above may be prepared by treating compounds of formula (A), which are compounds of formula (I) wherein G is H, with a reagent G-Z, wherein G-Z is alkylating agent such as an alkyl halide (the definition of alkyl halides includes simple C₁-C₈alkyl halides such as methyl iodide and ethyl iodide, substituted alkyl halides such as chloromethyl alkyl ethers, Cl—CH₂—X^{f}—R^{h}, wherein X^{f} is oxygen, and chloromethyl alkyl sulfides Cl—CH₂—X^{f}—R^{h}, wherein X^{f} is sulfur), a C₁-C₈alkyl sulfonate, or a di-C₁-C₈alkyl sulfate, or with a C₃-C₈alkenyl halide, or with a C₃-C₈alkynyl halide, or with an acylating agent such as a carboxylic acid, HO—C(X^{a})R^{a}, wherein X^{a} is oxygen, an acid chloride, Cl—C(X^{a})R^{a}, wherein X^{a} is oxygen, or acid anhydride, [R^{a}C(X^{a})]₂O, wherein X^{a} is oxygen, or an isocyanate, R^{c}N═C═O, or a carbamoyl chloride, Cl—C(X^{d})—N(R^{c})—R^{d} (wherein X^{d} is oxygen and with the proviso that neither R^{c} nor R^{d} is hydrogen), or a thiocarbamoyl chloride Cl—C(X^{d})—N(R^{c})—R^{d} (wherein X^{d} is sulfur and with the proviso that neither R^{c} nor R^{d} is hydrogen) or a chloroformate, Cl—C(X^{b})—X^{c}—R^{b}, (wherein X^{b} and X^{c} are oxygen), or a chlorothioformate Cl—C(X^{b})—X^{c}—R^{b} (wherein X^{b} is oxygen and X^{c} is sulfur), or a chlorodithioformate Cl—C(X^{b})—X^{c}—R^{b}, (wherein X^{b} and X^{c} are sulfur), or an isothiocyanate, R^{c}N═C═S, or by sequential treatment with carbon disulfide and an alkylating agent, or with a phosphorylating agent such as a phosphoryl chloride, Cl—P(X^{e})(R^{f})—R^{g} or with a sulfonylating agent such as a sulfonyl chloride Cl—SO₂—R^{e}, preferably in the presence of at least one equivalent of base.

Isomeric compounds of formula (I) may be formed. For example, compounds of formula (A) may give rise to two isomeric compounds of formula (I), or to isomeric mixtures of compounds of formula (I). This invention covers both isomeric compounds of formula (I), together with mixtures of these compounds in any ratio.

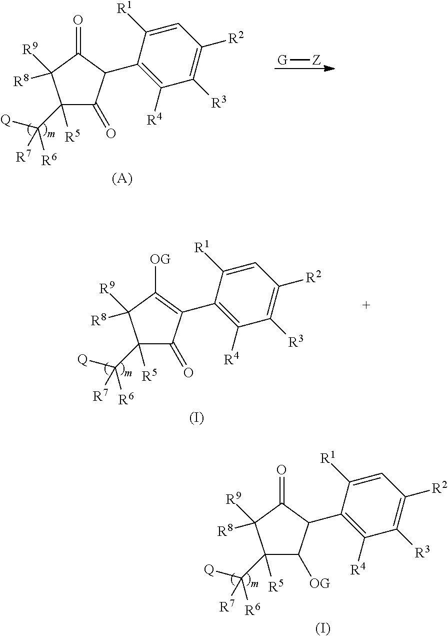

The O-alkylation of cyclic 1,3-diones is known; suitable methods are described, for example, in U.S. Pat. No. 4,436,666. Alternative procedures have been reported by Pizzorno, M. T. and Albonico, S. M. Chem. Ind. (London) (1972), 425; Born, H. et al. J. Chem. Soc. (1953), 1779; Constantino, M. G. et al. Synth. Commun. (1992), 22 (19), 2859; Tian, Y. et al. Synth. Commun. (1997), 27 (9), 1577; Chandra Roy, S. et al., Chem. Lett. (2006), 35 (1), 16; Zubaidha, P. K. et al. Tetrahedron Lett. (2004), 45, 7187 and by Zwanenburg, B. et al. Tetrahedron (2005), 45 (22), 7109.

The acylation of cyclic 1,3-diones may be effected by procedures similar to those described, for example, in U.S. Pat. No. 4,551,547, U.S. Pat. No. 4,175,135, U.S. Pat. No. 4,422,870, U.S. Pat. No. 4,659,372 and U.S. Pat. No. 4,436,666. Typically diones of formula (A) may be treated with the acylating agent in the presence of at least one equivalent of a suitable base, optionally in the presence of a suitable solvent. The base may be inorganic, such as an alkali metal carbonate or hydroxide, or a metal hydride, or an organic base such as a tertiary amine or metal alkoxide. Examples of suitable inorganic bases include sodium carbonate, sodium or potassium hydroxide, sodium hydride, and suitable organic bases include trialkylamines, such as trimethylamine and triethylamine, pyridines or other amine bases such as 1,4-diazobicyclo[2.2.2]octane and 1,8-diazabicyclo[5.4.0]undec-7-ene. Preferred bases include triethylamine and pyridine. Suitable solvents for this reaction are selected to be compatible with the reagents and include ethers such as tetrahydrofuran and 1,2-dimethoxyethane and halogenated solvents such as dichloromethane and chloroform. Certain bases, such as pyridine and triethylamine, may be employed successfully as both base and solvent. For cases where the acylating agent is a carboxylic acid, acylation is preferably effected in the presence of a coupling agent such as 2-chloro-1-methylpyridinium iodide, N,N-dicyclohexylcarbodiimide, 1-(3-dimethylaminopropyl)-3-ethylcarbodiimide and N,N′-carbodiimidazole, and optionally a base such as triethylamine or pyridine in a suitable solvent such as tetrahydrofuran, dichloromethane or acetonitrile. Suitable procedures are described, for example, by Zhang, W. and Pugh, G. Tetrahedron Lett. (1999), 40 (43), 7595 and Isobe, T. and Ishikawa, T. J. Org. Chem. (1999), 64 (19) 6984.

Phosphorylation of cyclic-1,3-diones may be effected using a phosphoryl halide or thiophosphoryl halide and a base by procedures analogous to those described in U.S. Pat. No. 4,409,153.

Sulfonylation of compounds of formula (A) may be achieved using an alkyl or aryl sulfonyl halide, preferably in the presence of at least one equivalent of base, for example by the procedure of Kowalski, C. J. and Fields, K. W. J. Org. Chem. (1981), 46, 197.

Compounds of formula (A) may be prepared from a compounds of formula (I) by hydrolysis, preferably in the presence of an acid catalyst such as hydrochloric acid and optionally in the presence of a suitable solvent such as tetrahydrofuran or acetone preferably between 25° C. and 150° C. under conventional heating or under microwave irradiation.

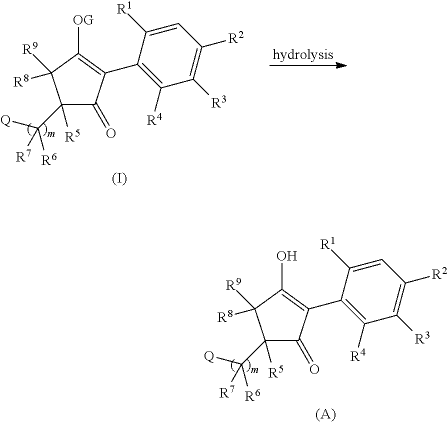

In a further approach, compounds of formula (A) may be prepared by the cyclisation of a compound of formula (B) or a compound of formula (C), wherein R′″ is hydrogen or an alkyl group, preferably in the presence of an acid or base, and optionally in the presence of a suitable solvent, by analogous methods to those described by T. N. Wheeler, U.S. Pat. No. 4,209,532. Compounds of formula (B) or compounds of formula (C) wherein R′″ is hydrogen may be cyclised under acidic conditions, preferably in the presence of a strong acid such as sulfuric acid, polyphosphoric acid or Eaton's reagent, optionally in the presence of a suitable solvent such as acetic acid, toluene or dichloromethane.

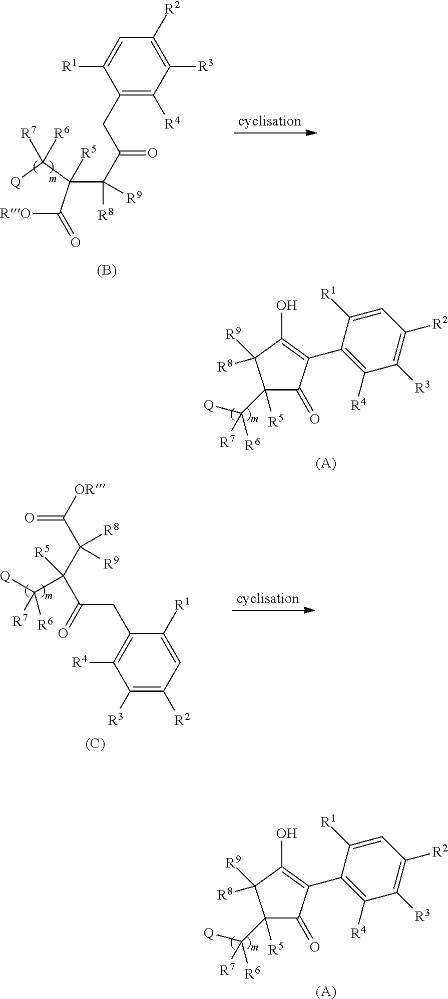

Compounds of formula (B) or compounds of formula (C) wherein R′″ is alkyl (preferably methyl or ethyl), may be cyclised under acidic or basic conditions, preferably in the presence of at least one equivalent of a strong base such as potassium tert-butoxide, lithium diisopropylamide or sodium hydride and in a solvent such as tetrahydrofuran, toluene, dimethylsulfoxide or N,N-dimethylformamide.

Compounds of formula (B) and compounds of formula (C), wherein R′″ is H, may be esterified to, respectively, compounds of formula (B) and compounds of formula (C), wherein R′″ is alkyl, under standard conditions, for example by heating with an alkyl alcohol, ROH, in the presence of an acid catalyst.

Compounds of formula (B) and compounds of formula (C), wherein R′″ is H, may be prepared, respectively, by saponification of a compounds of formula (D) and compounds of formula (E) wherein R′″″ is alkyl (preferably methyl or ethyl), under standard conditions, followed by acidification of the reaction mixture to effect decarboxylation, by similar processes to those described, for example, by T. N. Wheeler, U.S. Pat. No. 4,209,532.

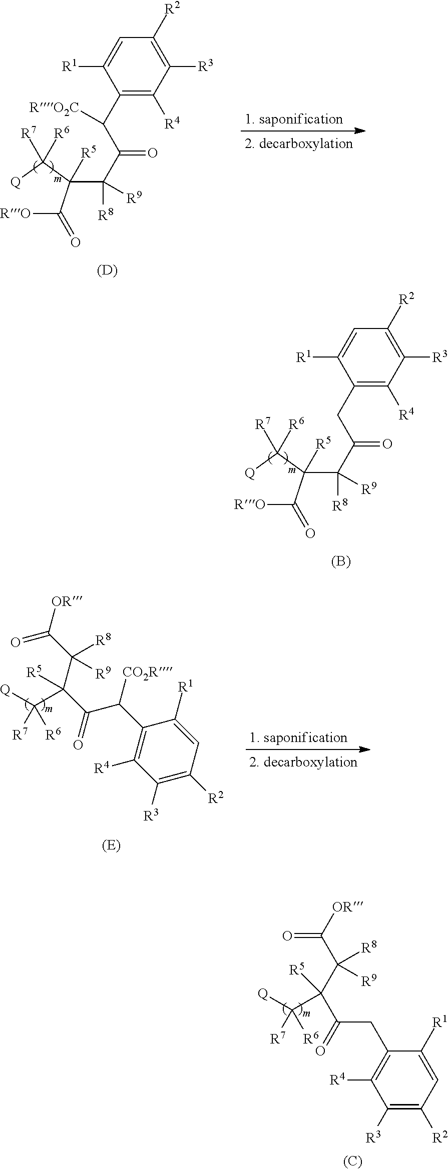

Compounds of formula (D) and compounds of formula (E), wherein R″″ is alkyl, may be prepared by treating, respectively, compounds of formula (F) with suitable carboxylic acid chlorides of formula (G) or suitable carboxylic acid chlorides of formula (H) under basic conditions. Suitable bases include potassium tert-butoxide, sodium bis(trimethylsilyl)amide and lithium diisopropylamide and the reaction is preferably conducted in a suitable solvent (such as tetrahydrofuran or toluene) at a temperature of between −80° C. and 30° C. Alternatively, compounds of formula (D) and compounds of formula (E), wherein R″″ is H, may be prepared by treating a compound of formula (F) with a suitable base (such as potassium tert-butoxide, sodium bis(trimethylsilyl)amide and lithium diisopropylamide) in a suitable solvent (such as tetrahydrofuran or toluene) at a suitable temperature (between −80° C. and 30° C.) and reacting the resulting anion with a suitable anhydride of formula (J):

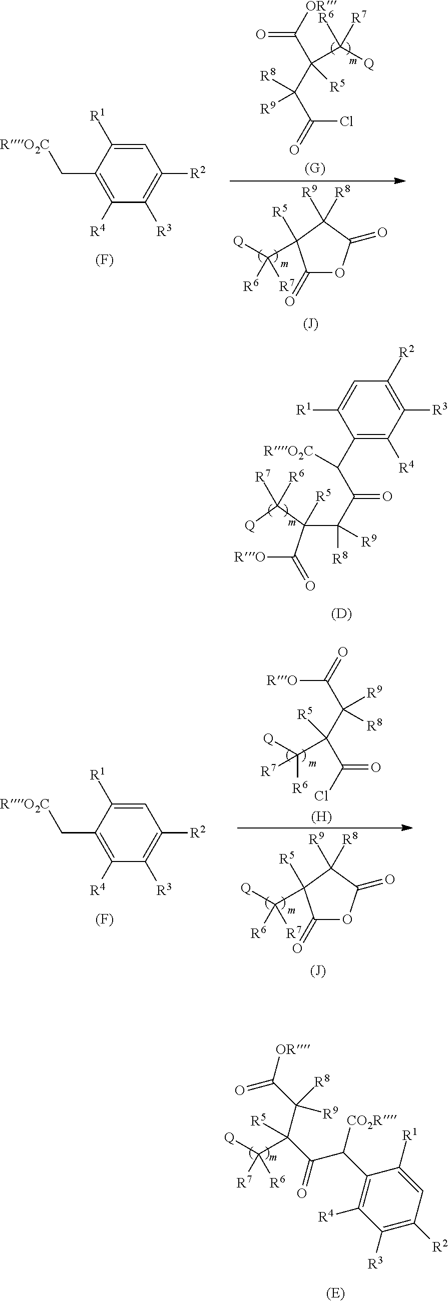

Compounds of formula (F) are known compounds, or may be prepared from known compounds by known methods.

Compounds of formula (J) may be prepared, for example, by analogous methods to those described by Ballini, R. et al. Synthesis (2002), (5), 681-685; Bergmeier, S. C. and Ismail, K. A. Synthesis (2000), (10), 1369-1371; Groutas, W. C. et al. J. Med. Chem. (1989), 32 (7), 1607-11 and Bernhard, K. and Lincke, H. Helv. Chim. Acta (1946), 29, 1457-1466.

Compounds of formula (G) or compounds of formula (H) may be prepared from a compound of formula (J) by treatment with an alkyl alcohol, R′″—OH, in the presence of a base, such as dimethylaminopyridine or an alkaline metal alkoxide (see, for example, Buser, S, and Vasella, A. Helv. Chim. Acta, (2005), 88, 3151 and M. Hart et al. Bioorg. Med. Chem. Letters, (2004), 14, 1969), followed by treatment of the resulting acid with a chlorinating reagent such as oxalyl chloride or thionyl chloride under known conditions (see, for example, Santelli-Rouvier. C. Tetrahedron Lett. (1984), 25 (39), 4371; Walba D. and Wand, M. Tetrahedron Lett. (1982), 23 (48), 4995; Cason, J. Org. Synth. Coll. Vol. III, (169), 1955).

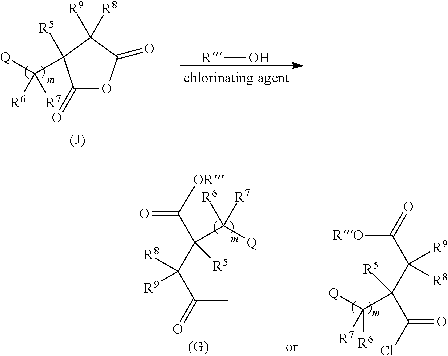

Compounds of formula (G) and compounds of formula (H) may be made from known compounds by known methods. For example, analogous methods to obtain compounds of formula (G) and compounds of formula (H) are described by Bergmeier, S. C. and Ismail, K. A. Synthesis (2000), (10), 1369-1371.

In an further approach to compounds of formula (I) may be prepared by treating compounds of formula (K) with compounds of formula (L) wherein LG is a leaving group such as halogen (preferably iodide or bromide) or an activated alcohol (preferably mesylate or tosylate) under basic conditions. Suitable bases include lithium diisopropylamide, sodium hexamethyldisilazide, potassium tert-butoxide and the reaction is preferably conducted in a suitable solvent (such as tetrahydrofuran) at a temperature between −80° C. and 30° C.

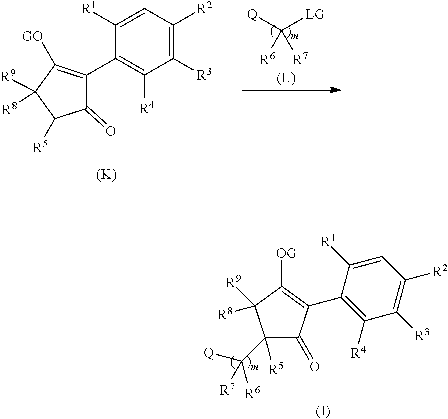

Compounds of formula (L) are known, or may be made known compounds by known methods (see for example: WO2006016178; Ueno, H. et al. J. Med. Chem. (2005), 48(10), 3586-3604; Kanoh, S. et al. Tetrahedron (2002), 58(35), 7049-7064; Strachan, J.-P. et al. J. Org. Chem. (2006), 71(26), 9909-9911).

Compounds of formula (K) are known compounds or may be made from known compounds by known methods (see, for example, Song, Y. S. S. et al. Tetrahedron Lett. (2005), 46 (46), 5987-5990; Kuethe, J. T. et al. J. Org. Chem. (2002), 67(17), 5993-6000).

Alternatively, compounds of formula (K) wherein G is C₁-C₆alkyl may be prepared by alkylation of compounds of formula (K), wherein G is hydrogen under known conditions or by known methods (see, for example, Eberhardt, U. et al. Chem. Ber. (1983), 116 (1), 119-135).

Compounds of formula (K), wherein G is hydrogen, are known, or may be prepared from known compounds by known methods (see, for example, Nguyen, H. N. et al. J. Am. Chem. Soc. (2003), 125 (39), 11818-11819; Bonjoch, J. et al. Tetrahedron (2001), 57(28), 6011-6017; Fox, J. M. et al. J. Am. Chem. Soc. (2000), 122(7), 1360-1370; U.S. Pat. No. 4,338,122; U.S. Pat. No. 4,283,348).

Alternatively, compounds of formula (I) where R⁵ and R⁶ form a bond can be prepared from compounds of formula (M) by known methods (see for example Nagaoka, H. et al. Tetrahedron Letters (1985), 26 (41), 5053-5056; Nagaoka, H. et al. J. Am. Chem. Soc. (1986), 108 (16), 5019-5021; Zuki, M. et al. Bull. Chem. Soc. Japan (1988), 61(4), 1299-1312; Enholm, E. J. et al. J. Org. Chem. (1996), 61 (16), 5384-5390; Clive, D. L. J. et al. Tetrahedron (2001), 57 (18), 3845-3858; Bartoli, G. et al. J. Org. Chem. (2002), 67 (25), 9111-9114. Jung, M. E. et al. Chem. Comm. (2003), (2), 196-197; EP1433772; JP2004203844; IN194295)

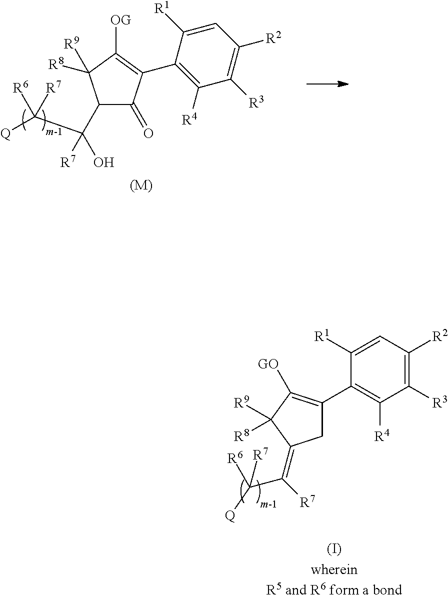

Compounds of formula (M) may be prepared by treating compounds of formula (K) (in which R⁵ is hydrogen) with compounds of formula (N) under basic conditions. Suitable bases include lithium diisopropylamide, sodium hexamethyldisilazide, potassium tert-butoxide and the reaction is preferably conducted in a suitable solvent (such as tetrahydrofuran) at a temperature between −80° C. and 30° C.

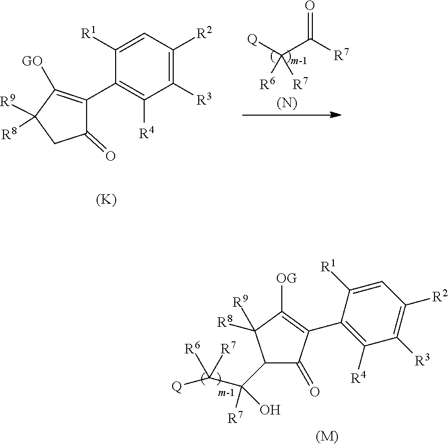

Compounds of formula (N) are known, or may be made from known compounds by known methods.

Compounds of formula (I) (wherein G is C₁-C₄alkyl) may be prepared by reacting a compounds of formula (O) (wherein G is C₁-C₄alkyl, and Hal is a halogen, preferably bromine or iodine), with aryl boronic acids, Ar—B(OH)₂ of formula (P) or aryl boronate esters in the presence of a suitable palladium catalyst (for example 0.001-50% palladium(II) acetate with respect to compound (O)) and a base (for example 1 to 10 equivalents potassium phosphate with respect to compound (O)) and preferably in the presence of a suitable ligand (for example 0.001-50% (2-dicyclohexylphosphino)-2′,6′-dimethoxybiphenyl with respect to compound (O)), and in a suitable solvent (for example toluene or 1,2-dimethoxyethane), preferably between 25° C. and 200° C. under conventional heating or under microwave irradiation (see, for example, Song, Y. S. S. et al. Tetrahedron Lett. (2005), 46 (46), 5987-5990; Kuethe, J. T. et al. J. Org. Chem. (2002), 67(17), 5993-6000).

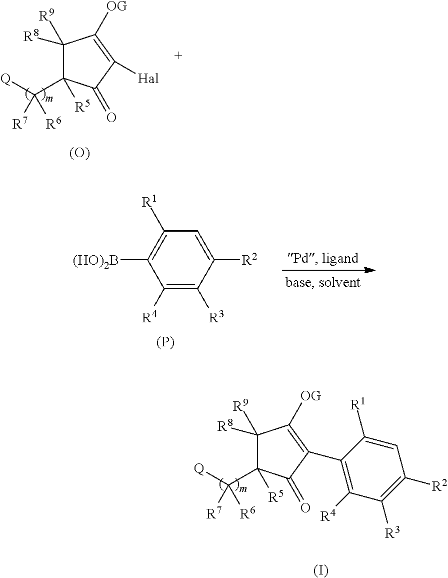

Compounds of formula (O) may be prepared by halogenating compounds of formula (Q), followed by alkylation of the resulting halide of formula (R) with a C₁-C₄alkyl halide or tri-C₁-C₄alkylorthoformate under known conditions, for example by the procedures of Shepherd R. G. et al. J. Chem. Soc. Perkin Trans. 1 (1987), 2153-2155 and Lin Y.-L. et al. Bioorg. Med. Chem. (2002), 10, 685-690. Alternatively, compounds of formula (O) may be prepared by alkylating a compound of formula (Q) with a C₁₋₄ alkyl halide or a tri-C₁₋₄-alkylorthoformate, and halogenating the resulting enone of formula (S) under known conditions (see for example Song, Y. S. et al. Tetrahedron Lett. (2005), 46 (36), 5987-5990; Kuethe, J. T. et al. J. Org. Chem. (2002), 67(17), 5993-6000; Belmont, D. T. et al. J. Org. Chem. 1985, 50 (21), 4102-4107).

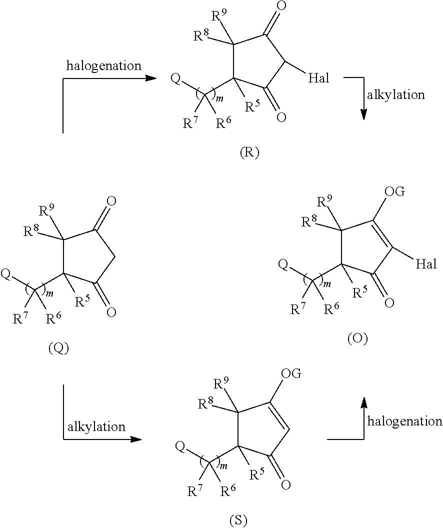

Compounds of formula (S) may be prepared by treating compounds of formula (T) with compounds of formula (L) wherein LG is a leaving group such as halogen (preferably iodide or bromide) or an activated alcohol (preferably mesylate or tosylate) under basic conditions. Suitable bases include lithium diisopropylamide, sodium hexamethyldisilazide, potassium tert-butoxide and the reaction is preferably conducted in a suitable solvent (such as tetrahydrofuran) at a temperature between −80° C. and 30° C. (see, for example, Gulias, M. et al. Org. Lett. (2003), 5(11), 1975-1977; Altenbach, R. J. et al. J. Med. Chem. (2006), 49 (23), 6869-6887; Snowden, R. L. Tetrahedron (1986), 42 (12), 3277-90; Oppolzer, W. et al. Helv. Chim. Acta (1980), 63 (4), 788-92; Mellor, M. et al. Synth. Commun. 1979, 9 (1), 1-4).

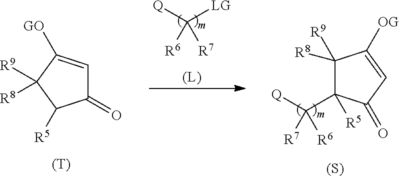

Compounds of formula (T) are known, or may be made from known compounds by known methods.

Alternatively compounds of formula (S) where R⁵ and R⁶ from a bond can be prepared from compounds of formula (U) by known methods (see, for example, Nagaoka, H. et al. Tetrahedron Letters (1985), 26 (41), 5053-5056; Nagaoka, H. et al. J. Am. Chem. Soc. (1986), 108 (16), 5019-5021; Zuki, M. et al. Bull. Chem. Soc. Japan (1988), 61(4), 1299-1312; Enholm, E. J. et al. J. Org. Chem. (1996), 61 (16), 5384-5390; Clive, D. L. J. et al. Tetrahedron (2001), 57 (18), 3845-3858; Bartoli, G. et al. J. Org. Chem. (2002), 67 (25), 9111-9114. Jung, M. E. et al. Chem. Comm. (2003), (2), 196-197; EP1433772; JP2004203844; IN194295).

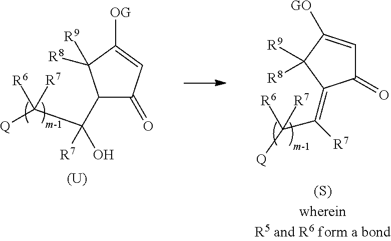

Compounds of formula (U) may be prepared by treating compounds of formula (T) with compounds of formula (N) under basic conditions. Suitable bases include lithium diisopropylamide, sodium hexamethyldisilazide, potassium tert-butoxide and the reaction is preferably conducted in a suitable (such as tetrahydrofuran) at a temperature between −80° C. and 30° C. (see, for example, Aleman, J. et al. Chem. Comm. (2007), (38), 3921-3923).

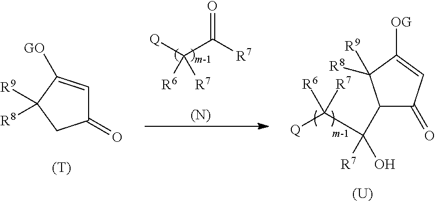

Compounds of formula (P) may be prepared from an aryl halide of formula (V), wherein Hal is bromine or iodine, by known methods (see, for example, Thompson W. et al. J. Org. Chem. (1984), 49, 5237 and R. Hawkins et al. J. Am. Chem. Soc. (1960), 82, 3053). For example, an aryl halide of formula (V) may be treated with an alkyl lithium or alkyl magnesium halide in a suitable solvent, preferably diethyl ether or tetrahydrofuran, at a temperature of between −80° C. and 30° C., and the aryl magnesium or aryl lithium reagent obtained may then be reacted with a trialkyl borate (preferably trimethylborate) to give an aryl dialkylboronate which may be hydrolysed to provide a boronic acid of formula (P) under acidic conditions.

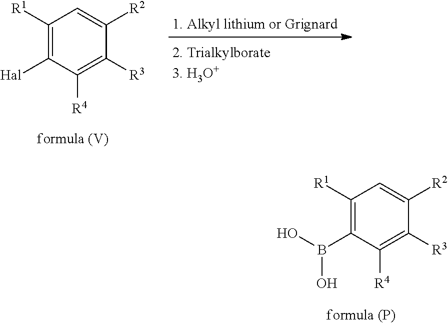

Alternatively a compound of formula (V) may be reacted with a cyclic boronate ester derived from a 1,2- or a 1,3-alkanediol such as pinacol, 2,2-dimethyl-1,3-propanediol and 2-methyl-2,4-pentanediol) under known conditions (see, for example, Miyaura N. et al. J. Org. Chem. (1995), 60, 7508, and Zhu W. et al. Org. Lett. (2006), 8 (2), 261), and the resulting boronate ester may be hydrolysed under acidic conditions to give a boronic acid of formula (P).

Aryl halides of formula (V) are known, or may be prepared from known compounds by known methods. For example, aryl halides of formula (V) may be prepared from anilines of formula (W) by known methods, for example the Sandmeyer reaction, via the corresponding diazonium salts.

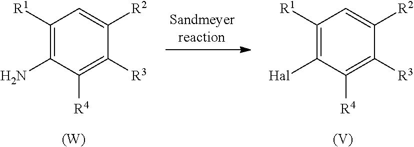

Anilines of formula (W) are known compounds, or may be made from known compounds, by known methods.

Alternatively compounds of formula (V) can be made by halogenations of the corresponding known compounds, by known methods.

Compounds of formula (Q) may be prepared from compounds of formula (S) by hydrolysis, preferably in the presence of an acid catalyst such as hydrochloric acid and optionally in the presence of a suitable solvent such as tetrahydrofuran or acetone preferably between 25° C. and 150° C. under conventional heating or under microwave irradiation.

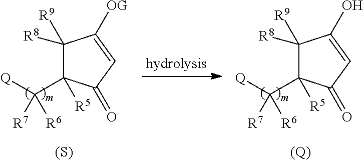

Alternatively, compounds of formula (Q) can be made from known compounds by known methods (see for example Manukina, T. A. et al. Zhurnal Organicheskoi Khimii (1986), 22(4), 873-4; Mellor, M. et al. Synth. Commun. 1979, 9 (1), 1-4).

In a further approach, compounds of formula (A) may be prepared by reacting compounds of formula (Q) with suitable aryl halides (such as aryl-iodides, aryl-bromides or aryl-chlorides), Ar—Hal of formula (V), or suitable C₁-C₆alkylsulfonates (preferably mesylate) or C₁-C₆haloalkylsulfonates (preferably triflate) or an arylsulfonates (preferable tosylate) in the presence of a suitable palladium catalyst (for example 0.001-50% palladium(II) acetate with respect to compounds of formula (Q)) and a base (for example 1 to 10 equivalents potassium phosphate with respect to compounds of formula (Q)) and preferably in the presence of a suitable ligand (for example 0.001-50% (2-dicyclohexylphosphino)-2′,4′,6′-triisopropylbiphenyl with respect to compounds of formula (Q)), and in a suitable solvent (for example dioxane or 1,2-dimethoxyethane), preferably between 25° C. and 200° C. Similar couplings are known in the literature (see for example, Belmont, D. T. et al. J. Org. Chem. 1985, 50 (21), 4102-4107; Fox, J. M. et al. J. Am. Chem. Soc. (2000), 122 (7), 1360-1370; B. Hong et al. WO 2005/000233). Alternatively, compounds of formula (A) may be prepared by reacting compounds of formula (Q) with suitable aryl halides (such as an aryl-iodides), Ar-Hal of formula (V), in the presence of a suitable copper catalyst (for example 0.001-50% copper(I) iodide with respect to compounds of formula (Q)) and a base (for example 1 to 10 equivalents potassium carbonate with respect to compounds of formula (Q)) and preferably in the presence of a suitable ligand (for example 0.001-50% L-proline with respect to compounds of formula (Q)), and in a suitable solvent (for example dimethylsulfoxide), preferably between 25° C. and 200° C. Similar couplings are known in the literature for aryl halides (see, for example, Jiang, Y. et al. Synlett (2005), 18, 2731-2734).

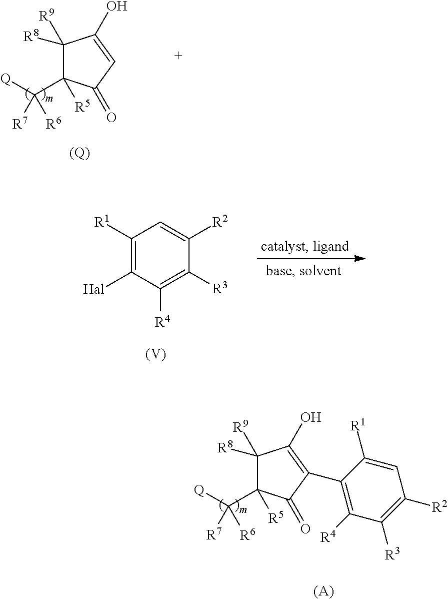

Additional compounds of formula (A) may be prepared by reacting compounds of formula (Q) with organolead reagents of formula (X) under conditions described, for example, by Pinhey, J. Pure and Appl. Chem. (1996), 68 (4), 819 and by Moloney M. et al. Tetrahedron Lett. (2002), 43, 3407.

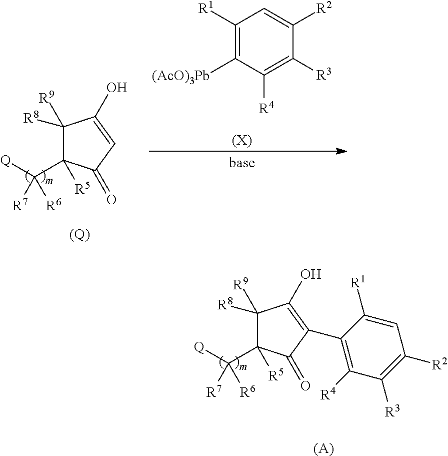

The organolead reagent of formula (X) may be prepared from a boronic acid of formula (P), a stannane of formula (Y), wherein R′″″ is C₁-C₄ alkyl or by direct plumbation of a compound of formula (Z) with lead tetraacetate according to known procedures.

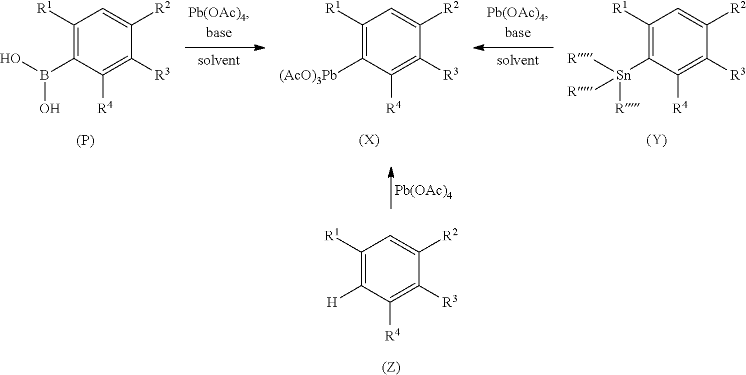

Further compounds of formula (A) may be prepared by reacting compounds of formula (Q) with suitable triarylbismuth compounds under conditions described, for example, by Fedorov, A. U. et al. Russ. Chem. Bull. Int. Ed. (2005), 54 (11), 2602 and by Koech P. et al. J. Am. Chem. Soc. (2004), 126 (17), 5350 and references therein.

Additional compounds of formula (A) may be prepared by reacting an iodonium ylide of formula (AA), wherein Ar is an optionally substituted phenyl group, and an aryl boronic acid of formula (P), in the presence of a suitable palladium catalyst, a base and in a suitable solvent.

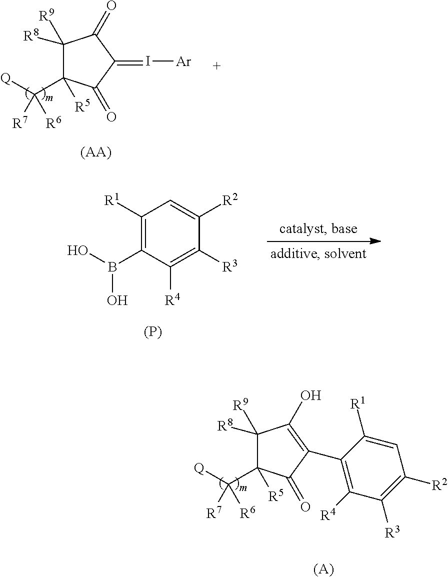

Suitable palladium catalysts are generally palladium(II) or palladium(0) complexes, for example palladium(II) dihalides, palladium(II) acetate, palladium(II) sulfate, bis(triphenylphosphine)-palladium(II) dichloride, bis(tricyclopentylphosphine)palladium(II) dichloride, bis(tricyclohexyl-phosphine)palladium(II) dichloride, bis(dibenzylideneacetone)palladium(0) or tetrakis-(triphenylphosphine)palladium(0). The palladium catalyst can also be prepared in situ from palladium(II) or palladium(0) compounds by complexing with the desired ligands, by, for example, combining the palladium(II) salt to be complexed, for example palladium(II) dichloride (PdCl₂) or palladium(II) acetate (Pd(OAc)₂), together with the desired ligand, for example triphenylphosphine (PPh₃), tricyclopentylphosphine, tricyclohexylphosphine, 2-dicyclohexylphosphino-2′,6′-dimethoxybiphenyl or 2-dicyclohexylphosphino-2′,4′,6′-triisopropylbiphenyl and the selected solvent, with a compound of formula (AA), the arylboronic acid of formula (P), and a base. Also suitable are bidendate ligands, for example 1, 1′-bis(diphenylphosphino)ferrocene or 1,2-bis(diphenylphosphino)ethane. By heating the reaction medium, the palladium(II) complex or palladium(0) complex desired for the C—C coupling reaction is thus formed in situ, and then initiates the C—C coupling reaction.

The palladium catalysts are used in an amount of from 0.001 to 50 mol %, preferably in an amount of from 0.1 to 15 mol %, based on the compound of formula (AA). The reaction may also be carried out in the presence of other additives, such as tetralkylammonium salts, for example, tetrabutylammonium bromide. Preferably the palladium catalyst is palladium acetate, the base is lithium hydroxide and the solvent is aqueous 1,2-dimethoxyethane.

A compound of formula (AA) may be prepared from a compound of formula (Q) by treatment with a hypervalent iodine reagent such as a (diacetoxy)iodobenzene or an iodosylbenzene and a base such as aqueous sodium carbonate, lithium hydroxide or sodium hydroxide in a solvent such as water or an aqueous alcohol such as aqueous ethanol according to the procedures of Schank K. et al. Synthesis (1983), 392, Moriarty R. M. et al. J. Am. Chem. Soc. (1985), 107, 1375 or of Yang Z. et al. Org. Lett. (2002), 4 (19), 3333.

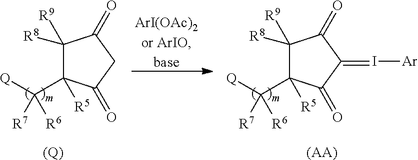

Additional compounds of formula (A) may be prepared by the pinacol rearrangement of compounds of formula (AB) or compounds of formula (AC) wherein R′″″″ is C₁-C₄ alkyl (preferably methyl) under acidic conditions (see, for example, Eberhardt, U. et. al. Chem. Ber. (1983), 116(1), 119-35 and Wheeler, T. N. U.S. Pat. No. 4,283,348)

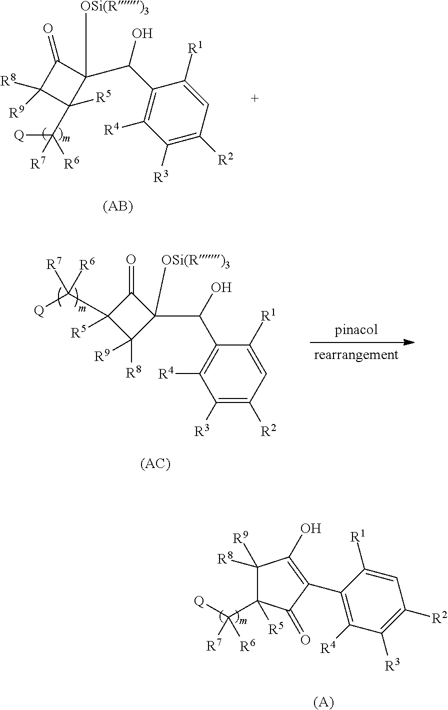

Compounds of formula (AB) and compounds of formula (AC) may be prepared by treating compounds of formula (AD) with compounds of formula (AE) in the presence of an acid (such as titanium tetrachloride or magnesium iodide) optionally in a suitable solvent (such as dichloromethane) at a temperature between −80° C. and 30° C. (see, for example, Li, W.-D. Z. and Zhang, X.-X. Org. Lett. (2002), 4(20), 3485-3488; Shimada, J. et al. J. Am. Chem. Soc. (1984), 106(6), 1759-73; Eberhardt, U. et. al. Chem. Ber. (1983), 116(1), 119-35 and Wheeler, T. N. U.S. Pat. No. 4,283,348).

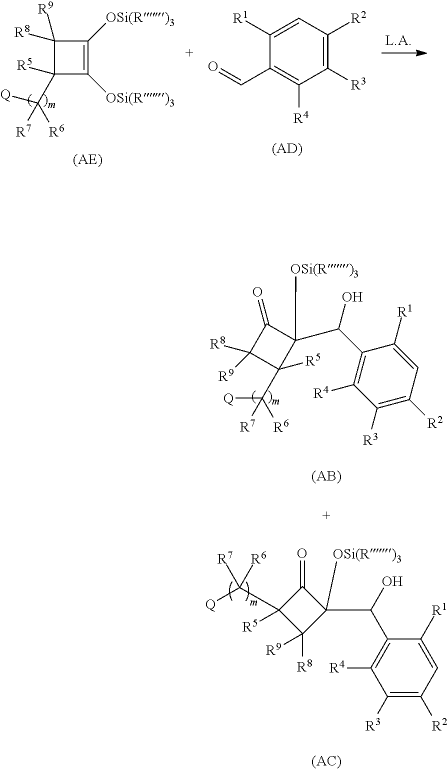

Compounds of formula (AD) are known or may be made by known methods from compounds of formula (V) or compounds of formula (Z).

Compounds of formula (AE) may be prepared from compounds of formula (AF) where in R′″ is an alkyl group (preferably methyl) in the presence of chloro tri-C₁-C₄alkyl silyl and a metal (preferably sodium) in a suitable solvent (such as toluene or diethyl ether) at a temperature between 20° C. and 150° C. (see, for example, Blanchard, A. N. and Burnell, D. J. Tetrahedron Lett. (2001), 42(29), 4779-4781 and Salaun, J. et al. Tetrahedron (1989), 45(10), 3151-62).

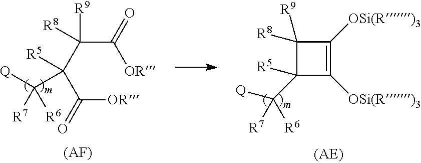

Compounds of formula (AF) are analogous to compounds of formula (H) and compounds of formula (G) and may be prepared by know methods analogous to those describe for compounds of formula (H) and compounds of formula (G).

Additional compounds of formula (I) may be prepared wherein R⁵ and R⁶ form a bond and R⁷ R⁷ is C₁-C₆alkylsulfonate (preferably mesylate) or C₁-C₆haloalkylsulfonate (preferably triflate) or an arylsulfonate (preferable tosylate) may be prepared from compounds of formula (AG) following known procedures (Specklin et al. J. Org. Chem. 2008, 73(19), 7845-7848).

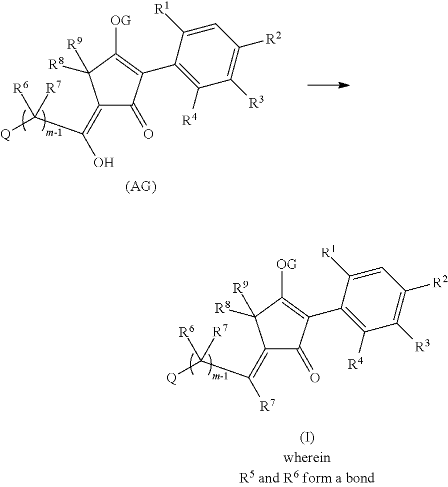

Compounds of formula (AG) may be prepared from compounds of formula (AH) under basic or acidic conditions. For example of a procedure see G. Quinkert et al. Helv. Chim. Acta, 1986, 69(3), 469-537.

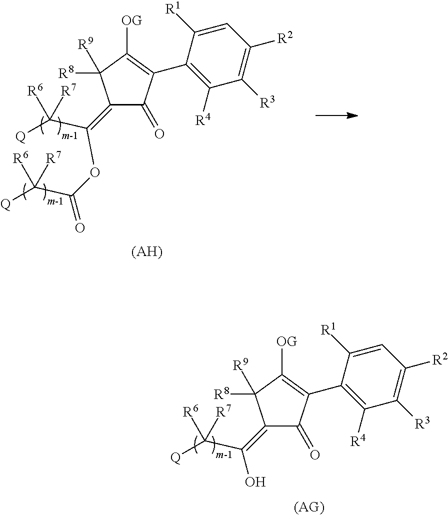

Compounds of formula (AH) may be prepared by reaction of compounds of formula (K) wherein R⁵ is hydrogen with acids chloride of formula (AJ) in the presence of a base.

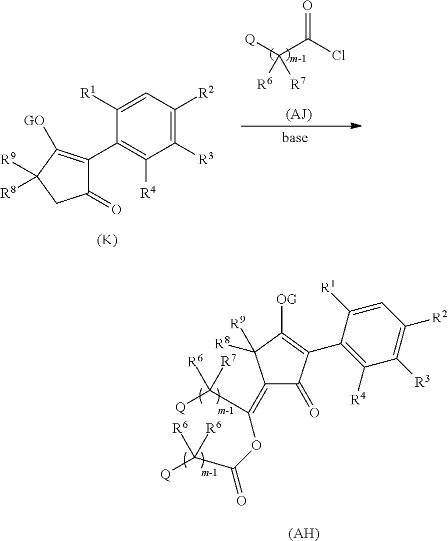

Compounds of formula (AJ) are known or may be made by known methods from known compounds.

Alternatively, compounds of formula (AG) can be prepared from compounds of formula (M) using known oxidative procedures (see for example D. B. Dess and J. C. Martin J. Org. Chem. 1983, 48 (22), 4155-4156).

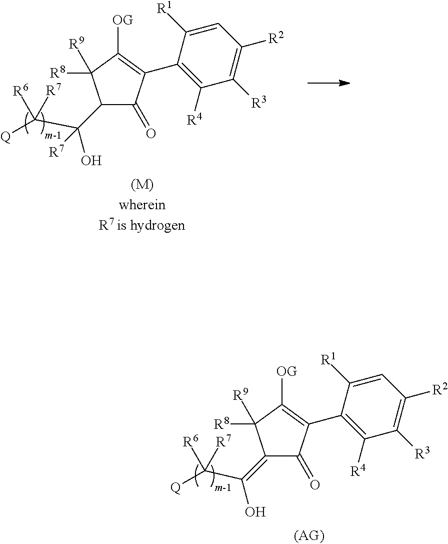

A compound I can be converted in a manner known per se into another compound I by replacing one or more substituents of the starting compound I in the customary manner by (an)other substituent(s) according to the invention.

Depending on the choice of the reaction conditions and starting materials which are suitable in each case, it is possible, for example, in one reaction step only to replace one substituent by another substituent according to the invention, or a plurality of substituents can be re-placed by other substituents according to the invention in the same reaction step.

Salts of compounds I can be prepared in a manner known per se. Thus, for example, acid addition salts of compounds I are obtained by treatment with a suitable acid or a suitable ion exchanger reagent and salts with bases are obtained by treatment with a suitable base or with a suitable ion exchanger reagent.

Salts of compounds I can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.

Salts of compounds I can be converted in a manner known per se into other salts of compounds I, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds I, which have salt-forming properties can be obtained in free form or in the form of salts.

The compounds I and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds I, in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds I and, where appropriate, the tautomers thereof, in each case in free form or in salt form, can, if appropriate, also be obtained in the form of hydrates and/or include other solvents, for example those which may have been used for the crystallization of compounds which are present in solid form.

The compounds according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i.e. in destruction of the pests, which takes place either immediately or only after some time has elapsed, for example during ecdysis, or indirectly, for example in a reduced oviposition and/or hatching rate, a good activity corresponding to a destruction rate (mortality) of at least 50 to 60%.

The compounds of formula I can be used to combat and control infestations of insect pests such as Lepidoptera, Diptera, Hemiptera, Thysanoptera, Orthoptera, Dictyoptera, Coleoptera, Siphonaptera, Hymenoptera and Isoptera and also other invertebrate pests, for example, acarine, nematode and mollusc pests. Insects, acarines, nematodes and molluscs are hereinafter collectively referred to as pests. The pests which may be combated and controlled by the use of the invention compounds include those pests associated with agriculture (which term includes the growing of crops for food and fibre products), horticulture and animal husbandry, companion animals, forestry and the storage of products of vegetable origin (such as fruit, grain and timber); those pests associated with the damage of man-made structures and the transmission of diseases of man and animals; and also nuisance pests (such as flies).

Examples of pest species which may be controlled by the compounds of formula I include: Myzus persicae (aphid), Aphis gossypii (aphid), Aphis fabae (aphid), Lygus spp. (capsids), Dysdercus spp. (capsids), Nilaparvata lugens (planthopper), Nephotettixc incticeps (leafhopper), Nezara spp. (stinkbugs), Euschistus spp. (stinkbugs), Leptocorisa spp. (stinkbugs), Frankliniella occidentalis (thrip), Thrips spp. (thrips), Leptinotarsa decemlineata (Colorado potato beetle), Anthonomus grandis (boll weevil), Aonidiella spp. (scale insects), Trialeurodes spp. (white flies), Bemisia tabaci (white fly), Ostrinia nubilalis (European corn borer), Spodoptera littoralis (cotton leafworm), Heliothis virescens (tobacco budworm), Helicoverpa armigera (cotton bollworm), Helicoverpa zea (cotton bollworm), Sylepta derogata (cotton leaf roller), Pieris brassicae (white butterfly), Plutella xylostella (diamond back moth), Agrotis spp. (cutworms), Chilo suppressalis (rice stem borer), Locusta migratoria (locust), Chortiocetes terminifera (locust), Diabrotica spp. (rootworms), Panonychus ulmi (European red mite), Panonychus citri (citrus red mite), Tetranychus urticae (two-spotted spider mite), Tetranychus cinnabarinus (carmine spider mite), Phyllocoptruta oleivora (citrus rust mite), Polyphagotarsonemus latus (broad mite), Brevipalpus spp. (flat mites), Boophilus microplus (cattle tick), Dermacentor variabilis (American dog tick), Ctenocephalides felis (cat flea), Liriomyza spp. (leafminer), Musca domestica (housefly), Aedes aegypti (mosquito), Anopheles spp. (mosquitoes), Culex spp. (mosquitoes), Lucillia spp. (blowflies), Blattella germanica (cockroach), Periplaneta americana (cockroach), Blatta orientalis (cockroach), termites of the Mastotermitidae (for example Mastotermes spp.), the Kalotermitidae (for example Neotermes spp.), the Rhinotermitidae (for example Coptotermes formosanus, Reticulitermes flavipes, R. speratu, R. virginicus, R. hesperus, and R. santonensis) and the Termitidae (for example Globitermes sulphureus), Solenopsis geminata (fire ant), Monomorium pharaonis (pharaoh's ant), Damalinia spp. and Linognathus spp. (biting and sucking lice), Meloidogyne spp. (root knot nematodes), Globodera spp. and Heterodera spp. (cyst nematodes), Pratylenchus spp. (lesion nematodes), Rhodopholus spp. (banana burrowing nematodes), Tylenchulus spp. (citrus nematodes), Haemonchus contortus (barber pole worm), Caenorhabditis elegans (vinegar eelworm), Trichostrongylus spp. (gastro intestinal nematodes) and Deroceras reticulatum (slug).

Further examples of the above mentioned pests are:

from the order Acarina, for example,
Acarus siro, Aceria sheldoni, Aculus schlechtendali, Amblyomma spp., Argas spp., Boophilus spp., Brevipalpus spp., Bryobia praetiosa, Calipitrimerus spp., Chorioptes spp., Dermanyssus gallinae, Eotetranychus carpini, Eriophyes spp., Hyalomma spp., Ixodes spp., Olygonychus pratensis, Ornithodoros spp., Panonychus spp., Phyllocoptruta oleivora, Polyphagotarsonemus latus, Psoroptes spp., Rhipicephalus spp., Rhizoglyphus spp., Sarcoptes spp., Tarsonemus spp. and Tetranychus spp.;
from the order Anoplura, for example,

### Haematopinus spp., Linognathus spp., Pediculus spp., Pemphigus spp. and Phylloxera spp.;

from the order Coleoptera, for example,
Agriotes spp., Anthonomus spp., Atomaria linearis, Chaetocnema tibialis, Cosmopolites spp., Curculio spp., Dermestes spp., Diabrotica spp., Epilachna spp., Eremnus spp., Leptinotarsa decemLineata, Lissorhoptrus spp., Melolontha spp., Orycaephilus spp., Otiorhynchus spp., Phlyctinus spp., Popillia spp., Psylliodes spp., Rhizopertha spp., Scarabeidae, Sitophilus spp., Sitotroga spp., Tenebrio spp., Tribolium spp. and Trogoderma spp.;
from the order Diptera, for example,
Aedes spp., Antherigona soccata, Bibio hortulanus, Calliphora erythrocephala, Ceratitis spp., Chrysomyia spp., Culex spp., Cuterebra spp., Dacus spp., Drosophila melanogaster, Fannia spp., Gastrophilus spp., Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis pomonella, Sciara spp., Stomoxys spp., Tabanus spp., Tannia spp. and Tipula spp.;
from the order Heteroptera, for example,
Cimex spp., Distantiella theobroma, Dysdercus spp., Euchistus spp., Eurygaster spp., Leptocorisa spp., Nezara spp., Piesma spp., Rhodnius spp., Sahlbergella singularis, Scotinophara spp. and Triatoma spp.;
from the order Homoptera, for example,
Aleurothrixus floccosus, Aleyrodes brassicae, Aonidiella spp., Aphididae, Aphis spp., Aspidiotus spp., Bemisia tabaci, Ceroplaster spp., Chrysomphalus aonidium, Chrysomphalus dictyospermi, Coccus hesperidum, Empoasca spp., Eriosoma larigerum, Erythroneura spp., Gascardia spp., Laodelphax spp., Lecanium corni, Lepidosaphes spp., Macrosiphus spp., Myzus spp., Nephotettix spp., Nilaparvata spp., Parlatoria spp., Pemphigus spp., Planococcus spp., Pseudaulacaspis spp., Pseudococcus spp., Psylla spp., Pulvinaria aethiopica, Quadraspidiotus spp., Rhopalosiphum spp., Saissetia spp., Scaphoideus spp., Schizaphis spp., Sitobion spp., Trialeurodes vaporariorum, Trioza erytreae and Unaspis citri;
from the order Hymenoptera, for example,
Acromyrmex, Atta spp., Cephus spp., Diprion spp., Diprionidae, Gilpinia polytoma, Hoplocampa spp., Lasius spp., Monomorium pharaonis, Neodiprion spp., Solenopsis spp. and Vespa spp.;
from the order Isoptera, for example,

### Reticulitermes spp.;

from the order Lepidoptera, for example,
Acleris spp., Adoxophyes spp., Aegeria spp., Agrotis spp., Alabama argillaceae, Amylois spp., Anticarsia gemmatalis, Archips spp., Argyrotaenia spp., Autographa spp., Busseola fusca, Cadra cautella, Carposina nipponensis, Chilo spp., Choristoneura spp., Clysia ambiguella, Cnaphalocrocis spp., Cnephasia spp., Cochylis spp., Coleophora spp., Crocidolomia binotalis, Cryptophlebia leucotreta, Cydia spp., Diatraea spp., Diparopsis castanea, Earias spp., Ephestia spp., Eucosma spp., Eupoecilia ambiguella, Euproctis spp., Euxoa spp., Grapholita spp., Hedya nubiferana, Heliothis spp., Hellula undalis, Hyphantria cunea, Keiferia lycopersicella, Leucoptera scitella, Lithocollethis spp., Lobesia botrana, Lymantria spp., Lyonetia spp., Malacosoma spp., Mamestra brassicae, Manduca sexta, Operophtera spp., Ostrinia nubilalis, Pammene spp., Pandemis spp., Panolis flammea, Pectinophora gossypiela, Phthorimaea operculella, Pieris rapae, Pieris spp., Plutella xylostella, Prays spp., Scirpophaga spp., Sesamia spp., Sparganothis spp., Spodoptera spp., Synanthedon spp., Thaumetopoea spp., Tortrix spp., Trichoplusia ni and Yponomeuta spp.;
from the order Mallophaga, for example,

### Damalinea spp. and Trichodectes spp.;

from the order Orthoptera, for example,
Blatta spp., Blattella spp., Gryllotalpa spp., Leucophaea maderae, Locusta spp., Periplaneta spp. and Schistocerca spp.;
from the order Psocoptera, for example,

### Liposcelis spp.;

from the order Siphonaptera, for example,
Ceratophyllus spp., Ctenocephalides spp. and Xenopsylla cheopis;
from the order Thysanoptera, for example,
Frankliniella spp., Hercinothrips spp., Scirtothrips aurantii, Taeniothrips spp., Thrips palmi and Thrips tabaci; and
from the order Thysanura, for example,
Lepisma saccharina.

The active ingredients according to the invention can be used for controlling, i.e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family, latex plants and ornamentals.

The term “crops” is to be understood as including also crops that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady® and LibertyLink®.

The term “crops” is also to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popliae; or insecticidal proteins from Bacillus thuringiensis, such as δ-endotoxins, e.g. CryIA(b), CryIA(c), CryIF, CryIF(a2), CryIIA(b), CryIIIA, CryIIIB(b1) or Cry9c, or vegetative insecticidal proteins (VIP), e.g. VIP1, VIP2, VIP3 or VIP3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsine inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by δ-endotoxins, for example CryIA(b), CryIA(c), CryIF, CryIF(a2), CryIIA(b), CryIIIA, CryIIIB(b1) or Cry9c, or vegetative insecticidal proteins (VIP), for example VIP1, VIP2, VIP3 or VIP3A, expressly also hybrid toxins, truncated toxins and modified toxins. Hybrid toxins are produced recombinantly by a new combination of different domains of those proteins (see, for example, WO 02/15701). Truncated toxins, for example a truncated CryIA(b), are known. In the case of modified toxins, one or more amino acids of the naturally occurring toxin are replaced. In such amino acid replacements, preferably non-naturally present protease recognition sequences are inserted into the toxin, such as, for example, in the case of CryIIIA055, a cathepsin-D-recognition sequence is inserted into a CryIIIA toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. CryI-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available.

Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); Nature-Gard®Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated CryIA(b) toxin. Bt11 maize also transgenically expresses the enzyme PAT to achieve tolerance to the herbicide glufosinate ammonium.
2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a CryIA(b) toxin. Bt176 maize also transgenically expresses the enzyme PAT to achieve tolerance to the herbicide glufosinate ammonium.
3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified CryIIIA toxin. This toxin is Cry3A055 modified by insertion of a cathepsin-D-protease recognition sequence. The preparation of such transgenic maize plants is described in WO 03/018810.
4. MON 863 Maize from Monsanto Europe S.A. 270-272 Avenue de Tervuren, B-1150. Brussels, Belgium, registration number C/DE/02/9. MON 863 expresses a CryIIIB(b1) toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a CryIA(b) toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit and Nachhaltigkeit, Zentrum BATS, Clarastrasse 13, 4058 Basel, Switzerland) Report 2003.

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compounds and compositions according to the invention are the protection of stored goods and storerooms and the protection of raw materials, such as wood, textiles, floor coverings or buildings, and also in the hygiene sector, especially the protection of humans, domestic animals and productive livestock against pests of the mentioned type.

In the hygiene sector, the compounds and compositions according to the invention are active against ectoparasites such as hard ticks, soft ticks, mange mites, harvest mites, flies (biting and licking), parasitic fly larvae, lice, hair lice, bird lice and fleas.

Examples of such parasites are:

Of the order Anoplurida: Haematopinus spp., Linognathus spp., Pediculus spp. and Phtirus spp., Solenopotes spp.
Of the order Mallophagida: Trimenopon spp., Menopon spp., Trinoton spp., Bovicola spp., Werneckiella spp., Lepikentron spp., Damalina spp., Trichodectes spp. and Felicola spp.
Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.
Of the order Siphonapterida, for example Pulex spp., Ctenocephalides spp., Xenopsylla spp., Ceratophyllus spp.
Of the order Heteropterida, for example Cimex spp., Triatoma spp., Rhodnius spp., Panstrongylus spp.
Of the order Blattarida, for example Blatta orientalis, Periplaneta americana, Blattelagermanica and Supella spp.
Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.
Of the orders Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compounds and compositions according to the invention are also suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The invention therefore provides a method of combating and controlling insects, acarines, nematodes or molluscs which comprises applying an insecticidally, acaricidally, nematicidally or molluscicidally effective amount of a compound of formula I, or a composition containing a compound of formula I, to a pest, a locus of pest, or to a plant susceptible to attack by a pest, The compounds of formula I are preferably used against insects or acarines.

The term “plant” as used herein includes seedlings, bushes and trees.

Besides displaying good insecticidal and acaricidal action and properties, the active ingredient according to the invention are characterized by good plant/crop compatibility. Under different methods of application, the compounds of the formula I, or compositions thereof according to the invention, demonstrate good plant/crop tolerance whereby plant/crop damage (phytotoxicity) is significantly reduced. The terms “crop” and “plant” are to be understood as defined above, whereas the term “methods of application” is referred to below.

The invention therefore also relates to pesticidal compositions such as emulsifiable concentrates, suspension concentrates, directly sprayable or dilutable solutions, spreadable pastes, dilute emulsions, soluble powders, dispersible powders, wettable powders, dusts, granules or encapsulations in polymeric substances, which comprise—at least—one of the active ingredients according to the invention and which are to be selected to suit the intended aims and the prevailing circumstances.

In these compositions, the active ingredient is employed in pure form, a solid active ingredient for example in a specific particle size, or, preferably, together with—at least—one of the auxiliaries conventionally used in the art of formulation, such as extenders, for example solvents or solid carriers, or such as surface-active compounds (surfactants).

Examples of suitable solvents are: unhydrogenated or partially hydrogenated aromatic hydrocarbons, preferably the fractions C8 to C12 of alkylbenzenes, such as xylene mixtures, alkylated naphthalenes or tetrahydronaphthalene, aliphatic or cycloaliphatic hydrocarbons, such as paraffins or cyclohexane, alcohols such as ethanol, propanol or butanol, glycols and their ethers and esters such as propylene glycol, dipropylene glycol ether, ethylene glycol or ethylene glycol monomethyl ether or ethylene glycol monoethyl ether, ketones, such as cyclohexanone, isophorone or diacetone alcohol, strongly polar solvents, such as N-methylpyrrolid-2-one, dimethyl sulfoxide or N,N-dimethylformamide, water, unepoxidized or epoxidized vegetable oils, such as unexpodized or epoxidized rapeseed, castor, coconut or soya oil, and silicone oils.

Solid carriers which are used for example for dusts and dispersible powders are, as a rule, ground natural minerals such as calcite, talc, kaolin, montmorillonite or attapulgite. To improve the physical properties, it is also possible to add highly disperse silicas or highly disperse absorbtive polymers. Suitable particulate adsorptive carriers for granules are porous types, such as pumice, brick grit, sepiolite or bentonite, and suitable non-sorptive carrier materials are calcite or sand. In addition, a large number of granulated materials of inorganic or organic nature can be used, in particular dolomite or comminuted plant residues.

Suitable surface-active compounds are, depending on the type of the active ingredient to be formulated, non-ionic, cationic and/or anionic surfactants or surfactant mixtures which have good emulsifying, dispersing and wetting properties. The surfactants mentioned below are only to be considered as examples; a large number of further surfactants which are conventionally used in the art of formulation and suitable according to the invention are described in the relevant literature.

Suitable non-ionic surfactants are, especially, polyglycol ether derivatives of aliphatic or cycloaliphatic alcohols, of saturated or unsaturated fatty acids or of alkyl phenols which may contain approximately 3 to approximately 30 glycol ether groups and approximately 8 to approximately 20 carbon atoms in the (cyclo)aliphatic hydrocarbon radical or approximately 6 to approximately 18 carbon atoms in the alkyl moiety of the alkyl phenols. Also suitable are water-soluble polyethylene oxide adducts with polypropylene glycol, ethylenediaminopo-lypropylene glycol or alkyl polypropylene glycol having 1 to approximately 10 carbon atoms in the alkyl chain and approximately 20 to approximately 250 ethylene glycol ether groups and approximately 10 to approximately 100 propylene glycol ether groups. Normally, the abovementioned compounds contain 1 to approximately 5 ethylene glycol units per propy-lene glycol unit. Examples which may be mentioned are nonylphenoxypolyethoxyethanol, castor oil polyglycol ether, polypropylene glycol/polyethylene oxide adducts, tributylpheno-xypolyethoxyethanol, polyethylene glycol or octylphenoxypolyethoxyethanol. Also suitable are fatty acid esters of polyoxyethylene sorbitan, such as polyoxyethylene sorbitan trioleate.

The cationic surfactants are, especially, quarternary ammonium salts which generally have at least one alkyl radical of approximately 8 to approximately 22 C atoms as substituents and as further substituents (unhalogenated or halogenated) lower alkyl or hydroxyalkyl or benzyl radicals. The salts are preferably in the form of halides, methylsulfates or ethylsulfates. Examples are stearyltrimethylammonium chloride and benzylbis(2-chloroethyl)ethyhammonium bromide.

Examples of suitable anionic surfactants are water-soluble soaps or water-soluble synthetic surface-active compounds. Examples of suitable soaps are the alkali, alkaline earth or (unsubstituted or substituted) ammonium salts of fatty acids having approximately 10 to approximately 22 C atoms, such as the sodium or potassium salts of oleic or stearic acid, or of natural fatty acid mixtures which are obtainable for example from coconut or tall oil; mention must also be made of the fatty acid methyl taurates. However, synthetic surfactants are used more frequently, in particular fatty sulfonates, fatty sulfates, sulfonated benzimidazole derivatives or alkylaryl sulfonates. As a rule, the fatty sulfonates and fatty sulfates are present as alkali, alkaline earth or (substituted or unsubstituted) ammonium salts and they generally have an alkyl radical of approximately 8 to approximately 22 C atoms, alkyl also to be understood as including the alkyl moiety of acyl radicals; examples which may be mentioned are the sodium or calcium salts of lignosulfonic acid, of the dodecylsulfuric ester or of a fatty alcohol sulfate mixture prepared from natural fatty acids. This group also includes the salts of the sulfuric esters and sulfonic acids of fatty alcohol/ethylene oxide adducts. The sulfonated benzimidazole derivatives preferably contain 2 sulfonyl groups and a fatty acid radical of approximately 8 to approximately 22 C atoms. Examples of alkylarylsulfonates are the sodium, calcium or triethanolammonium salts of decylbenzenesulfonic acid, of dibutylnaphthalenesulfonic acid or of a naphthalenesulfonic acid/formaldehyde condensate. Also possible are, furthermore, suitable phosphates, such as salts of the phosphoric ester of a p-nonylphenol/(4-14)ethylene oxide adduct, or phospholipids. Further suitable phosphates are tris-esters of phosphoric acid with aliphatic or aromatic alcohols and/or bis-esters of alkyl phosphonic acids with aliphatic or aromatic alcohols, which are a high performance oil-type adjuvant. These tris-esters have been described, for example, in WO0147356, WO0056146, EP-A-0579052 or EP-A-1018299 or are commercially available under their chemical name. Preferred tris-esters of phosphoric acid for use in the new compositions are tris-(2-ethylhexyl) phosphate, tris-n-octyl phosphate and tris-butoxyethyl phosphate, where tris-(2-ethylhexyl) phosphate is most preferred. Suitable bis-ester of alkyl phosphonic acids are bis-(2-ethylhexyl)-(2-ethylhexyl)-phosphonate, bis-(2-ethylhexyl)-(n-octyl)-phosphonate, dibutyl-butyl phosphonate and bis(2-ethylhexyl)-tripropylene-phosphonate, where bis-(2-ethylhexyl)-(n-octyl)-phosphonate is particularly preferred.

The compositions according to the invention can preferably additionally include an additive comprising an oil of vegetable or animal origin, a mineral oil, alkyl esters of such oils or mixtures of such oils and oil derivatives. The amount of oil additive used in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil such as ADIGOR® and MERO®, olive oil or sunflower oil, emulsified vegetable oil, such as AMIGO® (Rhone-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈-C₂₂ fatty acids, especially the methyl derivatives of C₁₂-C₁₈ fatty acids, for example the methyl esters of lauric acid, palmitic acid and oleic acid, being important. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000. Also, alkoxylated fatty acids can be used as additives in the inventive compositions as well as polymethylsiloxane based additives, which have been described in WO08/037,373.

The application and action of the oil additives can be further improved by combining them with surface-active substances, such as non-ionic, anionic or cationic surfactants. Examples of suitable anionic, non-ionic and cationic surfactants are listed on pages 7 and 8 of WO 97/34485. Preferred surface-active substances are anionic surfactants of the dodecyl-benzylsulfonate type, especially the calcium salts thereof, and also non-ionic surfactants of the fatty alcohol ethoxylate type. Special preference is given to ethoxylated C₁₂-C₂₂ fatty alcohols having a degree of ethoxylation of from 5 to 40. Examples of commercially available surfactants are the Genapol types (Clariant AG). Also preferred are silicone surfactants, especially polyalkyl-oxide-modified heptamethyltrisiloxanes, which are commercially available e.g. as Silwet L-77®, and also perfluorinated surfactants. The concentration of surface-active substances in relation to the total additive is generally from 1 to 30% by weight. Examples of oil additives that consist of mixtures of oils or mineral oils or derivatives thereof with surfactants are Edenor ME SU®, Turbocharge® (Syngenta AG, CH) and Actipron® (BP Oil UK Limited, GB).

The said surface-active substances may also be used in the formulations alone, that is to say without oil additives.

Furthermore, the addition of an organic solvent to the oil additive/surfactant mixture can contribute to a further enhancement of action. Suitable solvents are, for example, Solvesso® (ESSO) and Aromatic Solvent® (Exxon Corporation). The concentration of such solvents can be from 10 to 80% by weight of the total weight. Such oil additives, which may be in admixture with solvents, are described, for example, in US-A-4 834 908. A commercially available oil additive disclosed therein is known by the name MERGE® (BASF Corporation). A further oil additive that is preferred according to the invention is SCORE® (Syngenta Crop Protection Canada.)

In addition to the oil additives listed above, in order to enhance the activity of the compositions according to the invention it is also possible for formulations of alkylpyrrolidones, (e.g. Agrimax®) to be added to the spray mixture. Formulations of synthetic latices, such as, for example, polyacrylamide, polyvinyl compounds or poly-1-p-menthene (e.g. Bond®, Courier® or Emerald®) can also be used. Solutions that contain propionic acid, for example Eurogkem Pen-e-trate®, can also be mixed into the spray mixture as activity-enhancing agents.

As a rule, the compositions comprise 0.1 to 99%, especially 0.1 to 95%, of active ingredient of the formula I and 1 to 99.9%, especially 5 to 99.9%, of at least one solid or liquid adjuvant, it being possible as a rule for 0 to 25%, especially 0.1 to 20%, of the composition to be surfactants (% in each case meaning percent by weight). Whereas concentrated compositions tend to be preferred for commercial goods, the end consumer as a rule uses dilute compositions which have substantially lower concentrations of active ingredient. Preferred compositions are composed in particular as follows (%=percent by weight):

### Emulsifiable Concentrates:

active ingredient: 1 to 95%, preferably 5 to 50%, more preferably 5 to 20%
surfactant: 1 to 30%, preferably 10 to 20%
solvent: 5 to 98%, preferably 70 to 85%

### Dusts:

active ingredient: 0.1 to 10%, preferably 2 to 5%,
solid carrier: 99.9 to 90%, preferably 99.9 to 99%

### Suspension Concentrates:

active ingredient: 5 to 75%, preferably 10 to 50%, more preferably 10 to 40%
water: 94 to 24%, preferably 88 to 30%
surfactant: 1 to 40%, preferably 2 to 30%

### Oil-Based Suspension Concentrates:

active ingredient: 2 to 75%, preferably 5 to 50%, more preferably 10 to 25%
oil: 94 to 24%, preferably 88 to 30%
surfactant: 1 to 40%, preferably 2 to 30%

### Wettable Powders:

active ingredient: 0.5 to 90%, preferably 1 to 80%, more preferably 25 to 75%
surfactant: 0.5 to 20%, preferably 1 to 15%
solid carrier: 5 to 99%, preferably 15 to 98%

### Granulates:

active ingredient: 0.5 to 30%, preferably 3 to 25%, more preferably 3 to 15%
solid carrier: 99.5 to 70%, preferably 97 to 85%

Preferably, the term “active ingredient” refers to one of the compounds of formula I. It also refers to mixtures of the compound of formula I with other insecticides, fungicides, herbicides, safeners, adjuvants and the like, which mixtures are specifically disclosed below.

The compositions can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers; fertilizers, in particular nitrogen containing fertilizers such as ammonium nitrates and urea as described in WO08/017,388, which can enhance the efficacy of the inventive compounds; or other active ingredients for achieving specific effects, for example ammonium or phosphonium salts, in particular halides, (hydrogen)sulphates, nitrates, (hydrogen)carbonates, citrates, tartrates, formiates and acetates, as described in WO07/068,427 and WO07/068,428, which also can enhance the efficacy of the inventive compounds and which can be used in combination with penetration enhancers such as alkoxalated fatty acids; bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the above-mentioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compositions according to the invention are also suitable for the protection of plant propagation material, for example seeds, such as fruit, tubers or kernels, or nursery plants, against pests of the abovementioned type. The propagation material can be treated with the compositions prior to planting, for example seed can be treated prior to sowing. Alternatively, the compositions can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention.

Further methods of application of the compositions according to the invention comprise drip application onto the soil, dipping of parts of plants such as roots bulbs or tubers, drenching the soil, as well as soil injection. These methods are known in the art.

In order to apply a compound of formula I as an insecticide, acaricide, nematicide or molluscicide to a pest, a locus of pest, or to a plant susceptible to attack by a pest, a compound of formula I is usually formulated into a composition which includes, in addition to the compound of formula I, a suitable inert diluent or carrier and, optionally, a formulation adjuvant in form of a surface active agent (SFA) as described herein or, for example, in EP-B-1062217. SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a compound of formula I. The composition is generally used for the control of pests such that a compound of formula I is applied at a rate of from 0.1 g to 10 kg per hectare, preferably from 1 g to 6 kg per hectare, more preferably from 1 g to 1 kg per hectare.

When used in a seed dressing, a compound of formula I is used at a rate of 0.0001 g to 10 g (for example 0.001 g or 0.05 g), preferably 0.005 g to 10 g, more preferably 0.005 g to 4 g, per kilogram of seed.

In another aspect the present invention provides an insecticidal, acaricidal, nematicidal or molluscicidal composition comprising an insecticidally, acaricidally, nematicidally or molluscicidally effective amount of a compound of formula I and a suitable carrier or diluent therefor.

In a still further aspect the invention provides a method of combating and controlling pests at a locus which comprises treating the pests or the locus of the pests with an insecticidally, acaricidally, nematicidally or molluscicidally effective amount of a composition comprising a compound of formula I.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), oil-based suspension concentrate (OD), aerosols, fogging/smoke formulations, capsule suspensions (CS) and seed treatment formulations. The formulation type chosen in any instance will depend upon the particular purpose en-visaged and the physical, chemical and biological properties of the compound of formula I.

Dustable powders (DP) may be prepared by mixing a compound of formula I with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing a compound of formula I with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula I with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula I and one or more powdered solid diluents or carriers, or from pre-formed blank granules by absorbing a compound of formula I (or a solution thereof, in a suitable agent) in a porous granular material (such as pumice, attapulgite clays, fuller's earth, kieselguhr, diatomaceous earths or ground corn cobs) or by adsorbing a compound of formula I (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula I in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of formula I in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula I either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of formula I is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula I. SCs may be prepared by ball or bead milling the solid compound of formula I in a suitable medium, optionally with one or more dispersing agents, to produce a fine particle suspension of the compound. One or more wetting agents may be included in the composition and a suspending agent may be included to reduce the rate at which the particles settle. Alternatively, a compound of formula I may be dry milled and added to water, containing agents hereinbefore described, to produce the desired end product.

Oil-based suspension concentrate (OD) may be prepared similarly by suspending finely divided insoluble solid particles of a compound of formula I in an organic fluid (for example at least one mineral oil or vegetable oil). ODs may further comprise at least one penetration promoter (for example an alcohol ethoxylate or a related compound), at least one non-ionic surfactants and/or at least one anionic surfactant, and optionally at least one additive from the group of emulsifiers, foam-inhibiting agents, preservatives, anti-oxidants, dyestuffs, and/or inert filler materials. An OD is intended and suitable for dilution with water before use to produce a spray solution with sufficient stability to allow spray application through appropriate equipment.

Aerosol formulations comprise a compound of formula I and a suitable propellant (for example n-butane). A compound of formula I may also be dissolved or dispersed in a suitable medium (for example water or a water miscible liquid, such as n-propanol) to provide compositions for use in non-pressurised, hand-actuated spray pumps.

A compound of formula I may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of formula I and, optionally, a carrier or diluent therefor. The polymeric shell may be produced by either an interfacial polycondensation reaction or by a coacervation procedure. The compositions may provide for controlled release of the compound of formula I and they may be used for seed treatment. A compound of formula I may also be formulated in a biodegradable polymeric matrix to provide a slow, controlled release of the compound.

A compound of formula I may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC, OD and DC compositions described above. Compositions for treating seed may include an agent for assisting the adhesion of the composition to the seed (for example a mineral oil or a film-forming barrier).

A composition of the present invention may include one or more additives to improve the biological performance of the composition (for example by improving wetting, retention or distribution on surfaces; resistance to rain on treated surfaces; or uptake or mobility of a compound of formula I). Such additives include surface active agents (SFAs), spray additives based on oils, for example certain mineral oils, vegetable oils or natural plant oils (such as soy bean and rape seed oil), and blends of these with other bio-enhancing adjuvants (ingredients which may aid or modify the action of a compound of formula I). Increasing the effect of a compound of formula I may for example be achieved by adding ammonium and/or phosphonium salts, and/or optionally at least one penetration promotor such as fatty alcohol alkoxylates (for example rape oil methyl ester) or vegetable oil esters.

Wetting agents, dispersing agents and emulsifying agents may be surface active agents (SFAs) of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of formula I may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

A compound of formula I may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ODs, ECs, EWs, MEs SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula I (for example 0.0001 to 10%, by weight) depending upon the purpose for which they are to be used.

A compound of formula I may be used in mixtures with fertilisers (for example nitrogen-, potassium- or phosphorus-containing fertilisers, and more particularly ammonium nitrate and/or urea fertilizers). Suitable formulation types include granules of fertiliser. The mixtures suitably contain up to 25% by weight of the compound of formula I.

The invention therefore also provides a fertiliser composition comprising a fertiliser and a compound of formula I.

The compositions of this invention may contain other compounds having biological activity, for example micronutrients or compounds having fungicidal activity or which possess plant growth regulating, herbicidal, safening, insecticidal, nematicidal or acaricidal activity.

The compound of formula I may be the sole active ingredient of the composition or it may be admixed with one or more additional active ingredients such as a pesticide (insect, acarine, mollusc and nematode pesticide), fungicide, synergist, herbicide, safener or plant growth regulator where appropriate. The activity of the compositions according to the invention may thereby be broadened considerably and may have surprising advantages which can also be described, in a wider sense, as synergistic activity. An additional active ingredient may: provide a composition having a broader spectrum of activity or increased persistence at a locus; provide a composition demonstrating better plant/crop tolerance by reducing phytotoxicity; provide a composition controlling insects in their different development stages; synergise the activity or complement the activity (for example by increasing the speed of effect or overcoming repellency) of the compound of formula I; or help to overcome or prevent the development of resistance to individual components. The particular additional active ingredient will depend upon the intended utility of the composition. Examples of suitable pesticides include the following:

a) Pyrethroids, such as permethrin, cypermethrin, fenvalerate, esfenvalerate, deltamethrin, cyhalothrin (in particular lambda-cyhalothrin), bifenthrin, fenpropathrin, cyfluthrin, tefluthrin, fish safe pyrethroids (for example ethofenprox), natural pyrethrin, tetramethrin, s-bioallethrin, fenfluthrin, prallethrin or 5-benzyl-3-furylmethyl-(E)-(1R,3S)-2,2-dimethyl-3-(2-oxothiolan-3-ylidenemethyl)cyclopropane carboxylate;
b) Organophosphates, such as, profenofos, sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate or diazinon;
c) Carbamates (including aryl carbamates), such as pirimicarb, triazamate, cloethocarb, carbofuran, furathiocarb, ethiofencarb, aldicarb, thiofurox, carbosulfan, bendiocarb, fenobucarb, propoxur, methomyl or oxamyl;
d) Benzoyl ureas, such as diflubenzuron, triflumuron, hexaflumuron, flufenoxuron or chlorfluazuron;
e) Organic tin compounds, such as cyhexatin, fenbutatin oxide or azocyclotin;
f) Pyrazoles, such as tebufenpyrad and fenpyroximate;
g) Macrolides, such as avermectins or milbemycins, for example abamectin, emamectin benzoate, ivermectin, milbemycin, or spinosad, spinetoram or azadirachtin;
h) Hormones or pheromones;
i) Organochlorine compounds such as endosulfan, benzene hexachloride, DDT, chlordane or dieldrin;
j) Amidines, such as chlordimeform or amitraz;
k) Fumigant agents, such as chloropicrin, dichloropropane, methyl bromide or metam;
l) Neonicotinoid compounds such as imidacloprid, thiacloprid, acetamiprid, clothianidin, nitenpyram, dinotefuran or thiamethoxam;
m) Diacylhydrazines, such as tebufenozide, chromafenozide or methoxyfenozide;
n) Diphenyl ethers, such as diofenolan or pyriproxifen;
o) indoxacarb;

### p) Chlorfenapyr;

q) Pymetrozine or pyrifluquinazon;
r) Spirotetramat, spirodiclofen or spiromesifen;
s) Flubendiamide, chloranthraliniprole, or cyanthraniliprole;
t) Cyenopyrafen or cyflumetofen; or

### u) Sulfoxaflor.

In addition to the major chemical classes of pesticide listed above, other pesticides having particular targets may be employed in the composition, if appropriate for the intended utility of the composition. For instance, selective insecticides for particular crops, for example stemborer specific insecticides (such as cartap) or hopper specific insecticides (such as buprofezin) for use in rice may be employed. Alternatively insecticides or acaricides specific for particular insect species/stages may also be included in the compositions (for example acaricidal ovo-larvicides, such as clofentezine, flubenzimine, hexythiazox or tetradifon; acaricidal motilicides, such as dicofol or propargite; acaricides, such as bromopropylate or chlorobenzilate; or growth regulators, such as hydramethylnon, cyromazine, methoprene, chlorfluazuron or diflubenzuron).

In the above-mentioned mixtures of compounds of formula I with other pesticides, the mixing ratios can vary over a large range and are, preferably 100:1 to 1:6000, especially 50:1 to 1:50, more especially 20:1 to 1:20, even more especially 10:1 to 1:10. Those mixing ratios are understood to include, on the one hand, ratios by weight and also, on other hand, molar ratios.

The mixtures can advantageously be used in the above-mentioned formulations (in which case “active ingredient” relates to the respective mixture of compound of formula I with the mixing partner).

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

The mixtures comprising a compound of formula I and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I and the active ingredients as described above is not essential for working the present invention.

The following examples illustrate the invention further but do not limit the invention.

### PREPARATION EXAMPLES

Those skilled in the art will appreciate that certain compounds described below are □-ketoenols, and as such may exist as a single tautomer or as a mixture of keto-enol and diketone tautomers, as described, for example by J. March, Advanced Organic Chemistry, third edition, John Wiley and Sons. The compounds are shown in Table T1 as a single enol tautomer, but it should be inferred that this description covers both the diketone form and any possible enols which could arise through tautomerism. Furthermore, some of the compounds in Table T1 and Table P1 are drawn as single enantiomers for the purposes of simplicity, but unless specified as single enantiomers these structures should be construed as representing a mixture of enantiomers.

Within the detailed experimental section the diketone tautomer is chosen for naming purposes, even if the predominant tautomer is the enol form.

Where more than one tautomer observed in proton NMR, the data shown are for the mixture of tautomers.

### Example 1

### Preparation of 2,2-dimethyl-propionic acid 3-oxo-4-(tetrahydro-pyran-4-ylmethyl)-2-(2,4,6-trimethyl-phenyl)-cyclopent-1-enyl ester

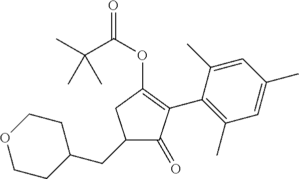 [SMILES: Cc1cc(C)c(C2=C(OC(=O)C(C)(C)C)CC(CC3CCOCC3)C2=O)c(C)c1]

### Step 1

### Preparation of 2-(2,4,6-trimethylphenyl)-3-methoxy-cyclopent-2-enone

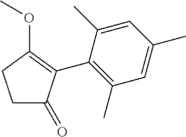

To a suspension of 2-bromo-3-methoxy-cyclopent-2-enone (6.75 g, 35.3 mmol), 2,4,6-trimethylphenyl boronic acid (6.99 g, 42.6 mmol) and freshly ground potassium phosphate (15 g, 70.6 mmol) in degassed toluene (180 ml) under nitrogen are added Pd(OAc)₂ (159 mg, 0.71 mmol) and S-Phos (579 mg, 1.41 mmol), and the reaction heated to 90° C. with stirring under N₂ for 4 hours. The reaction mixture is partitioned between ethyl acetate (150 ml) and water (150 ml), and the organic layer is removed, Silica gel is added to the organic layer, the solvent is evaporated under reduced pressure and the residue is purified by flash chromatography on silica gel to give 2-(2,4,6-trimethylphenyl)-3-methoxy-cyclopent-2-enone (6.2 g).

### Step 2

### Preparation of 5-[hydroxy-(tetrahydro-pyran-4-yl)-methyl]-3-methoxy-2-(2,4,6-trimethyl-phenyl)-cyclopent-2-enone

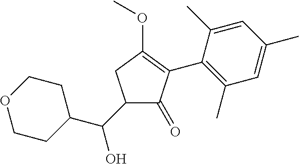 [SMILES: COC1=C(c2c(C)cc(C)cc2C)C(=O)C(C(O)C2CCOCC2)C1]

To a solution of N-ethyl-N,N-diisopropylamine (527 □l, 3.76 mmol) in THF (5 ml) under N₂ at −78° C. is added, dropwise, a 2.5M solution of butyllithium in hexane (1.32 ml, 3.3 mmol) and the reaction allowed to stir at −78° C. for 20 minutes. This pale yellow solution is then added dropwise, over a period of 10 minutes, to a solution of 2-(2,4,6-trimethylphenyl)-3-methoxy-cyclopent-2-enone (691 ml, 3 mmol) in THF (5 ml) under N₂, which is pre-cooled to −78° C. The resulting solution is allowed to stir at −78° C. for 40 minutes. A solution of tetrahydropyran-4-carbaldehyde (377 mg, 3.3 mmol) in THF (1 ml) is then added in one portion, the reaction mixture is stirred at −78° C. for 30 minutes before being allowed to warm to room temperature over a period of 60 minutes. The reaction was quenched by the addition of saturated aqueous ammonium chloride (50 ml) and extracted with ethyl acetate (2×50 ml). The combined organics were purified by flash chromatography to give 5-[hydroxy-(tetrahydro-pyran-4-yl)-methyl]-3-methoxy-2-(2,4,6-trimethyl-phenyl)-cyclopent-2-enone (648 mg).

### Step 3

### Preparation of 4-[1-(tetrahydro-pyran-4-yl)-meth-(E)-ylidene]-2-(2,4,6-trimethyl-phenyl)-cyclopentane-1,3-dione

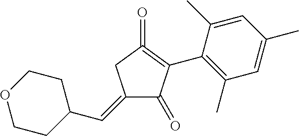

To a solution of 5-[hydroxy-(tetrahydro-pyran-4-yl)-methyl]-3-methoxy-2-(2,4,6-trimethyl-phenyl)-cyclopent-2-enone (408 mg, 1.18 mmol) in acetone (2 ml) is added a 2N solution of hydrochloric acid (2 ml) and the resulting solution is heated to 130° C. by microwave irradiation for 90 minutes. The reaction mixture was diluted with 2N hydrochloric acid (25 ml), and extracted with ethyl acetate (2×25 ml). The combined organics are washed with brine (25 ml), dried over magnesium sulphate, filtered and concentrated in vacuo to give 441-(tetrahydro-pyran-4-yl)-meth-(E)-ylidene]-2-(2,4,6-trimethyl-phenyl)-cyclopentane-1,3-dione (302 mg).

### Step 4

### Preparation of 4-(tetrahydro-pyran-4-ylmethyl)-2-(2,4,6-trimethyl-phenyl)-cyclopentane-1,3-dione

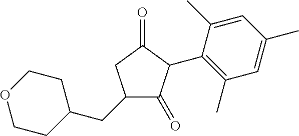 [SMILES: Cc1cc(C)c(C2C(=O)CC(CC3CCOCC3)C2=O)c(C)c1]

To a solution of 4-[1-(tetrahydro-pyran-4-yl)-meth-(E)-ylidene]-2-(2,4,6-trimethyl-phenyl)-cyclopentane-1,3-dione (270 mg, 0.86 mmol) in ethanol (10 ml) was added 10% palladium on charcoal (27 mg) and the resulting solution stirred under hydrogen (3 bar) for 5 hours at room temperature. The reaction mixture was then filtered through a pad of celite, which was washed with methanol, and the filtrated concentrated in vacuo to give 4-(tetrahydro-pyran-4-ylmethyl)-2-(2,4,6-trimethyl-phenyl)-cyclopentane-1,3-dione (258 mg).

### Step 5

### Preparation of 2,2-dimethyl-propionic acid 3-oxo-4-(tetrahydro-pyran-4-ylmethyl)-2-(2,4,6-trimethyl-phenyl)-cyclopent-1-enyl ester

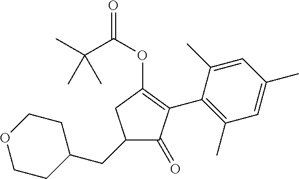 [SMILES: Cc1cc(C)c(C2=C(OC(=O)C(C)(C)C)CC(CC3CCOCC3)C2=O)c(C)c1]

To a solution of 4-(tetrahydro-pyran-4-ylmethyl)-2-(2,4,6-trimethyl-phenyl)-cyclopentane-1,3-dione (100 mg, 0.25 mmol) in dichloromethane (5 ml) and triethylamine (140 □l, 1 mmol) is added the pivaloyl chloride (91 □l, 1 mmol) at room temperature. The reaction mixture is stirred overnight at room temperature. Silica gel is added to the crude reaction mixture, the solvent is evaporated under reduced pressure and the residue is purified by flash chromatography on silica gel to give 2,2-dimethyl-propionic acid 3-oxo-4-(tetrahydro-pyran-4-ylmethyl)-2-(2,4,6-trimethyl-phenyl)-cyclopent-1-enyl ester (102 mg).

### Example 2

### Preparation of 2-(3,5-dimethylbiphenyl-4-yl)-4-(tetrahydrofuran-3-ylmethyl)cyclopentane-1,3-dione

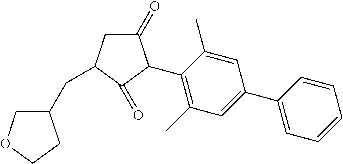 [SMILES: Cc1cc(-c2ccccc2)cc(C)c1C1C(=O)CC(CC2CCOC2)C1=O]

### Step 1

### Preparation of 2-(3,5-dimethylbiphenyl-4-yl)-3-methoxy cyclopent-2-enone

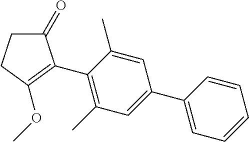 [SMILES: COC1=C(c2c(C)cc(-c3ccccc3)cc2C)C(=O)CC1]

To a mixture of 2-(4-bromo-2,6-dimethylphenyl)-3-methoxy-cyclopent-2-enone (1 g, 3.4 mmol), cesium fluoride (1.5 g, 9.87 mmol), phenylboronic acid (0.5 g, 4.1 mmol) and [1,1′-bis(diphenylphosphino)ferrocene]dichloro-palladium(II) (0.44 g, 0.54 mmol) is added degassed dimethoxyethane (10 ml) and the resulting suspension is stirred under nitrogen for 45 minutes then heated at 80° C. for 4 hrs. After cooling to room temperature the reaction mixture is acidified with 1N aqueous hydrochloric acid. The aqueous phase is further extracted with ethyl acetate (3×100 ml) and then all organic fractions are combined, dried over anhydrous sodium sulphate and concentrated in vacuo. The resulting material is purified by column chromatography on silica gel to afford 2-(3,5-dimethylbiphenyl-4-yl)-3-methoxy cyclopent-2-enone (0.7 g) as a white solid.

### Step 2

### Preparation of 2-(3,5-dimethylbiphenyl-4-yl)-5-[hydroxyl-(tetrahydrofuran-3-yl)-methyl]-3-methoxy-cyclopent-2-enone

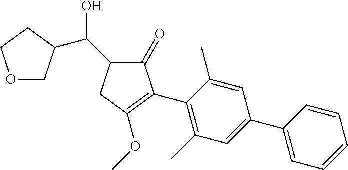 [SMILES: COC1=C(c2c(C)cc(-c3ccccc3)cc2C)C(=O)C(C(O)C2CCOC2)C1]

To a solution of 2-(3,5-Dimethylbiphenyl-4-yl)-3-methoxy cyclopent-2-enone (0.6 g, 2.05 mmol) in tetrahydrofuran (12 ml) is added 1 molar solution of lithium bis(trimethylsilyl)amide in THF (2.5 ml, 2.5 mmol) under nitrogen atmosphere at −75° C. The resulting solution is stirred at −75° C. for 40 minutes and to this mixture, a solution of 3-tetrahydrofurancarboxaldehyde (0.42 g, 4.1 mmol) in THF is added over 20 minutes. The resulting solution is stirred at −75° C. for 2 hours. The cooling bath is removed and the mixture is allowed to reach room temperature, then stirred for 2 hours. The reaction mixture is quenched with ice cold water (100 ml) and extracted with ethyl acetate (3×75 ml, dried over anhydrous sodium sulphate, filtered and the filtrate is concentrated in vacuo to give a residue (1.1 g) which is used as such for the next step.

### Step 3

### Preparation of 2-(3,5-dimethylbiphenyl-4-yl)-4-[1-(tetrahydrofuran-3-yl)methylidene]-cyclopentane-1,3-dione

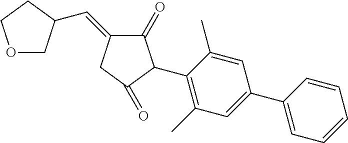 [SMILES: Cc1cc(-c2ccccc2)cc(C)c1C1C(=O)C/C(=C\C2CCOC2)C1=O]

A mixture of 2-(3,5-Dimethylbiphenyl-4-yl)-5-[hydroxyl-(tetrahydrofuran-3-yl)-methyl]-3-methoxy-cyclopent-2-enone (1.1 g, 2.8 mmol), acetone (21 ml) and 2N hydrochloric acid (10 ml) is heated under microwave conditions at 130° C. for 40 minutes. The organic solvent is evaporated under vacuo, diluted with water (100 ml) and extracted with ethyl acetate (3×100 ml). The combined organic extracts are combined, washed with water and brine, dried over anhydrous sodium sulphate, filtered and the filtrate is concentrated in vacuo. The residue is purified by column chromatography on silica gel to give 2-(3,5-dimethylbiphenyl-4-yl)-4-[1-(tetrahydrofuran-3-yl)methylidene]-cyclopentane-1,3-dione (0.29 g).

### Step 4

### Preparation of 2-(3,5-dimethylbiphenyl-4-yl)-4-(tetrahydrofuran-3ylmethyl)-cyclopentane-1,3-dione

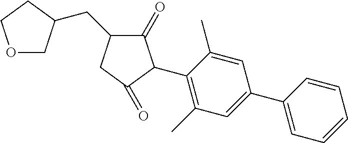 [SMILES: Cc1cc(-c2ccccc2)cc(C)c1C1C(=O)CC(CC2CCOC2)C1=O]

To a solution of 2-(3,5-dimethylbiphenyl-4-yl)-4-[1-(tetrahydrofuran-3-yl)methylidene]-cyclopentane-1,3-dione (0.29 g, 0.8 mmol) in methanol (10 ml) is added 10% palladium on carbon (0.06 g), followed by stirring under a 1 bar hydrogen atmosphere for 8 hours. The reaction mixture is then filtered through diatomaceous earth and concentrated to give a crude product which is purified by flash chromatography (hexane/ethyl acetate) to afford 2-(3,5-dimethylbiphenyl-4-yl)-4-(tetrahydrofuran-3-ylmethyl)-cyclopentane-1,3-dione (0.12 g).

### Example 3

### Preparation of 2-(4-fluoro-2,6-dimethylphenyl)-4-(tetrahydropyran-4-ylmethyl)cyclopentane-1,3-dione

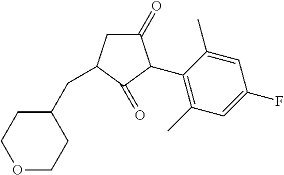 [SMILES: Cc1cc(F)cc(C)c1C1C(=O)CC(CC2CCOCC2)C1=O]

### Step 1

### Preparation of (4-Fluoro-2,6-dimethylphenyl)furan-2-ylmethanol

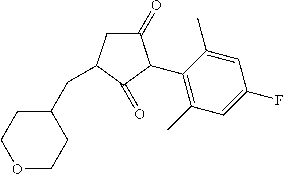 [SMILES: Cc1cc(F)cc(C)c1C1C(=O)CC(CC2CCOCC2)C1=O]

To a solution of 5-fluoro-2-iodo-1,3-xylene (11 g, 44 mmol) in tetrahydrofuran (110 ml) is added 1.6 molar solution of n-butyl lithium in hexane (33 ml, 52 mmol) under nitrogen atmosphere at −75° C. The resulting solution is stirred at −75° C. for 60 minutes and to this mixture, a solution of furfural (6.3 g, 65.6 mmol) in THF (20 ml) is added over 20 minutes. The resulting solution is stirred at −75° C. for 2 hours. The cooling bath is removed and the mixture is allowed to reach room temperature, and then stirred for 5 hours. The reaction mixture is quenched with ice cold water (1000 ml) and extracted with ethyl acetate (3×250 ml), dried over anhydrous sodium sulphate, filtered and the filtrate is concentrated in vacuo to give a residue which is purified by flash chromatography (hexane/ethyl acetate) to afford (4-Fluoro-2,6-dimethylphenyl)furan-2-ylmethanol (6 g).

### Step 2

### Preparation of 5-(4-Fluoro-2,6-dimethylphenyl)-4-hydroxy cyclopent-2-enone

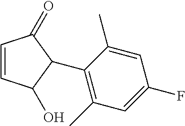 [SMILES: Cc1cc(F)cc(C)c1C1C(=O)C=CC1O]

A mixture of (4-fluoro-2,6-dimethylphenyl)furan-2-ylmethanol (6 g, 27 mmol), acetone (150 ml), water (24 ml) and orthophosphoric acid (0.6 ml) is heated under microwave conditions at 120° C. for 50 minutes. The organic solvent is evaporated under vacuo, diluted with water (150 ml) and extracted with ethylacetate (3×100 ml). The combined organic extracts are combined, washed with water and brine, dried over anhydrous sodium sulphate, filtered and the filtrate is concentrated in vacuo. The residue is purified by column chromatography on silica gel to give 5-(4-fluoro-2,6-dimethylphenyl)-4-hydroxy cyclopent-2-enone (3 g).

### Step 3

### Preparation of 2-(4-Fluoro-2,6-dimethylphenyl)cyclopent-4-ene-1,3-dione

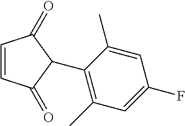 [SMILES: Cc1cc(F)cc(C)c1C1C(=O)C=CC1=O]

To a solution of 5-(4-fluoro-2,6-dimethylphenyl)-4-hydroxy cyclopent-2-enone (3 g, 13.6 mmol) in acetone (36 ml) is added freshly prepared Jones reagent (24 ml) at 0° C. The resulting solution is stirred at 0° C. for 60 minutes. The reaction mixture is quenched with ice cold isopropyl alcohol (25 ml) and stirred for one hour. The organics evaporated under vacuo and extracted with ethyl acetate, dried over anhydrous sodium sulphate, filtered and the filtrate is concentrated in vacuo to give a residue which is purified by flash chromatography (hexane/ethyl acetate) to afford 2-(4-fluoro-2,6-dimethylphenyl)cyclopent-4-ene-1,3-dione (2.9 g).

### Step 4

### Preparation of 2-(4-fluoro-2,6-dimethylphenyl)-cyclopentane-1,3-dione

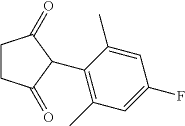 [SMILES: Cc1cc(F)cc(C)c1C1C(=O)CCC1=O]

To a solution of 2-(4-fluoro-2,6-dimethylphenyl)cyclopent-4-ene-1,3-dione (2.9 g, 13.3 mmol) in acetic acid (116 ml) is added zinc powder (6 g, 91.7 mmol) at 25-30° C. The resulting solution is stirred at 25-30° C. for 16 hours. The reaction mixture is then filtered through diatomaceous earth and concentrated to give a crude product (2.9 g) which is used for the next step.

### Step 5

### Preparation of 2-(4-fluoro-2,6-dimethylphenyl)-3-methoxy-cyclopent-2-enone

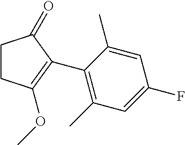 [SMILES: COC1=C(c2c(C)cc(F)cc2C)C(=O)CC1]

To a solution of 2-(4-fluoro-2,6-dimethylphenyl)-cyclopentane-1,3-dione (2.9 g, 13.3 mmol) in tetrahydrofuran (290 ml) is added anhydrous potassium carbonate (22 g, 159 mmol) and iodomethane (22.6 g, 159 mmol. The resulting mixture is stirred at 25-30° C. for 16 hours. The organic layer is evaporated, reaction mixture is quenched with water (150 ml) and extracted with ethylacetate (3×100 ml). The combined organic extracts are combined, washed with water and brine, dried over anhydrous sodium sulphate, filtered and the filtrate is concentrated in vacuo. The residue is purified by column chromatography on silica gel to give 2-(4-fluoro-2,6-dimethylphenyl)-3-methoxy-cyclopent-2-enone (2 g).

### Step 6

### Preparation of 2-(4-fluoro-2,6-dimethylphenyl)-3-methoxy-5-[1-tetrahydropyran-4-yl)-ethyl]-cyclopent-2-enone

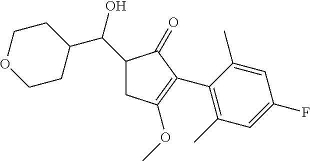 [SMILES: COC1=C(c2c(C)cc(F)cc2C)C(=O)C(C(O)C2CCOCC2)C1]

To a solution of 2-(4-fluoro-2,6-dimethylphenyl)-3-methoxy-cyclopent-2-enone (0.5 g, 2.1 mmol) in tetrahydrofuran (10 ml) is added 1 molar solution of lithium bis(trimethylsilyl)amide in THF (2.7 ml) under nitrogen atmosphere at −75° C. The resulting solution is stirred at −75° C. for 40 minutes and to this mixture, a solution of 3-tetrahydrofurancarboxaldehyde (0.5 g, 4.38 mmol) in THF is added over 20 minutes. The resulting solution is stirred at −75° C. for 2 hours. The cooling bath is removed and the mixture is allowed to reach room temperature and then stirred for 2 hours. The reaction mixture is quenched with ice cold water (100 ml) and extracted with ethyl acetate (3×75 ml), dried over anhydrous sodium sulphate, filtered and the filtrate is concentrated in vacuo to give a residue (0.7 g) which is used as such for the next step.

### Step 7

### Preparation of 2-(4-fluoro-2,6-dimethylphenyl)-4-[1-(tetrahydropyran-4-yl)-methylidene]-cyclopentane-1,3-dione

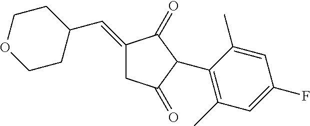 [SMILES: Cc1cc(F)cc(C)c1C1C(=O)C/C(=C\C2CCOCC2)C1=O]

A mixture of 2-(4-fluoro-2,6-dimethylphenyl)-3-methoxy-54-[1-tetrahydropyran-4-yl)-ethyl]-cyclopent-2-enone (0.7 g, 2 mmol), acetone (10 ml) and 2N hydrochloric acid (10 ml) is heated under microwave conditions at 130° C. for 40 minutes. The organic solvent is evaporated under vacuo, diluted with water (100 ml) and extracted with ethylacetate (3×75 ml). The combined organic extracts are combined, washed with water and brine, dried over anhydrous sodium sulphate, filtered and the filtrate is concentrated in vacuo. The residue is purified by column chromatography on silica gel to give 2-(4-fluoro-2,6-dimethylphenyl)-4-[1-(tetrahydropyran-4-yl)-methylidene]-cyclopentane-1,3-dione (0.23 g).

### Step 8

### Preparation of 2-(4-fluoro-2,6-dimethylphenyl)-4-[tetrahydropyran-4-ylmethyl)-cyclopentane-1,3-dione

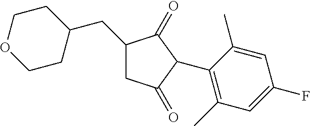 [SMILES: Cc1cc(F)cc(C)c1C1C(=O)CC(CC2CCOCC2)C1=O]

To a solution of 2-(4-fluoro-2,6-dimethylphenyl)-4-[1-(tetrahydropyran-4-yl)-methylidene]-cyclopentane-1,3-dione (0.14 g, 0.44 mmol) in methanol (3 ml) is added 10% palladium on carbon (1.5 mg), followed by stirring under a 1 bar hydrogen atmosphere for 8 hours. The reaction mixture is then filtered through diatomaceous earth and concentrated to give a crude product which is purified by flash chromatography (hexane/ethyl acetate) to afford 2-(4-fluoro-2,6-dimethylphenyl)-4-[tetrahydropyran-4-ylmethyl)cyclopentane-1,3-dione (0.13 M.

### Example 3

### Preparation of 2-(4-bromo-2,6-dimethylphenyl)-4-(tetrahydropyran-4-ylmethyl)cyclopentane-1,3-dione

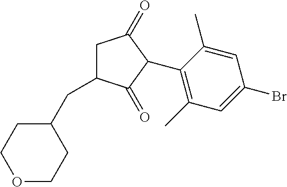 [SMILES: Cc1cc(Br)cc(C)c1C1C(=O)CC(CC2CCOCC2)C1=O]

To a solution of 2-(4-bromo-2,6-dimethylphenyl)-4-[1-(tetrahydropyran-4-yl)-methylidene]-cyclopentane-1,3-dione (0.1 g, 0.26 mmol) in methanol (100 ml) is subjected to hydrogenation under H-Cube conditions using 10% platinum carbon under a 20 bar hydrogen atmosphere. The reaction mixture is then concentrated to give a crude product which is purified by flash chromatography (hexane/ethyl acetate) to afford 2-(4-bromo-2,6-dimethylphenyl)-4-(tetrahydropyran-4-ylmethyl)cyclopentane-1,3-dione (0.09 g).

### Example 4

### Preparation of 2-(2,6-dimethylphenyl)-4-(tetrahydropyran-4-ylmethyl)cyclopentane-1,3-dione

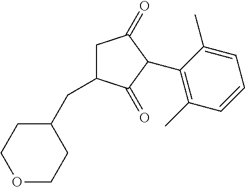 [SMILES: Cc1cccc(C)c1C1C(=O)CC(CC2CCOCC2)C1=O]

To a solution of 2-(4-bromo-2,6-dimethylphenyl)-4-[1-(tetrahydropyran-4-yl)-methylidene]-cyclopentane-1,3-dione (0.3 g, 0.8 mmol) in methanol (5 ml) is added 10% palladium on carbon (0.06 g), followed by stirring under a 1 bar hydrogen atmosphere for 8 hours. The reaction mixture is then filtered through diatomaceous earth and concentrated to give a crude product which is purified by flash chromatography (hexane/ethyl acetate) to afford 2-(2,6-dimethylphenyl)-4-(tetrahydropyran-4-ylmethyl)cyclopentane-1,3-dione (0.12 g).

### Example 5

### Preparation of 2-(4-cyclopropyl-2,6-dimethyl-phenyl)-4-(tetrahydropyran-4-ylmethyl)-cyclopentane-1,3-dione

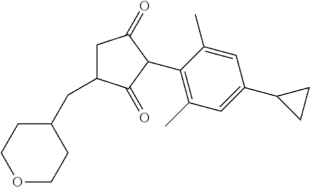 [SMILES: Cc1cc(C2CC2)cc(C)c1C1C(=O)CC(CC2CCOCC2)C1=O]

A mixture of 2-(4-bromo-2,6-dimethylphenyl)-4-(tetrahydropyran-4-ylmethyl)cyclopentane-1,3-dione (0.1 g, 0.26 mmol), potassium phosphate (0.11 g, 0.53 mmol), cyclopropyl boronic acid (0.09 g, 1.05 mmol) and tetrakis(triphenylphosphine)palladium (0) (0.06 g, 0.053 mmol), toluene (2 ml), dimethoxyethane (0.5 ml) and water (0.5 ml) is heated under microwave conditions at 130° C. for 22 minutes. The organic solvent is evaporated under vacuo, diluted with water and extracted with ethylacetate (3×25 ml). The combined organic extracts are combined, washed with water and brine, dried over anhydrous sodium sulphate, filtered and the filtrate is concentrated in vacuo. The residue is purified by column chromatography on silica gel followed by preparative HPLC to give 2-(4-cyclopropyl-2,6-dimethyl-phenyl)-4-(tetrahydropyran-4-ylmethyl)-cyclopentane-1,3-dione (0.012 g).

### Example 6

### Preparation of (4-(1-cyclopropanecarbonyl-piperidin-4-ylmethyl)-2-(2,4,6-trimethyl-phenyl)-cyclopentane-1,3-dione

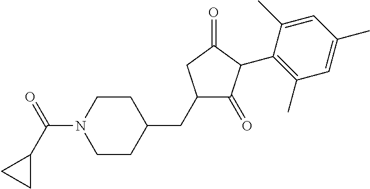 [SMILES: Cc1cc(C)c(C2C(=O)CC(CC3CCN(C(=O)C4CC4)CC3)C2=O)c(C)c1]

### Step 1

### Preparation of 4-[4-methoxy-2-oxo-3-(2,4,6-trimethyl-phenyl)-cyclopent-3-en-(E)-ylidenemethyl]-piperidine-1-carboxylic acid tert-butyl ester

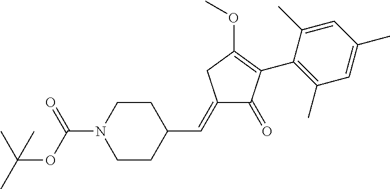 [SMILES: COC1=C(c2c(C)cc(C)cc2C)C(=O)/C(=C/C2CCN(C(=O)OC(C)(C)C)CC2)C1]

To a solution of 2-(2,4,6-trimethylphenyl)-3-methoxy-cyclopent-2-enone (9.05 g, 39.21 mmol) in THF (150 ml) under N₂ at −78° C. is added, dropwise over a period of 30 minutes, lithium diisopropylamide solution (24 ml, 43.24 mmol, 1.8 M in hexane/THF/ethylbenzene), and the reaction allowed to stir at this temperature for a further 30 minutes. 4-Formyl-piperidine-1-carboxylic acid tert-butyl ester (10 g, 43.24 mmol) is then added in one portion and the reaction kept at −78° C. for 30 minutes, before being allowed to warm gradually to room temperature over a period of 60 minutes. Potassium tert-butoxide (7.28 g, 64.86 mmol) is then added in one portion and the reaction stirred at room temperature for a further 2 hours. The reaction is quenched by the addition of saturated aqueous ammonium chloride solution (500 ml), and extracted with ethyl acetate (500 ml). The organic layer is removed, silica gel is added to the organic layer, the solvent is evaporated under reduced pressure and the residue is purified by flash chromatography on silica gel to give 4-[4-methoxy-2-oxo-3-(2,4,6-trimethyl-phenyl)-cyclopent-3-en-(E)-ylidenemethyl]-piperidine-1-carboxylic acid tert-butyl ester (15.33 g)

### Step 2

### Preparation of 4-[4-methoxy-2-oxo-3-(2,4,6-trimethyl-phenyl)-cyclopent-3-enylmethyl]-piperidine-1-carboxylic acid tert-butyl ester

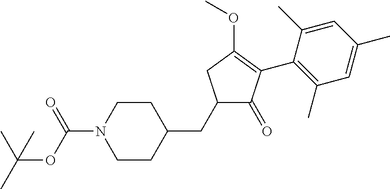 [SMILES: COC1=C(c2c(C)cc(C)cc2C)C(=O)C(CC2CCN(C(=O)OC(C)(C)C)CC2)C1]

To a solution of 4-[4-methoxy-2-oxo-3-(2,4,6-trimethyl-phenyl)-cyclopent-3-en-(E)-ylidenemethyl]-piperidine-1-carboxylic acid tert-butyl ester (15.33 g, 36.02 mmol) in ethanol (150 ml) is added 10% palladium on activated charcoal (1.53 g) and the reaction stirred under hydrogen (4 bar) for hours. The reaction is filtered through a pad of Celite and the solvent removed under reduced pressure to give 4-[4-methoxy-2-oxo-3-(2,4,6-trimethyl-phenyl)-cyclopent-3-enylmethyl]-piperidine-1-carboxylic acid tert-butyl ester (15.4 g)

### Step 3

### Preparation of 4-[2,4-dioxo-3-(2,4,6-trimethyl-phenyl)-cyclopentylmethyl]-piperidinium hydrochloride

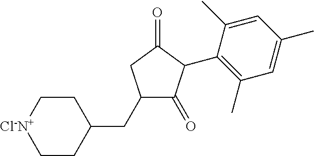

To a solution of 4-[4-methoxy-2-oxo-3-(2,4,6-trimethyl-phenyl)-cyclopent-3-enylmethyl]-piperidine-1-carboxylic acid tert-butyl ester (15.4 g, 36 mmol) in acetone (100 ml) is added 2N HCl (100 ml) and the reaction heated to reflux for 4 hours. The solvent is removed under reduced pressure to give 4-[2,4-dioxo-3-(2,4,6-trimethyl-phenyl)-cyclopentylmethyl]-piperidinium hydrochloride (12.58 g)

### Step 4

### Preparation of cyclopropanecarboxylic acid 4-(1-cyclopropanecarbonyl-piperidin-4-ylmethyl)-3-oxo-2-(2,4,6-trimethyl-phenyl)-cyclopent-1-enyl ester

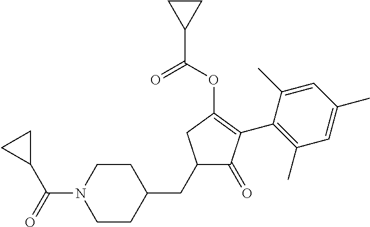 [SMILES: Cc1cc(C)c(C2=C(OC(=O)C3CC3)CC(CC3CCN(C(=O)C4CC4)CC3)C2=O)c(C)c1]

To a suspension of 4-[2,4-dioxo-3-(2,4,6-trimethyl-phenyl)-cyclopentylmethyl]-piperidinium hydrochloride (175 mg, 0.5 mmol) in DCM (5 ml) is added triethylamine (697 □l, 5 mmol), followed by cyclopropyl carbonyl chloride (608 □l, 4.5 mmol) and the reaction stirred at room temperature for 5 hours. Silica gel is added to the crude reaction, the solvent is evaporated under reduced pressure and the residue is purified by flash chromatography on silica gel to give cyclopropanecarboxylic acid 4-(1-cyclopropanecarbonyl-piperidin-4-ylmethyl)-3-oxo-2-(2,4,6-trimethyl-phenyl)-cyclopent-1-enyl ester (162 mg)

### Step 5

### Preparation of 4-(1-cyclopropanecarbonyl-piperidin-4-ylmethyl)-2-(2,4,6-trimethyl-phenyl)-cyclopentane-1,3-dione

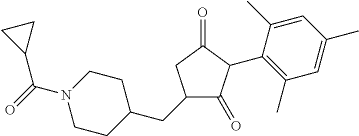 [SMILES: Cc1cc(C)c(C2C(=O)CC(CC3CCN(C(=O)C4CC4)CC3)C2=O)c(C)c1]

To a suspension of 4-(1-cyclopropanecarbonyl-piperidin-4-ylmethyl)-3-oxo-2-(2,4,6-trimethyl-phenyl)-cyclopent-1-enyl ester (162 mg, 0.36 mmol) in methanol is added potassium carbonate (149 mg, 1.08 mmol) and the reaction stirred at room temperature for 4 hours. The solvent is removed under reduced pressure and the residue dissolved in water (2 ml). 2N HCl (3 ml) is then added, and the resulting precipitate is filtered off, washed with hexane and air dried to give 4-(1-cyclopropanecarbonyl-piperidin-4-ylmethyl)-2-(2,4,6-trimethyl-phenyl)-cyclopentane-1,3-dione (72 mg).

### Example 7

### Preparation of the sodium salt of the 4-(tetrahydro-pyran-4-ylmethyl)-2-(2,4,6-trimethyl-phenyl)-cyclopentane-1,3-dione

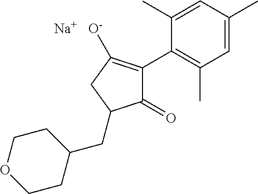 [SMILES: Cc1cc(C)c(C2=C([O-])CC(CC3CCOCC3)C2=O)c(C)c1 ; [Na+]]

A 0.5 M solution of sodium methoxide in methanol (2 ml, 1 mmol) is added to the 4-(tetrahydro-pyran-4-ylmethyl)-2-(2,4,6-trimethyl-phenyl)-cyclopentane-1,3-dione (314 mg, 1 mmol) at room temperature under nitrogen. The reaction mixture was stirred at room temperature for 15 minutes. The reaction mixture was evaporated under reduced pressure to give the sodium salt of the 4-(tetrahydro-pyran-4-ylmethyl)-2-(2,4,6-trimethyl-phenyl)-cyclopentane-1,3-dione (336 mg).

Where more than one tautomer or rotational conformer is observed in the proton NMR spectrum, the data shown below are for the mixture of isomers and conformers.

Unless otherwise stated, proton NMR spectra were recorded at ambient temperature.

Compounds characterised by HPLC-MS were analysed using one of three methods described below.

### Method A

Compounds characterised by HPLC-MS were analysed using a Waters 2795 HPLC equipped with a Waters Atlantis dC18 column (column length 20 mm, internal diameter of column 3 mm, particle size 3 micron, temperature 40° C.), Waters photodiode array and Micromass ZQ2000. The analysis was conducted using a three minutes run time, according to the following gradient table:

| Time | Solvent A | Solvent B | Flow (ml/ |
|---|---|---|---|
| (mins) | (%) | (%) | mn) |
| 0.00 | 90.0 | 10.0 | 2.00 |
| 0.25 | 90.0 | 10.0 | 2.00 |
| 2.00 | 10.0 | 90.0 | 2.00 |
| 2.50 | 10.0 | 90.0 | 2.00 |
| 2.60 | 90.0 | 10.0 | 2.00 |
| 3.0 | 90.0 | 10.0 | 2.00 |

Solvent A: H₂O containing 0.1% HCOOH
Solvent B: CH₃CN containing 0.1% HCOOH

### Method B

Compounds characterised by HPLC-MS were analysed using an Waters 2777 injector with a 1525 micro pump HPLC equipped with a Waters Atlantis dC18 IS column (column length 20 mm, internal diameter of column 3 mm, particle size 3 micron), Waters 2996 photodiode array, Waters 2420 ELSD and Micromass ZQ2000. The analysis was conducted using a three minutes run time, according to the following gradient table:

| Time | Solvent A | Solvent B | Flow (ml/ |
|---|---|---|---|
| (mins) | (%) | (%) | mn) |
| 0.00 | 95.0 | 5 | 1.300 |
| 2.50 | 0.00 | 100 | 1.300 |
| 2.80 | 0.00 | 100 | 1.300 |
| 2.90 | 95.0 | 5 | 1.300 |

Solvent A: H₂O with 0.05% TFA
Solvent B: CH₃CN with 0.05% TFA

### Method C:

Compounds characterised by HPLC-MS were analysed using a Finnigan Surveyor MSQ Plus equipped with a Waters Xterra column (column length 50 mm, internal diameter of column 4.6 mm, particle size 3.5 micron, temperature 40° C.), Waters photodiode array and Micromass ZQ2000. The analysis was conducted using a six minutes run time, according to the following gradient table:

| Time | Solvent A | Solvent B | Flow (ml/ |
|---|---|---|---|
| (mins) | (%) | (%) | mn) |
| 0.00 | 90.0 | 10.0 | 1.30 |
| 3.80 | 0.00 | 100 | 1.30 |
| 4.80 | 0.00 | 100 | 1.30 |
| 5.00 | 90.0 | 10.0 | 1.30 |
| 6.00 | 90.0 | 10.0 | 1.30 |

Solvent A: H₂O containing 0.05% HCOOH
Solvent B: CH₃CN containing 0.05% HCOOH

TABLE T1

| | | ¹H nmr (CDCl₃ |
|---|---|---|
| | | unless stated), |
| Compound | | LC/MS or other |
| Number | Structure | physical data |
| T1 | 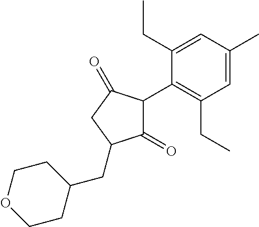 [SMILES: CCc1cc(C)cc(CC)c1C1C(=O)CC(CC2CCOCC2)C1=O] | □□ ppm 1.05-1.10 (m, 6H), 1.29-1.45 (m, 3H), 1.49-1.57 (m, 1H), 1.62-1.72 (m, 3H), 1.90-1.96 (m, 2H), 2.32-2.39 (m, 8H), 3.36-3.43 (m, 2H), 3.93-3.99 (m, 2H) |
| T2 | 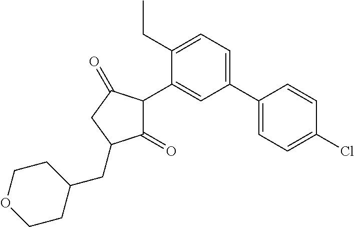 [SMILES: CCc1ccc(-c2ccc(Cl)cc2)cc1C1C(=O)CC(CC2CCOCC2)C1=O] | □ ppm 1.14 (t, 3H), 1.28-1.43 (m, 3H), 1.58-1.74 (m, 3H), 1.85-1.97 (m, 1H), 2.50 (q, 2H), 2.61-3.10 (m, 3H), 3.30-3.47 (m, 2H), 3.89-4.02 (m, 2H), 7.24 (d, 1H), 7.38-7.41 (m, 2H), 7.42 (d, 1H), 7.45-7.49 (m, 2H). 7.55 (dd, 1H) |
| T3 | 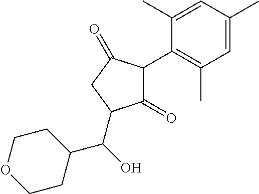 [SMILES: Cc1cc(C)c(C2C(=O)CC(C(O)C3CCOCC3)C2=O)c(C)c1] | □ ppm 1.50-1.58 (m, 2H), 1.66-1.82 (m, 3H), 2.09 (s, 6H), 2.27 (s, 3H), 2.32-2.43 (m, 1H), 2.69-2.82 (m, 1H), 2.96 (s, 1H), 3.35-3.48 (m, 2H), 3.67-3.76 (m, 1H), 3.99-4.11 (m, 2H), 6.90 (s, 2H) |
| T4 | 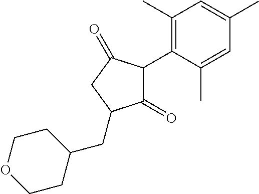 [SMILES: Cc1cc(C)c(C2C(=O)CC(CC3CCOCC3)C2=O)c(C)c1] | □ ppm 1.29-1.46 (m, 3H), 1.61-1.76 (m, 3H), 1.86-1.98 (m, 1H), 2.08 (d, 6H), 2.28 (s, 3H), 2.37 (d (br), 1H), 2.80-2.95 (m, 2H), 3.35-3.47 (m, 2H), 3.92-4.04 (m, 2H), 6.92 (s, 2H) |
| T5 | 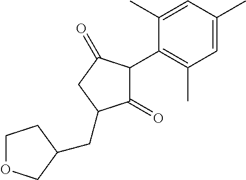 [SMILES: Cc1cc(C)c(C2C(=O)CC(CC3CCOC3)C2=O)c(C)c1] | □ ppm 1.48-1.68 (m, 2H), 2.05-2.13 (m, 8H), 2.28 (s, 3H), 2.33-2.46 (m, 2H), 2.80-2.98 (m (br), 2H), 3.36-3.47 (m, 1H), 3.78 (dd, 1H), 3.86-3.92 (m, 1H), 3.95 (dd, 1H), 6.92 (s, 2H) |
| T6 | 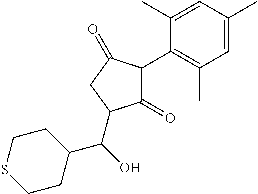 [SMILES: Cc1cc(C)c(C2C(=O)CC(C(O)C3CCSCC3)C2=O)c(C)c1] | □ ppm 1.46-1.61 (m, 2H), 1.66-1.86 (m, 1H), 1.93-2.05 (m, 2H), 2.07-2.09 (m, 6H), 2.27 (s, 3H), 2.65-2.83 (m, 6H), 2.91-3.05 (m, 1H), 3.67 (d, 1H), 6.90 (s, 2H) |
| T7 | 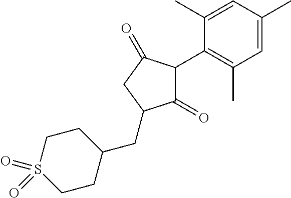 [SMILES: Cc1cc(C)c(C2C(=O)CC(CC3CCS(=O)(=O)CC3)C2=O)c(C)c1] | □ ppm 1.41-1.53 (m, 1H), 1.85-2.02 (m, 4H), 2.05-2.10 (m, 6H), 2.12-2.25 (m, 2H), 2.28 (s, 3H), 2.30-2.38 (m, 1H), 2.83-3.14 (m, 6H), 6.93 (s, 2H) |
| T8 | 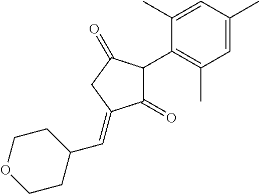 [SMILES: Cc1cc(C)c(C2C(=O)C/C(=C\C3CCOCC3)C2=O)c(C)c1] | □ ppm 1.51-1.73 (m, 4H), 2.10 (s, 6H), 2.29 (s, 3H), 2.40-2.55 (m, 1H), 3.13 (s, 2H), 3.48 (td, 2H), 3.92-4.06 (m, 2H), 6.01 (s, 1H), 6.94 (s, 2H) |
| T9 | 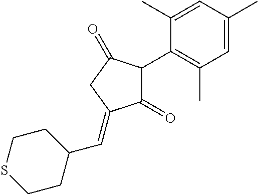 [SMILES: Cc1cc(C)c(C2C(=O)C/C(=C\C3CCSCC3)C2=O)c(C)c1] | □ ppm 1.67 (m, 2H), 1.98-2.08 (m, 2H), 2.10 (s, 6H), 2.27-2.30 (m, 4H), 2.63-2.83 (m, 4H), 3.09 (s, 2H), 6.00 (d, 1H), 6.94 (s, 2H) |
| T10 | 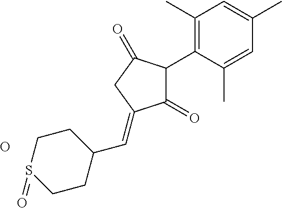 | □ ppm 2.10 (s, 6H), 2.13-2.25 (m, 5H), 2.29 (s, 3H), 2.44-2.62 (m, 1H), 2.99-3.27 (m, 6H), 6.02 (d, 1H), 6.94 (s, 2H) |
| T11 | 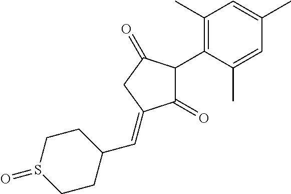 [SMILES: Cc1cc(C)c(C2C(=O)C/C(=C\C3CCS(=O)CC3)C2=O)c(C)c1] | LC-MS (Method A) ES⁺: MH⁺ = 345 rt = 1.17 min |
| T12 | 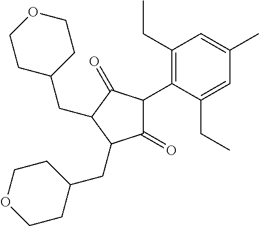 [SMILES: CCc1cc(C)cc(CC)c1C1C(=O)C(CC2CCOCC2)C(CC2CCOCC2)C1=O] | LC-MS (Method A) ES⁺: MH⁺ = 441 |
| T13 | 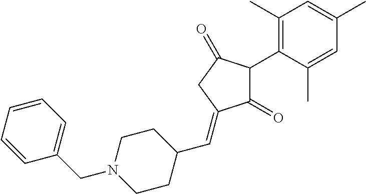 [SMILES: Cc1cc(C)c(C2C(=O)C/C(=C\C3CCN(Cc4ccccc4)CC3)C2=O)c(C)c1] | LC-MS (Method A) ES⁺: MH⁺ = 402 rt = 1.31 min |
| T14 | 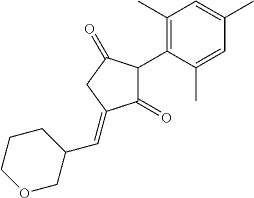 [SMILES: Cc1cc(C)c(C2C(=O)C/C(=C\C3CCCOC3)C2=O)c(C)c1] | LC-MS (Method A) ES⁺: MH⁺ = 313 rt = 1.39 min |
| T15 | 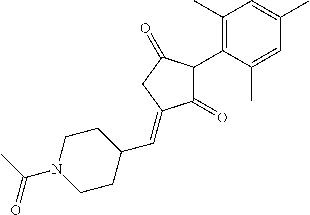 [SMILES: CC(=O)N1CCC(/C=C2\CC(=O)C(c3c(C)cc(C)cc3C)C2=O)CC1] | LC-MS (Method A) ES⁺: MH⁺ = 354 rt = 1.27 min |
| T16 | 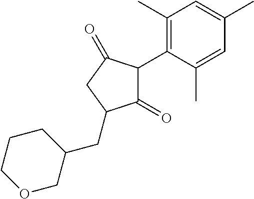 [SMILES: Cc1cc(C)c(C2C(=O)CC(CC3CCCOC3)C2=O)c(C)c1] | LC-MS (Method A) ES⁺: MH⁺ = 315 rt = 1.34 min |
| T17 | 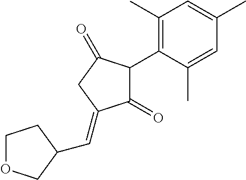 [SMILES: Cc1cc(C)c(C2C(=O)C/C(=C\C3CCOC3)C2=O)c(C)c1] | LC-MS (Method A) ES⁺: MH⁺ = 299 rt = 1.27 min |
| T18 | 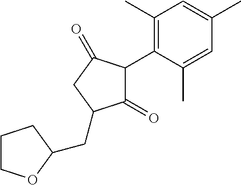 [SMILES: Cc1cc(C)c(C2C(=O)CC(CC3CCCO3)C2=O)c(C)c1] | LC-MS (Method A) ES⁺: MH⁺ = 301 rt = 1.36 min |
| T19 | 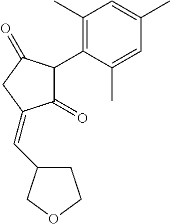 [SMILES: Cc1cc(C)c(C2C(=O)C/C(=C/C3CCOC3)C2=O)c(C)c1] | LC-MS (Method A) ES⁺: MH⁺ = 299 rt = 1.32 min |
| T20 | 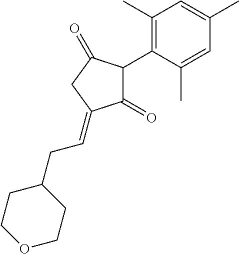 [SMILES: Cc1cc(C)c(C2C(=O)C/C(=C\CC3CCOCC3)C2=O)c(C)c1] | □ ppm 1.19-1.38 (m, 2H), 1.52-1.72 (m, 3H), 2.05 (s, 6H), 2.03-2.07 (m, 2H), 2.25 (s, 3H), 2.94 (s, 2H), 3.24-3.30 (m, 2H), 3.79-2.83 (m, 2H), 6.21 (t, 1H), 6.87 (s, 2H) |
| T21 | 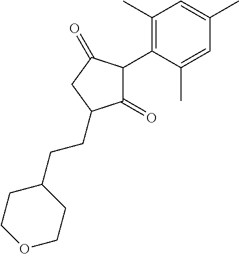 [SMILES: Cc1cc(C)c(C2C(=O)CC(CCC3CCOCC3)C2=O)c(C)c1] | LC-MS (Method B) ES⁺: MH⁺ = 329 rt = 1.34 min |
| T22 | 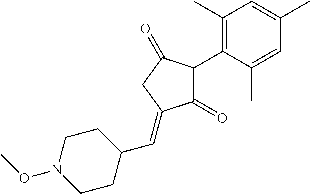 | LC-MS (Method A) ES⁺: MH⁺ = 344 rt = 1.27 min |
| T23 | 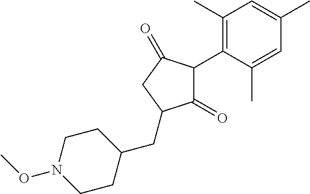 | LC-MS (Method A) ES⁺: MH⁺ = 344 rt = 1.19 min |
| T24 | 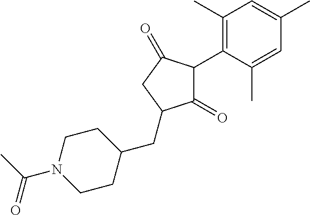 [SMILES: CC(=O)N1CCC(CC2CC(=O)C(c3c(C)cc(C)cc3C)C2=O)CC1] | LC-MS (Method A) ES⁺: MH⁺ = 356 rt = 1.29 min |
| T25 | 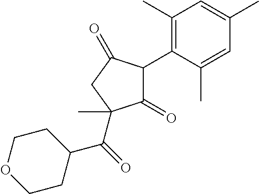 [SMILES: Cc1cc(C)c(C2C(=O)CC(C)(C(=O)C3CCOCC3)C2=O)c(C)c1] | LC-MS (Method B) ES⁺: MH⁺ = 343 rt = 1.33 min |
| T26 | 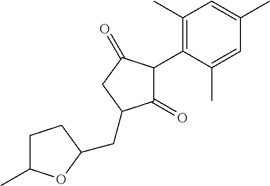 | LC-MS (Method A) ES⁺: MH⁺ = 315 rt = 1.46 min |
| T27 | 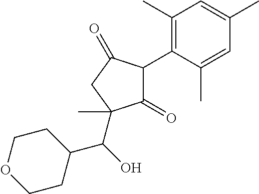 [SMILES: Cc1cc(C)c(C2C(=O)CC(C)(C(O)C3CCOCC3)C2=O)c(C)c1] | LC-MS (Method B) ES⁺: MH⁺ = 345 rt = 1.10 min |
| T28 | 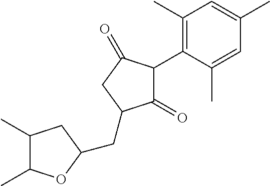 | LC-MS (Method A) ES⁺: MH⁺ = 329 rt = 1.52 min |
| T29 | 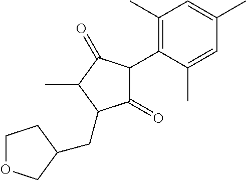 [SMILES: Cc1cc(C)c(C2C(=O)C(C)C(CC3CCOC3)C2=O)c(C)c1] | LC-MS (Method B) ES⁺: MH⁺ = 315 rt = 1.24 min |
| T30 | 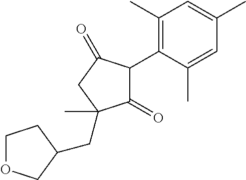 [SMILES: Cc1cc(C)c(C2C(=O)CC(C)(CC3CCOC3)C2=O)c(C)c1] | LC-MS (Method B) ES⁺: MH⁺ = 315 rt = 1.21 min |
| T31 | 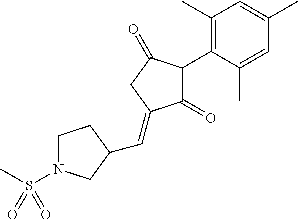 | LC-MS (Method A) ES⁺: MH⁺ = 376 rt = 1.29 min |
| T32 | 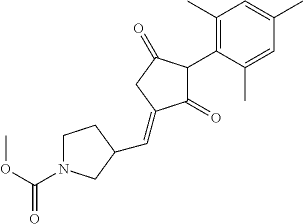 [SMILES: COC(=O)N1CCC(/C=C2\CC(=O)C(c3c(C)cc(C)cc3C)C2=O)C1] | LC-MS (Method A) ES⁺: MH⁺ = 356 rt = 1.36 min |
| T33 | 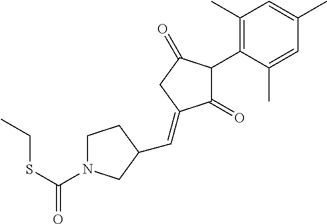 [SMILES: CCSC(=O)N1CCC(/C=C2\CC(=O)C(c3c(C)cc(C)cc3C)C2=O)C1] | LC-MS (Method A) ES⁺: MH⁺ = 386 rt = 1.51 min |
| T34 | 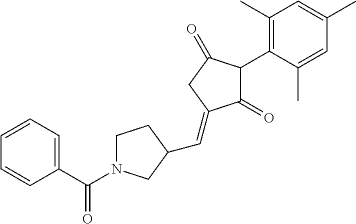 [SMILES: Cc1cc(C)c(C2C(=O)C/C(=C\C3CCN(C(=O)c4ccccc4)C3)C2=O)c(C)c1] | LC-MS (Method A) ES⁺: MH⁺ = 402 rt = 1.42 min |
| T35 | 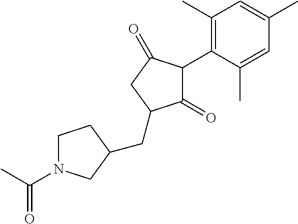 [SMILES: CC(=O)N1CCC(CC2CC(=O)C(c3c(C)cc(C)cc3C)C2=O)C1] | LC-MS (Method A) ES⁺: MH⁺ = 342 rt = 1.21 min |
| T36 | 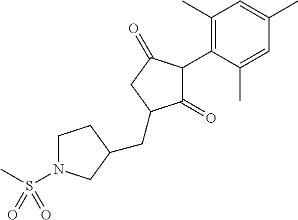 | LC-MS (Method A) ES⁺: MH⁺ = 378 rt = 1.29 min |
| T37 | 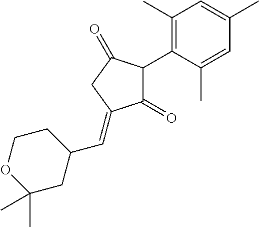 | LC-MS (Method A) ES⁺: MH⁺ = 341 rt = 1.44 min |
| T38 | 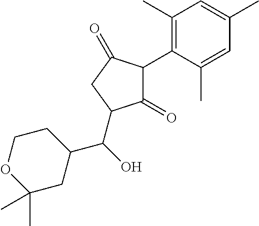 | LC-MS (Method A) ES⁺: MH⁺ = 359 rt = 1.22 min |
| T39 | 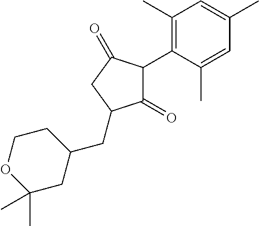 | LC-MS (Method A) ES⁺: MH⁺ = 343 rt = 1.42 min |
| T40 | 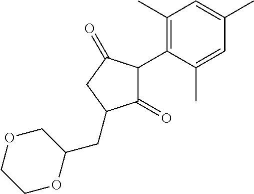 [SMILES: Cc1cc(C)c(C2C(=O)CC(CC3COCCO3)C2=O)c(C)c1] | LC-MS (Method A) ES⁺: MH⁺ = 317 rt = 1.22 min |
| T41 | 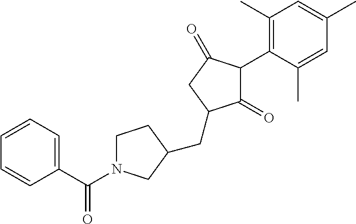 [SMILES: Cc1cc(C)c(C2C(=O)CC(CC3CCN(C(=O)c4ccccc4)C3)C2=O)c(C)c1] | LC-MS (Method A) ES⁺: MH⁺ = 404 rt = 1.41 min |
| T42 | 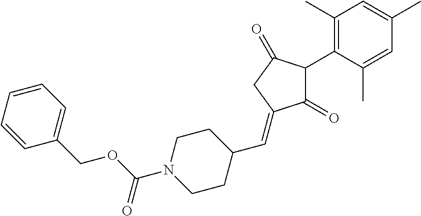 [SMILES: Cc1cc(C)c(C2C(=O)C/C(=C\C3CCN(C(=O)OCc4ccccc4)CC3)C2=O)c(C)c1] | LC-MS (Method A) ES⁺: MH⁺ = 446 rt = 1.66 min |
| T43 | 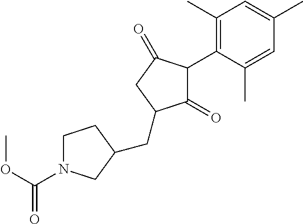 [SMILES: COC(=O)N1CCC(CC2CC(=O)C(c3c(C)cc(C)cc3C)C2=O)C1] | LC-MS (Method A) ES⁺: MH⁺ = 358 rt = 1.34 min |
| T44 | 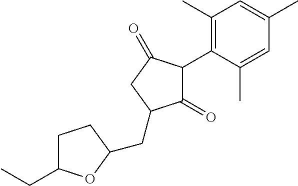 [SMILES: CCC1CCC(CC2CC(=O)C(c3c(C)cc(C)cc3C)C2=O)O1] | LC-MS (Method A) ES⁺: MH⁺ = 329 rt = 1.59 min |
| T45 | 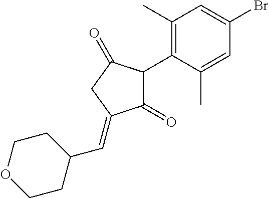 [SMILES: Cc1cc(Br)cc(C)c1C1C(=O)C/C(=C\C2CCOCC2)C1=O] | □□□ DMSO-d6 □□ ppm 1.45 (m, 2H), 1.59 (m, 2H), 2.01 (s, 6H), 3.08 (br, s), 3.37 (m, 2H), 3.87 (m, 2H), 5.75 (s, 1H), 6.04 (br s, 1H), 7.27 (s, 2H). |
| T46 | 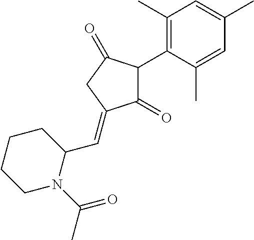 [SMILES: CC(=O)N1CCCCC1/C=C1\CC(=O)C(c2c(C)cc(C)cc2C)C1=O] | LC-MS (Method A) ES⁺: MH⁺ = 354 rt = 1.32 min |
| T47 | 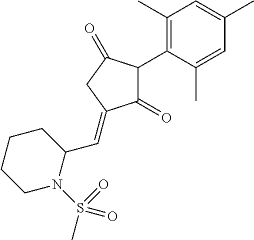 [SMILES: Cc1cc(C)c(C2C(=O)C/C(=C\C3CCCCN3S(C)(=O)=O)C2=O)c(C)c1] | LC-MS (Method A) ES⁺: MH⁺ = 390 rt = 1.38 min |
| T48 | 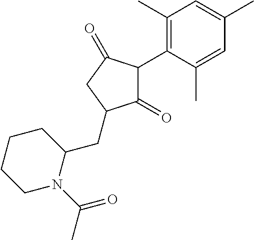 [SMILES: CC(=O)N1CCCCC1CC1CC(=O)C(c2c(C)cc(C)cc2C)C1=O] | LC-MS (Method A) ES⁺: MH⁺ = 356 rt = 1.38 min |
| T49 | 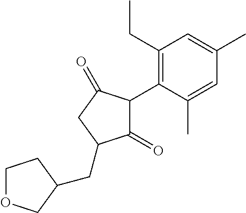 [SMILES: CCc1cc(C)cc(C)c1C1C(=O)CC(CC2CCOC2)C1=O] | LC-MS (Method B) ES⁺: MH⁺ = 315 rt = 1.34 min |
| T50 | 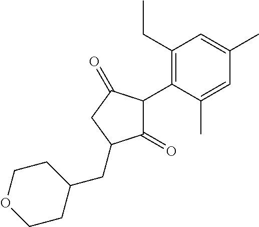 [SMILES: CCc1cc(C)cc(C)c1C1C(=O)CC(CC2CCOCC2)C1=O] | LC-MS (Method B) ES⁺: MH⁺ = 329 rt = 1.29 min |
| T51 | 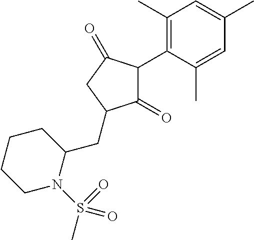 [SMILES: Cc1cc(C)c(C2C(=O)CC(CC3CCCCN3S(C)(=O)=O)C2=O)c(C)c1] | LC-MS (Method A) ES⁺: MH⁺ = 392 rt = 1.36 min |
| T52 | 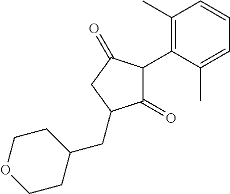 [SMILES: Cc1cccc(C)c1C1C(=O)CC(CC2CCOCC2)C1=O] | LC-MS (Method C) ES−: M − H⁺ = 299 Rt = 4.75 mins Melting point: 165-167° C. |
| T53 | 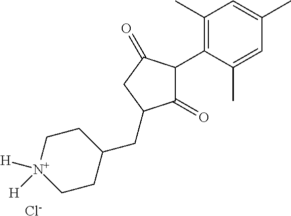 [SMILES: Cc1cc(C)c(C2C(=O)CC(CC3CC[NH2+]CC3)C2=O)c(C)c1 ; [Cl-]] | LC-MS (Method A) ES⁺: MH⁺ = 314 rt = 1.09 min |
| T54 | 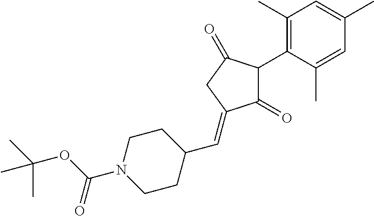 [SMILES: Cc1cc(C)c(C2C(=O)C/C(=C\C3CCN(C(=O)OC(C)(C)C)CC3)C2=O)c(C)c1] | LC-MS (Method A) ES⁺: MH⁺ = 412 rt = 1.63 min |
| T55 | 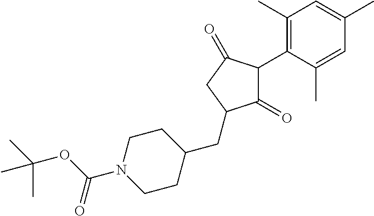 [SMILES: Cc1cc(C)c(C2C(=O)CC(CC3CCN(C(=O)OC(C)(C)C)CC3)C2=O)c(C)c1] | LC-MS (Method A) ES⁺: MH⁺ = 414 rt = 1.61 min |
| T56 | 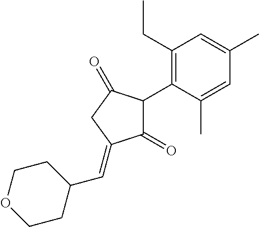 [SMILES: CCc1cc(C)cc(C)c1C1C(=O)C/C(=C\C2CCOCC2)C1=O] | LC-MS (Method B) ES⁺: MH⁺ = 327 rt = 1.39 min |
| T57 | 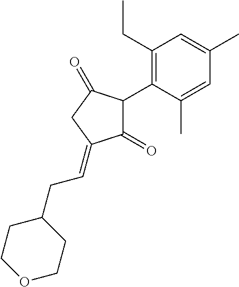 [SMILES: CCc1cc(C)cc(C)c1C1C(=O)C/C(=C\CC2CCOCC2)C1=O] | LC-MS (Method B) ES⁺: MH⁺ = 341 rt = 1.39 min |
| T58 | 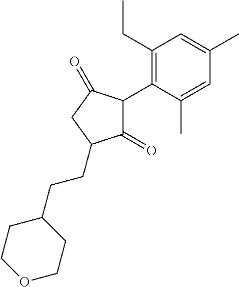 [SMILES: CCc1cc(C)cc(C)c1C1C(=O)CC(CCC2CCOCC2)C1=O] | LC-MS (Method B) ES⁺: MH⁺ = 343 rt = 1.34 min |
| T59 | 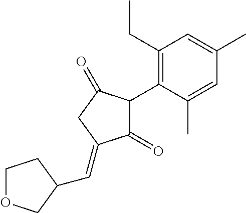 [SMILES: CCc1cc(C)cc(C)c1C1C(=O)C/C(=C\C2CCOC2)C1=O] | LC-MS (Method B) ES⁺: MH⁺ = 313 rt = 1.29 min |
| T60 | 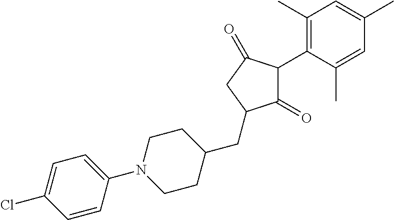 [SMILES: Cc1cc(C)c(C2C(=O)CC(CC3CCN(c4ccc(Cl)cc4)CC3)C2=O)c(C)c1] | LC-MS (Method A) ES⁺: MH⁺ = 426, 424 rt = 1.56 min |
| T61 | 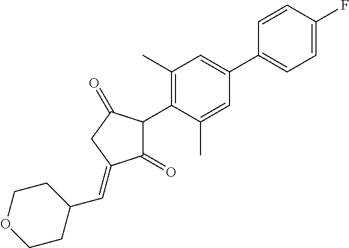 [SMILES: Cc1cc(-c2ccc(F)cc2)cc(C)c1C1C(=O)C/C(=C\C2CCOCC2)C1=O] | Melting point: 230-232° C. |
| T62 | 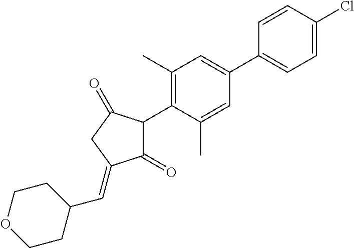 [SMILES: Cc1cc(-c2ccc(Cl)cc2)cc(C)c1C1C(=O)C/C(=C\C2CCOCC2)C1=O] | LC-MS (Method C) ES−: M − H⁺ = 407, 409 rt = 5.77 min Melting point: 242-244° C. |
| T63 | 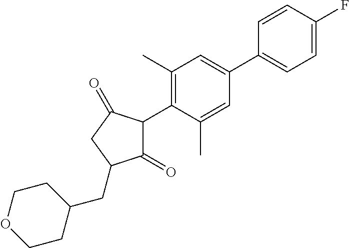 [SMILES: Cc1cc(-c2ccc(F)cc2)cc(C)c1C1C(=O)CC(CC2CCOCC2)C1=O] | Melting point: 115-117° C. |
| T64 | 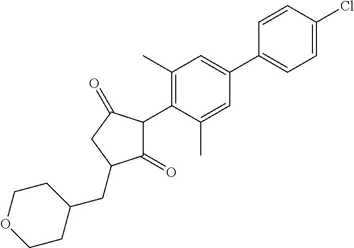 [SMILES: Cc1cc(-c2ccc(Cl)cc2)cc(C)c1C1C(=O)CC(CC2CCOCC2)C1=O] | Melting point: 232-233° C. |
| T65 | 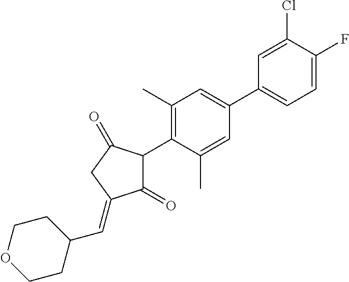 [SMILES: Cc1cc(-c2ccc(F)c(Cl)c2)cc(C)c1C1C(=O)C/C(=C\C2CCOCC2)C1=O] | Melting point: 225-227° C. |
| T66 | 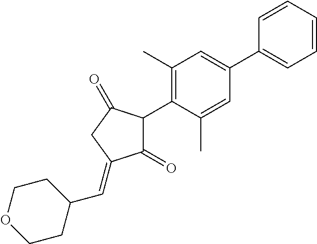 [SMILES: Cc1cc(-c2ccccc2)cc(C)c1C1C(=O)C/C(=C\C2CCOCC2)C1=O] | LC-MS (Method C) ES⁺: MH⁺ = 375 rt = 4.93 min |
| T67 | 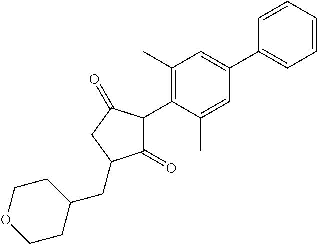 [SMILES: Cc1cc(-c2ccccc2)cc(C)c1C1C(=O)CC(CC2CCOCC2)C1=O] | LC-MS (Method C) ES⁺: MH⁺ = 377 rt = 4.05 min |
| T68 | 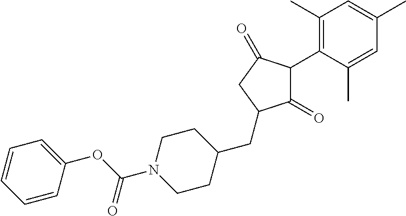 [SMILES: Cc1cc(C)c(C2C(=O)CC(CC3CCN(C(=O)Oc4ccccc4)CC3)C2=O)c(C)c1] | LC-MS (Method B) ES⁺: MH⁺ = 434 rt = 1.61 min |
| T69 | 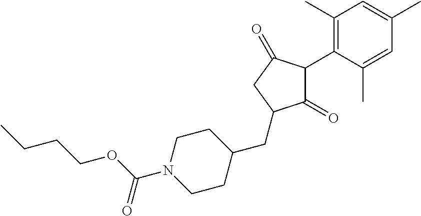 [SMILES: CCCCOC(=O)N1CCC(CC2CC(=O)C(c3c(C)cc(C)cc3C)C2=O)CC1] | LC-MS (Method B) ES⁺: MH⁺ = 414 rt = 1.64 min |
| T70 | 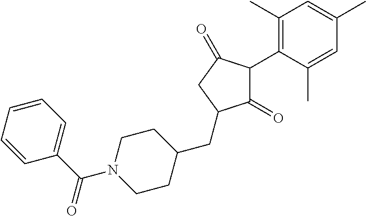 [SMILES: Cc1cc(C)c(C2C(=O)CC(CC3CCN(C(=O)c4ccccc4)CC3)C2=O)c(C)c1] | LC-MS (Method B) ES⁺: MH⁺ = 418 rt = 1.44 min |
| T71 | 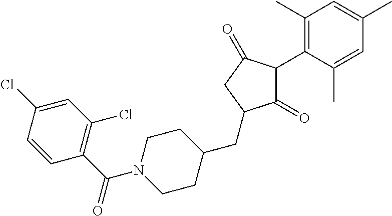 [SMILES: Cc1cc(C)c(C2C(=O)CC(CC3CCN(C(=O)c4ccc(Cl)cc4Cl)CC3)C2=O)c(C)c1] | LC-MS (Method B) ES⁺: MH⁺ = 490, 488, 486 rt = 1.61 min |
| T72 | 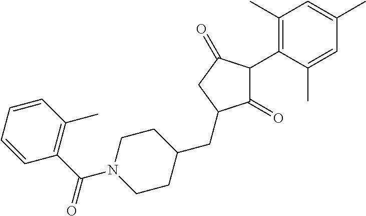 [SMILES: Cc1cc(C)c(C2C(=O)CC(CC3CCN(C(=O)c4ccccc4C)CC3)C2=O)c(C)c1] | LC-MS (Method B) ES⁺: MH⁺ = 432 rt = 1.49 min |
| T73 | 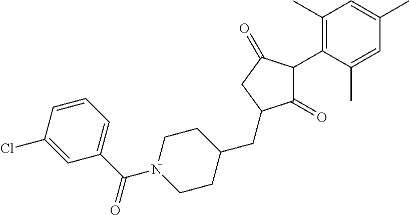 [SMILES: Cc1cc(C)c(C2C(=O)CC(CC3CCN(C(=O)c4cccc(Cl)c4)CC3)C2=O)c(C)c1] | LC-MS (Method B) ES⁺: MH⁺ = 454, 452 rt = 1.55 min |
| T74 | 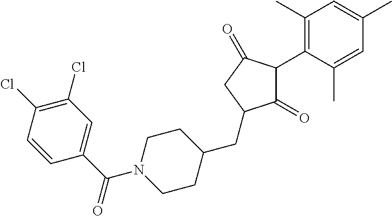 [SMILES: Cc1cc(C)c(C2C(=O)CC(CC3CCN(C(=O)c4ccc(Cl)c(Cl)c4)CC3)C2=O)c(C)c1] | LC-MS (Method B) ES⁺: MH⁺ = 490, 488, 486 rt = 1.64 min |
| T75 | 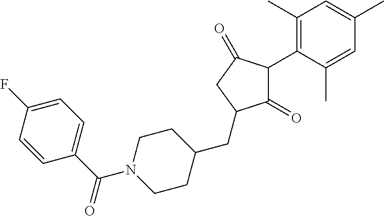 [SMILES: Cc1cc(C)c(C2C(=O)CC(CC3CCN(C(=O)c4ccc(F)cc4)CC3)C2=O)c(C)c1] | LC-MS (Method B) ES⁺: MH⁺ = 436 rt = 1.48 min |
| T76 | 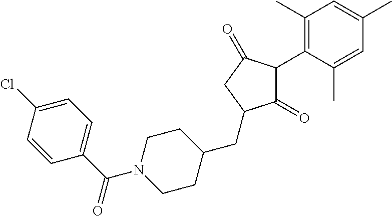 [SMILES: Cc1cc(C)c(C2C(=O)CC(CC3CCN(C(=O)c4ccc(Cl)cc4)CC3)C2=O)c(C)c1] | LC-MS (Method B) ES⁺: MH⁺ = 454, 452 rt = 1.56 min |
| T77 | 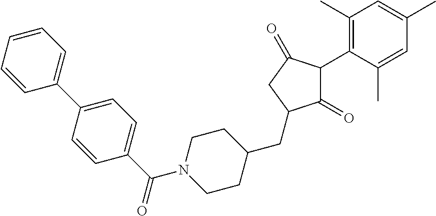 [SMILES: Cc1cc(C)c(C2C(=O)CC(CC3CCN(C(=O)c4ccc(-c5ccccc5)cc4)CC3)C2=O)c(C)c1] | LC-MS (Method B) ES⁺: MH⁺ = 494 rt = 1.70 min |
| T78 | 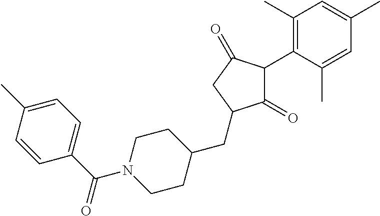 | LC-MS (Method B) ES⁺: MH⁺ = 432 rt = 1.52 min |
| T79 | 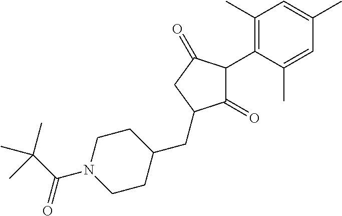 [SMILES: Cc1cc(C)c(C2C(=O)CC(CC3CCN(C(=O)C(C)(C)C)CC3)C2=O)c(C)c1] | LC-MS (Method B) ES⁺: MH⁺ = 398 rt = 1.47 min |
| T80 | 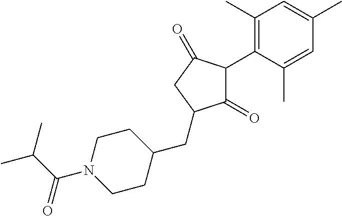 [SMILES: Cc1cc(C)c(C2C(=O)CC(CC3CCN(C(=O)C(C)C)CC3)C2=O)c(C)c1] | LC-MS (Method B) ES⁺: MH⁺ = 384 rt = 1.38 min |
| T81 | 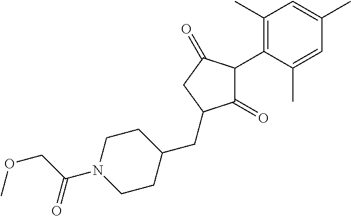 | LC-MS (Method B) ES⁺: MH⁺ = 386 rt = 1.25 min |
| T82 | 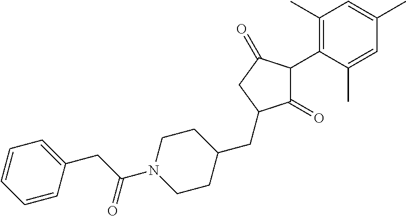 [SMILES: Cc1cc(C)c(C2C(=O)CC(CC3CCN(C(=O)Cc4ccccc4)CC3)C2=O)c(C)c1] | LC-MS (Method B) ES⁺: MH⁺ = 432 rt = 1.49 min |
| T83 | 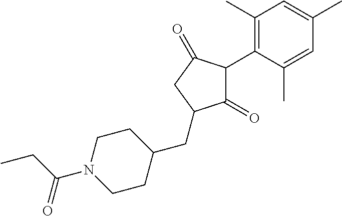 [SMILES: CCC(=O)N1CCC(CC2CC(=O)C(c3c(C)cc(C)cc3C)C2=O)CC1] | LC-MS (Method B) ES⁺: MH⁺ = 370 rt = 1.32 min |
| T84 | 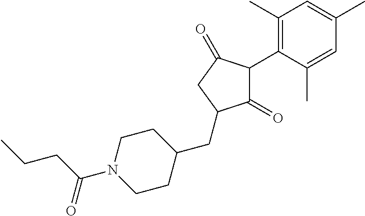 [SMILES: CCCC(=O)N1CCC(CC2CC(=O)C(c3c(C)cc(C)cc3C)C2=O)CC1] | LC-MS (Method B) ES⁺: MH⁺ = 384 rt = 1.39 min |
| T85 | 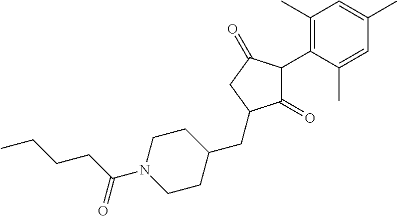 [SMILES: CCCCC(=O)N1CCC(CC2CC(=O)C(c3c(C)cc(C)cc3C)C2=O)CC1] | LC-MS (Method B) ES⁺: MH⁺ = 398 rt = 1.48 min |
| T86 | 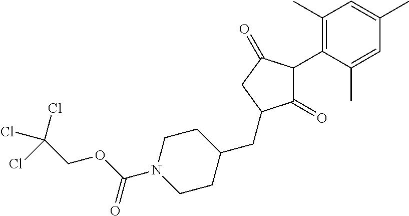 [SMILES: Cc1cc(C)c(C2C(=O)CC(CC3CCN(C(=O)OCC(Cl)(Cl)Cl)CC3)C2=O)c(C)c1] | LC-MS (Method B) ES⁺: MH⁺ = 492, 490, 488 rt = 1.71 min |
| T87 | 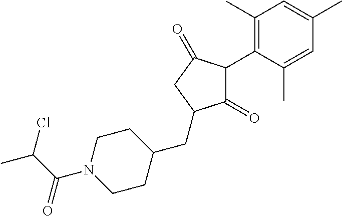 [SMILES: Cc1cc(C)c(C2C(=O)CC(CC3CCN(C(=O)C(C)Cl)CC3)C2=O)c(C)c1] | LC-MS (Method B) ES⁺: MH⁺ = 406, 404 rt = 1.42 min |
| T88 | 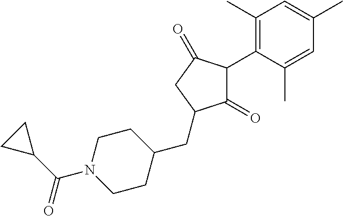 [SMILES: Cc1cc(C)c(C2C(=O)CC(CC3CCN(C(=O)C4CC4)CC3)C2=O)c(C)c1] | LC-MS (Method B) ES⁺: MH⁺ = 382 rt = 1.36 min |
| T89 | 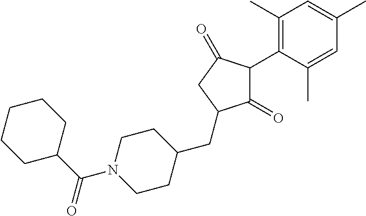 [SMILES: Cc1cc(C)c(C2C(=O)CC(CC3CCN(C(=O)C4CCCCC4)CC3)C2=O)c(C)c1] | LC-MS (Method B) ES⁺: MH⁺ = 424 rt = 1.55 min |
| T90 | 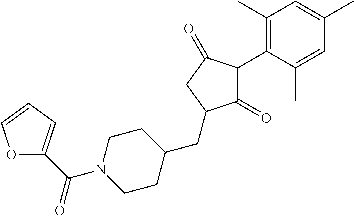 [SMILES: Cc1cc(C)c(C2C(=O)CC(CC3CCN(C(=O)c4ccco4)CC3)C2=O)c(C)c1] | LC-MS (Method B) ES⁺: MH⁺ = 408 rt = 1.38 min |
| T91 | 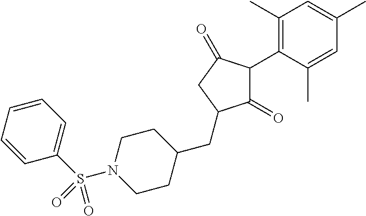 [SMILES: Cc1cc(C)c(C2C(=O)CC(CC3CCN(S(=O)(=O)c4ccccc4)CC3)C2=O)c(C)c1] | LC-MS (Method B) ES⁺: MH⁺ = 454 rt = 1.60 min |
| T92 | 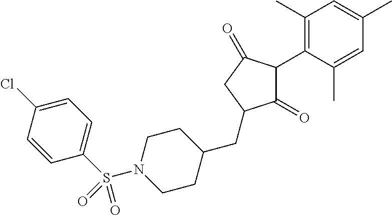 [SMILES: Cc1cc(C)c(C2C(=O)CC(CC3CCN(S(=O)(=O)c4ccc(Cl)cc4)CC3)C2=O)c(C)c1] | LC-MS (Method B) ES⁺: MH⁺ = 490, 488 rt = 1.70 min |
| T93 | 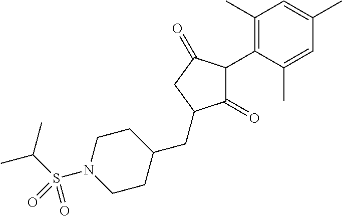 [SMILES: Cc1cc(C)c(C2C(=O)CC(CC3CCN(S(=O)(=O)C(C)C)CC3)C2=O)c(C)c1] | |
| T94 | 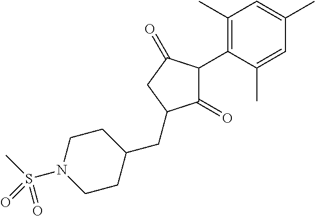 | LC-MS (Method B) ES⁺: MH⁺ = 392 rt = 1.34 min |
| T95 | 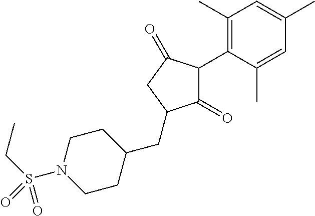 [SMILES: CCS(=O)(=O)N1CCC(CC2CC(=O)C(c3c(C)cc(C)cc3C)C2=O)CC1] | LC-MS (Method B) ES⁺: MH⁺ = 406 rt = 1.39 min |
| T96 | 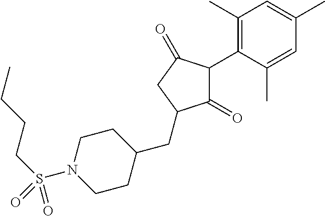 [SMILES: CCCCS(=O)(=O)N1CCC(CC2CC(=O)C(c3c(C)cc(C)cc3C)C2=O)CC1] | LC-MS (Method B) ES⁺: MH⁺ = 434 rt = 1.60 min |
| T97 | 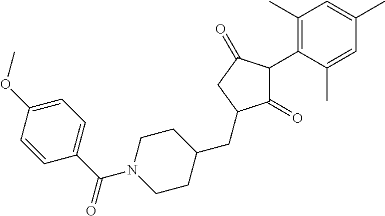 [SMILES: COc1ccc(C(=O)N2CCC(CC3CC(=O)C(c4c(C)cc(C)cc4C)C3=O)CC2)cc1] | LC-MS (Method B) ES⁺: MH⁺ = 448 rt = 1.47 min |
| T98 | 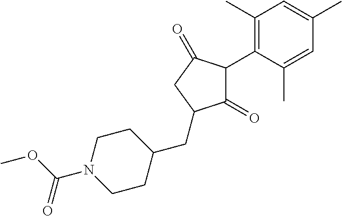 | LC-MS (Method B) ES⁺: MH⁺ = 372 rt = 1.40 min |
| T99 | 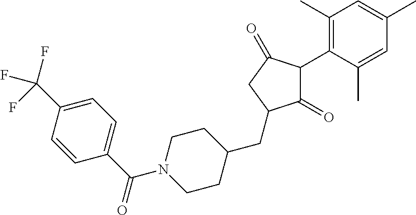 [SMILES: Cc1cc(C)c(C2C(=O)CC(CC3CCN(C(=O)c4ccc(C(F)(F)F)cc4)CC3)C2=O)c(C)c1] | LC-MS (Method B) ES⁺: MH⁺ = 486 rt = 1.60 min |
| T100 | 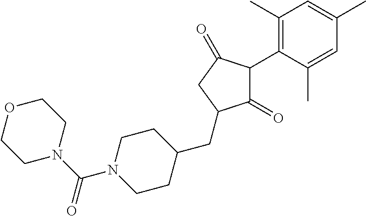 [SMILES: Cc1cc(C)c(C2C(=O)CC(CC3CCN(C(=O)N4CCOCC4)CC3)C2=O)c(C)c1] | LC-MS (Method B) ES⁺: MH⁺ = 427 rt = 1.31 min |
| T101 | 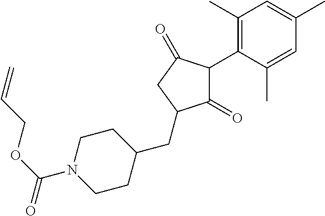 [SMILES: C=CCOC(=O)N1CCC(CC2CC(=O)C(c3c(C)cc(C)cc3C)C2=O)CC1] | LC-MS (Method B) ES⁺: MH⁺ = 398 rt = 1.51 min |
| T102 | 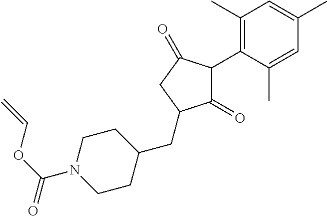 [SMILES: C=COC(=O)N1CCC(CC2CC(=O)C(c3c(C)cc(C)cc3C)C2=O)CC1] | LC-MS (Method B) ES⁺: MH⁺ = 384 rt = 1.51 min |
| T103 | 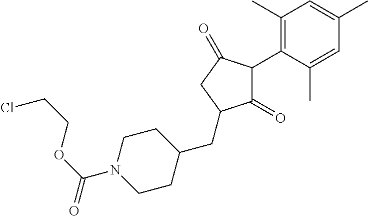 [SMILES: Cc1cc(C)c(C2C(=O)CC(CC3CCN(C(=O)OCCCl)CC3)C2=O)c(C)c1] | LC-MS (Method B) ES⁺: MH⁺ = 422, 420 rt = 1.52 min |
| T104 | 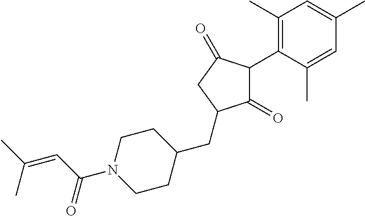 [SMILES: CC(C)=CC(=O)N1CCC(CC2CC(=O)C(c3c(C)cc(C)cc3C)C2=O)CC1] | LC-MS (Method B) ES⁺: MH⁺ = 396 rt = 1.41 min |
| T105 | 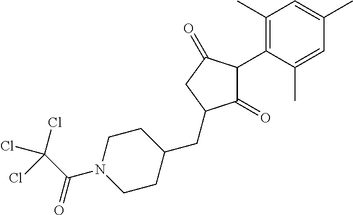 [SMILES: Cc1cc(C)c(C2C(=O)CC(CC3CCN(C(=O)C(Cl)(Cl)Cl)CC3)C2=O)c(C)c1] | LC-MS (Method B) ES⁺: MH⁺ = 462, 460, 458 rt = 1.64 min |
| T106 | 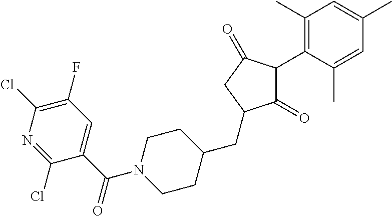 [SMILES: Cc1cc(C)c(C2C(=O)CC(CC3CCN(C(=O)c4cc(F)c(Cl)nc4Cl)CC3)C2=O)c(C)c1] | LC-MS (Method B) ES⁺: MH⁺ = 509, 507, 505 rt = 1.58 min |
| T107 | 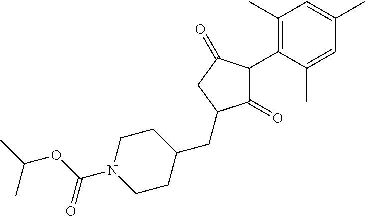 [SMILES: Cc1cc(C)c(C2C(=O)CC(CC3CCN(C(=O)OC(C)C)CC3)C2=O)c(C)c1] | LC-MS (Method B) ES⁺: MH⁺ = 400 rt = 1.54 min |
| T108 | 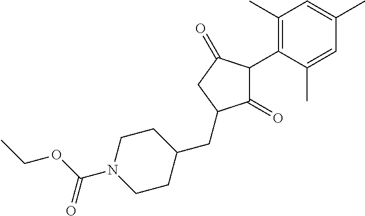 [SMILES: CCOC(=O)N1CCC(CC2CC(=O)C(c3c(C)cc(C)cc3C)C2=O)CC1] | LC-MS (Method B) ES⁺: MH⁺ = 386 rt = 1.46 min |
| T109 | 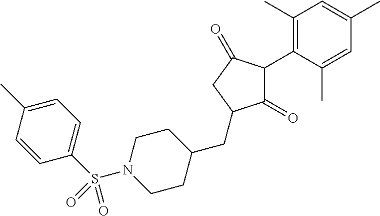 | LC-MS (Method B) ES⁺: MH⁺ = 468 rt = 1.65 min |
| T110 | 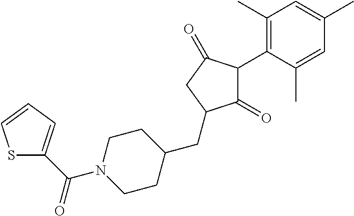 [SMILES: Cc1cc(C)c(C2C(=O)CC(CC3CCN(C(=O)c4cccs4)CC3)C2=O)c(C)c1] | LC-MS (Method B) ES⁺: MH⁺ = 424 rt = 1.44 min |
| T111 | 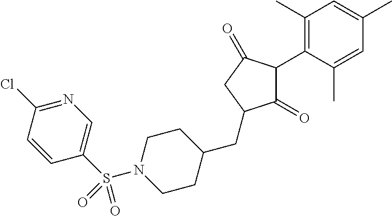 | LC-MS (Method B) ES⁺: MH⁺ = 455, 453 rt = 1.42 min |
| T112 | 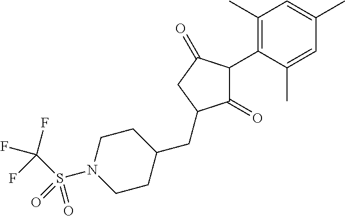 [SMILES: Cc1cc(C)c(C2C(=O)CC(CC3CCN(S(=O)(=O)C(F)(F)F)CC3)C2=O)c(C)c1] | LC-MS (Method B) ES⁺: MH⁺ = 446 rt = 1.66 min |
| T113 | 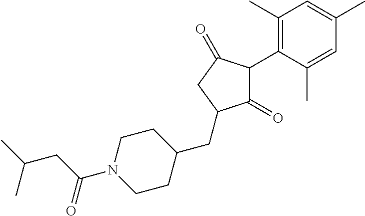 [SMILES: Cc1cc(C)c(C2C(=O)CC(CC3CCN(C(=O)CC(C)C)CC3)C2=O)c(C)c1] | LC-MS (Method B) ES⁺: MH⁺ = 398 rt = 1.44 min |
| T114 | 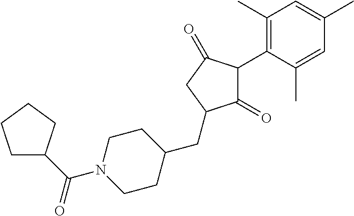 [SMILES: Cc1cc(C)c(C2C(=O)CC(CC3CCN(C(=O)C4CCCC4)CC3)C2=O)c(C)c1] | LC-MS (Method B) ES⁺: MH⁺ = 410 rt = 1.50 min |
| T115 | 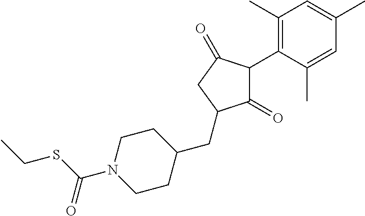 [SMILES: CCSC(=O)N1CCC(CC2CC(=O)C(c3c(C)cc(C)cc3C)C2=O)CC1] | LC-MS (Method B) ES⁺: MH⁺ = 402 rt = 1.56 min |
| T116 | 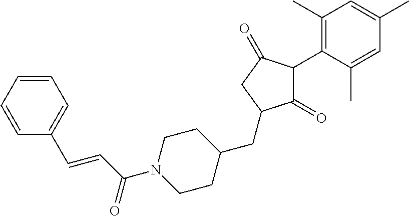 [SMILES: Cc1cc(C)c(C2C(=O)CC(CC3CCN(C(=O)/C=C/c4ccccc4)CC3)C2=O)c(C)c1] | LC-MS (Method B) ES⁺: MH⁺ = 444 rt = 1.55 min |
| T117 | 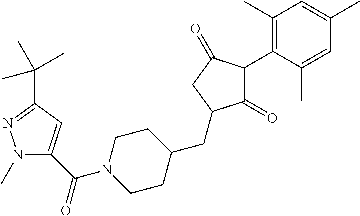 | LC-MS (Method B) ES⁺: MH⁺ = 478 rt = 1.54 min |
| T118 | 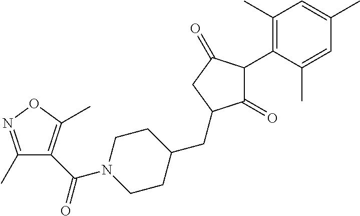 | LC-MS (Method B) ES⁺: MH⁺ = 437 rt = 1.35 min |
| T119 | 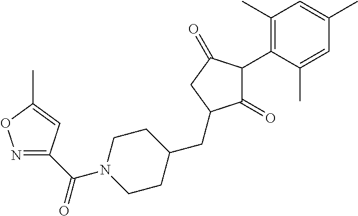 [SMILES: Cc1cc(C)c(C2C(=O)CC(CC3CCN(C(=O)c4cc(C)on4)CC3)C2=O)c(C)c1] | LC-MS (Method B) ES⁺: MH⁺ = 423 rt = 1.42 min |
| T120 | 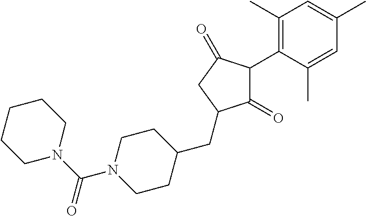 [SMILES: Cc1cc(C)c(C2C(=O)CC(CC3CCN(C(=O)N4CCCCC4)CC3)C2=O)c(C)c1] | LC-MS (Method B) ES⁺: MH⁺ = 425 rt = 1.51 min |
| T121 | 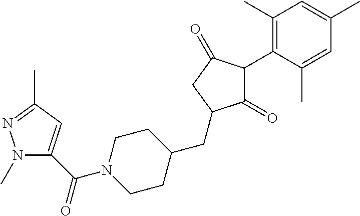 | LC-MS (Method B) ES⁺: MH⁺ = 436 rt = 1.36 min |
| T122 | 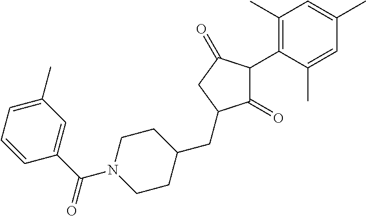 [SMILES: Cc1cccc(C(=O)N2CCC(CC3CC(=O)C(c4c(C)cc(C)cc4C)C3=O)CC2)c1] | LC-MS (Method B) ES⁺: MH⁺ = 432 rt = 1.52 min |
| T123 | 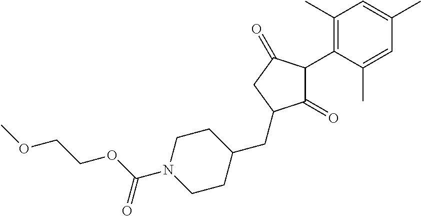 | LC-MS (Method B) ES⁺: MH⁺ = 416 rt = 1.38 min |
| T124 | 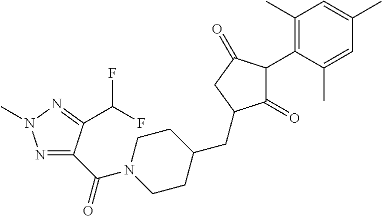 | LC-MS (Method B) ES⁺: MH⁺ = 472 rt = 1.35 min |
| T125 | 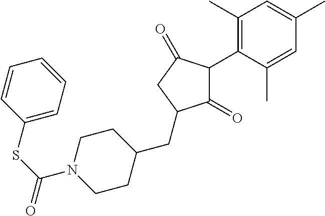 [SMILES: Cc1cc(C)c(C2C(=O)CC(CC3CCN(C(=O)Sc4ccccc4)CC3)C2=O)c(C)c1] | LC-MS (Method B) ES⁺: MH⁺ = 450 rt = 1.67 min |
| T126 | 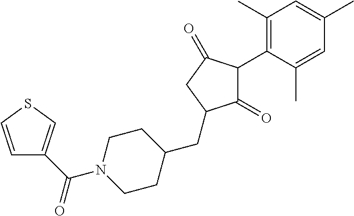 [SMILES: Cc1cc(C)c(C2C(=O)CC(CC3CCN(C(=O)c4ccsc4)CC3)C2=O)c(C)c1] | |
| T127 | 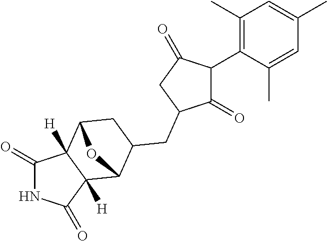 [SMILES: Cc1cc(C)c(C2C(=O)CC(CC3C[C@@H]4O[C@H]3[C@@H]3C(=O)NC(=O)[C@@H]34)C2=O)c(C)c1] | LC-MS (Method A) ES⁺: MH⁺ = 396 rt = 1.12 min |
| T128 | 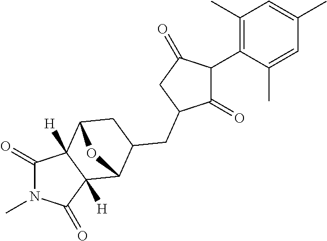 [SMILES: Cc1cc(C)c(C2C(=O)CC(CC3C[C@@H]4O[C@H]3[C@@H]3C(=O)N(C)C(=O)[C@@H]34)C2=O)c(C)c1] | LC-MS (Method A) ES⁺: MH⁺ = 410 rt = 1.21 min |
| T129 | 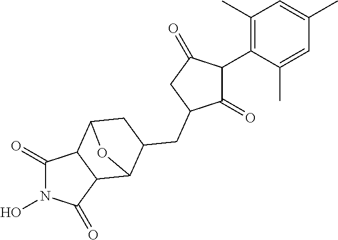 [SMILES: Cc1cc(C)c(C2C(=O)CC(CC3CC4OC3C3C(=O)N(O)C(=O)C43)C2=O)c(C)c1] | LC-MS (Method A) ES⁺: MH⁺ = 412 rt = 1.14 min |
| T130 | 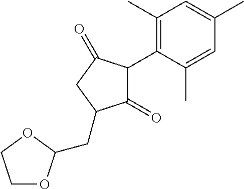 [SMILES: Cc1cc(C)c(C2C(=O)CC(CC3OCCO3)C2=O)c(C)c1] | LC-MS (Method A) ES⁺: MH⁺ = 303 rt = 1.26 min |
| T131 | 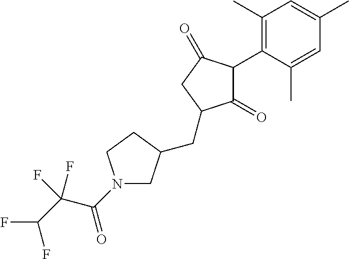 [SMILES: Cc1cc(C)c(C2C(=O)CC(CC3CCN(C(=O)C(F)(F)C(F)F)C3)C2=O)c(C)c1] | LC-MS (Method A) ES⁺: MH⁺ = 428 rt = 1.46 min |
| T132 | 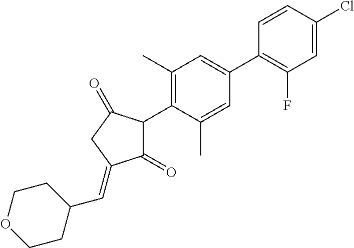 [SMILES: Cc1cc(-c2ccc(Cl)cc2F)cc(C)c1C1C(=O)C/C(=C\C2CCOCC2)C1=O] | LC-MS (Method C) ES⁻: M − H⁺ = 425, 427 rt = 4.50 mins |
| T133 | 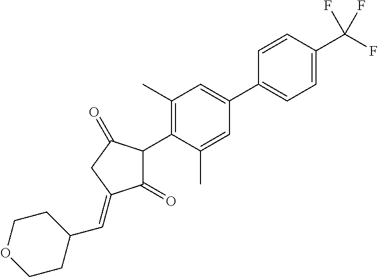 [SMILES: Cc1cc(-c2ccc(C(F)(F)F)cc2)cc(C)c1C1C(=O)C/C(=C\C2CCOCC2)C1=O] | LC-MS (Method C) ES⁻: M − H⁺ = 441 rt = 4.47 mins |
| T134 | 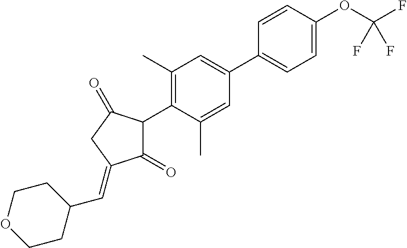 [SMILES: Cc1cc(-c2ccc(OC(F)(F)F)cc2)cc(C)c1C1C(=O)C/C(=C\C2CCOCC2)C1=O] | □□ ppm □ 1.64-1.62 (m, 4H), 2.5 (m, 1H), 3.16 (s, 2H), 3.52-3.46 (m, 2H), 4.02-3.98 (m, 2H), 6.03 (d, 1H), 6.26 (s, 1H), 7.29-7.25 (m, 4H), 7.56 (d, 2H). |
| T135 | 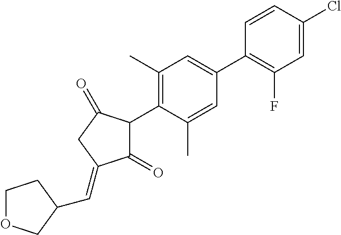 [SMILES: Cc1cc(-c2ccc(Cl)cc2F)cc(C)c1C1C(=O)C/C(=C\C2CCOC2)C1=O] | Melting point: 135-137° C. |
| T136 | 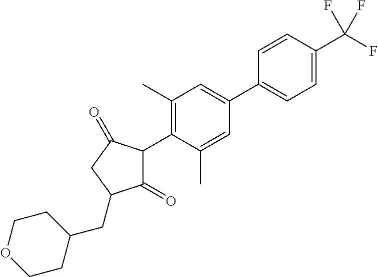 [SMILES: Cc1cc(-c2ccc(C(F)(F)F)cc2)cc(C)c1C1C(=O)CC(CC2CCOCC2)C1=O] | LC-MS (Method C) ES⁺: MH⁺ = 445 rt = 4.43 mins □ |
| T137 | 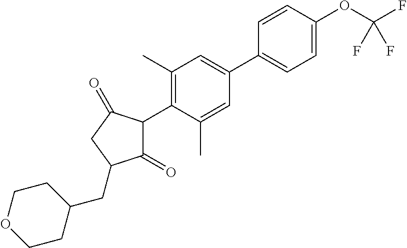 [SMILES: Cc1cc(-c2ccc(OC(F)(F)F)cc2)cc(C)c1C1C(=O)CC(CC2CCOCC2)C1=O] | □□□ CD₃OD □□ ppm □ 1.8-1.5 (m, 6H), 2.07 (s, 3H), 2.08 (s, 3H), 2.67-2.59 (m, 2H), 3.2 (m, 2H), 3.35 (m, 2H), 3.9 (m, 2H), 7.22 (s, 2H), 7.58 (d, 2H), 7.67 (d, 2H). |
| T138 | 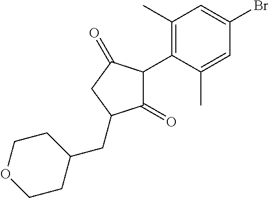 [SMILES: Cc1cc(Br)cc(C)c1C1C(=O)CC(CC2CCOCC2)C1=O] | □□ ppm □ 1.33 (m, 3H), 1.66 (m, 3H), 1.88 (m, 1H), 2.05 (s, 6H), 2.29 (m, 1H), 2.78 (m, 2H), 3.38 (m, 2H), 3.9 (m, 2H), 7.21 (s, 2H). |
| T139 | 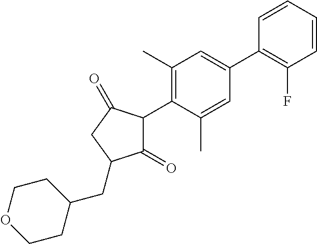 [SMILES: Cc1cc(-c2ccccc2F)cc(C)c1C1C(=O)CC(CC2CCOCC2)C1=O] | □□ ppm □ 1.4 (m, 4H), 1.7 (m, 2H), 1.9 (m, 1H), 2.18 (2 × s, 6H), 2.4 (br, 1H), 2.9 (br, 2H), 3.4 (m, 2H), 3.97 (m, 2H), 7.14 (m, 1H), 7.2 (m, 1H), 7.28 (s, 2H), 7.32 (d, 1H), 7.4 (m, 1H). |
| T140 | 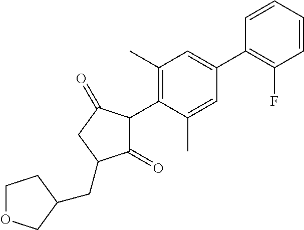 [SMILES: Cc1cc(-c2ccccc2F)cc(C)c1C1C(=O)CC(CC2CCOC2)C1=O] | LC-MS (Method C) ES⁺: MH⁺ = 380 rt = 3.98 min □ |
| T141 | 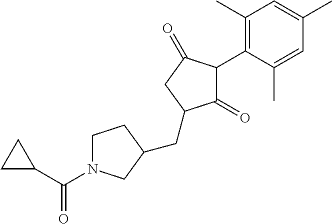 [SMILES: Cc1cc(C)c(C2C(=O)CC(CC3CCN(C(=O)C4CC4)C3)C2=O)c(C)c1] | LC-MS (Method A) ES⁺: MH⁺ = 368 rt = 1.29 min |
| T142 | 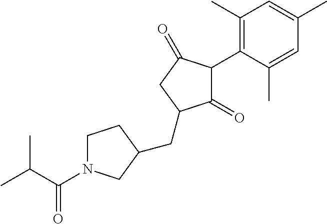 [SMILES: Cc1cc(C)c(C2C(=O)CC(CC3CCN(C(=O)C(C)C)C3)C2=O)c(C)c1] | LC-MS (Method A) ES⁺: MH⁺ = 370 rt = 1.39 min |
| T143 | 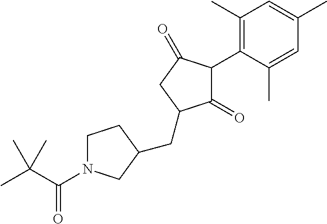 [SMILES: Cc1cc(C)c(C2C(=O)CC(CC3CCN(C(=O)C(C)(C)C)C3)C2=O)c(C)c1] | LC-MS (Method A) ES⁺: MH⁺ = 384 rt = 1.39 min |
| T144 | 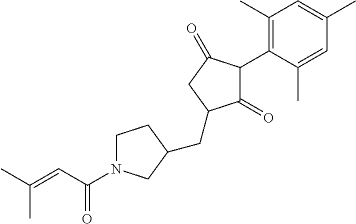 [SMILES: CC(C)=CC(=O)N1CCC(CC2CC(=O)C(c3c(C)cc(C)cc3C)C2=O)C1] | LC-MS (Method A) ES⁺: MH⁺ = 382 rt = 1.36 min |
| T145 | 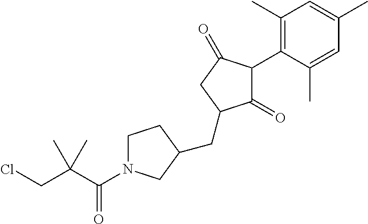 [SMILES: Cc1cc(C)c(C2C(=O)CC(CC3CCN(C(=O)C(C)(C)CCl)C3)C2=O)c(C)c1] | LC-MS (Method A) ES⁺: MH⁺ = 420, 418 rt = 1.41 min |
| T146 | 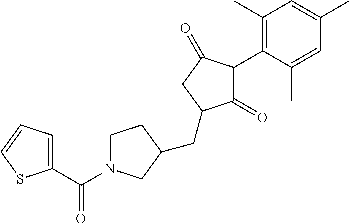 [SMILES: Cc1cc(C)c(C2C(=O)CC(CC3CCN(C(=O)c4cccs4)C3)C2=O)c(C)c1] | LC-MS (Method A) ES⁺: MH⁺ = 410 rt = 1.38 min |
| T147 | 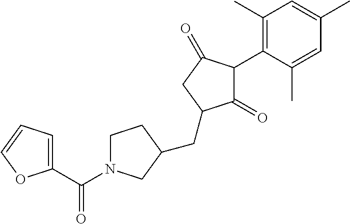 [SMILES: Cc1cc(C)c(C2C(=O)CC(CC3CCN(C(=O)c4ccco4)C3)C2=O)c(C)c1] | LC-MS (Method A) ES⁺: MH⁺ = 394 rt = 1.31 min |
| T148 | 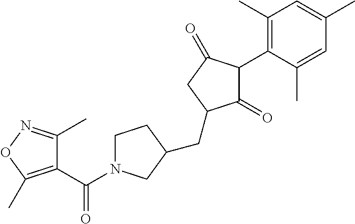 [SMILES: Cc1cc(C)c(C2C(=O)CC(CC3CCN(C(=O)c4c(C)noc4C)C3)C2=O)c(C)c1] | LC-MS (Method A) ES⁺: MH⁺ = 423 rt = 1.27 min |
| T149 | 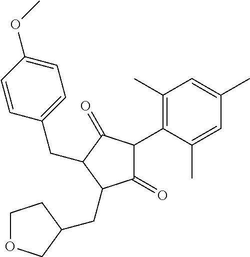 [SMILES: COc1ccc(CC2C(=O)C(c3c(C)cc(C)cc3C)C(=O)C2CC2CCOC2)cc1] | LC-MS (Method A) ES⁺: MH⁺ = 421 rt = 1.65 min |
| T150 | 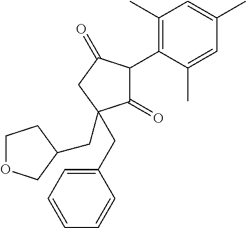 [SMILES: Cc1cc(C)c(C2C(=O)CC(Cc3ccccc3)(CC3CCOC3)C2=O)c(C)c1] | LC-MS (Method A) ES⁺: MH⁺ = 391 rt = 1.62 min |
| T151 | 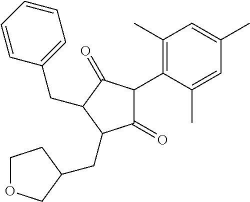 [SMILES: Cc1cc(C)c(C2C(=O)C(Cc3ccccc3)C(CC3CCOC3)C2=O)c(C)c1] | LC-MS (Method A) ES⁺: MH⁺ = 391 rt = 1.65 min |
| T152 | 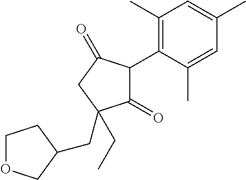 [SMILES: CCC1(CC2CCOC2)CC(=O)C(c2c(C)cc(C)cc2C)C1=O] | LC-MS (Method A) ES⁺: MH⁺ = 329 rt = 1.45 min |
| T153 | 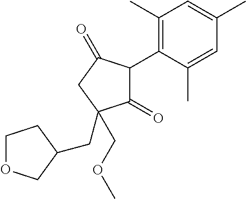 [SMILES: COCC1(CC2CCOC2)CC(=O)C(c2c(C)cc(C)cc2C)C1=O] | LC-MS (Method A) ES⁺: MH⁺ = 345 rt = 1.38 min |
| T154 | 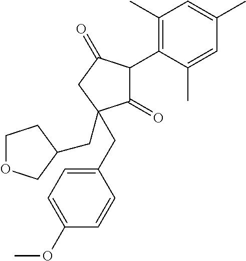 [SMILES: COc1ccc(CC2(CC3CCOC3)CC(=O)C(c3c(C)cc(C)cc3C)C2=O)cc1] | LC-MS (Method A) ES⁺: MH⁺ = 421 rt = 1.60 min |
| T155 | 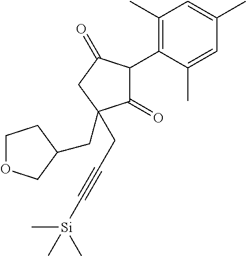 [SMILES: Cc1cc(C)c(C2C(=O)CC(CC#C[Si](C)(C)C)(CC3CCOC3)C2=O)c(C)c1] | LC-MS (Method A) ES⁺: MH⁺ = 411 rt = 1.81 min |
| T156 | 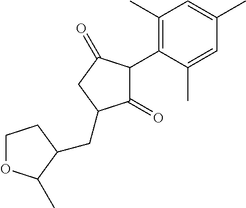 [SMILES: Cc1cc(C)c(C2C(=O)CC(CC3CCOC3C)C2=O)c(C)c1] | LC-MS (Method A) ES⁺: MH⁺ = 315 rt = 1.37 min |
| T157 | 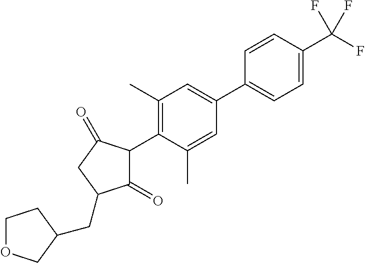 [SMILES: Cc1cc(-c2ccc(C(F)(F)F)cc2)cc(C)c1C1C(=O)CC(CC2CCOC2)C1=O] | □□ ppm 1.6 (m, 2H), 2.10 (m, 2H), 2.20 (s, 6H), 2.40 (m, 2H), 2.90 (br, 2H), 3.40 (m, 1H), 3.76 (m, 1H), 3.90 (m, 2H), 7.31 (s, 2H), 7.66 (m, 4H). |
| T158 | 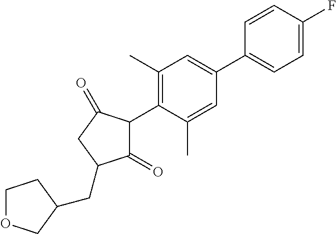 [SMILES: Cc1cc(-c2ccc(F)cc2)cc(C)c1C1C(=O)CC(CC2CCOC2)C1=O] | □□ ppm 1.60 (m, 2H), □ 2.05 (m, 2H), 2.10 (s, 6H), 2.40 (m, 2H), 2.90 (m, 2H), 3.40 (m, 1H), 3.78 (m, 1H), 3.9 (m, 2H), 7.10 (t, 2H), 7.25 (s, 2H), 7.5 (m, 2H). |
| T159 | 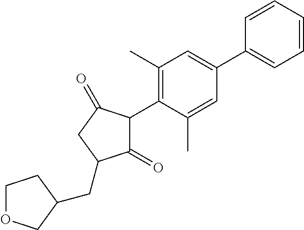 [SMILES: Cc1cc(-c2ccccc2)cc(C)c1C1C(=O)CC(CC2CCOC2)C1=O] | □□ ppm □ 1.4 (br, 2H), 1.9 (br, 2H), 2.09 (s, 3H), 2.1 (d, 3H), 2.22 (d, 2H), 2.6 (m, 2H), 3.2 (m, 1H), 3.62 (m, 1H), 3.76 (m, 2H), 7.25 (s, 2H), 7.32 (d, 1H), 7.39 (t, 2H), 7.52 (d, 2H). |
| T160 | 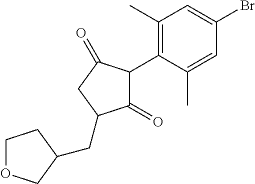 [SMILES: Cc1cc(Br)cc(C)c1C1C(=O)CC(CC2CCOC2)C1=O] | □□ ppm □ 1.33 (m, 3H), 1.4 (m, 1H), 1.55 (m, 1H), 1.95 (m, 1H), 2.01 (s, 3H), 2.03 (s, 3H), 2.10 (m, 1H), 2.29-2.17 (m, 2H), 2.75-2.57 (m, 2H), 3.3 (m, 1H), 3.69 (m, 1H), 3.82 (m, 2H), 7.17 (s, 2H), |
| T161 | 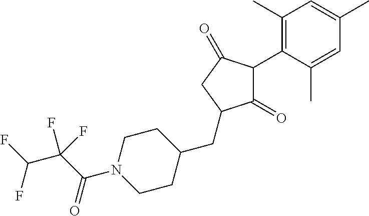 [SMILES: Cc1cc(C)c(C2C(=O)CC(CC3CCN(C(=O)C(F)(F)C(F)F)CC3)C2=O)c(C)c1] | LC-MS (Method A) ES⁺: MH⁺ = 442 rt = 1.66 min |
| T162 | 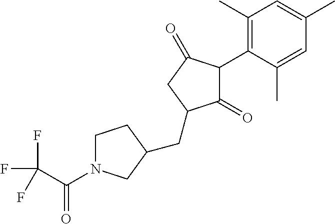 [SMILES: Cc1cc(C)c(C2C(=O)CC(CC3CCN(C(=O)C(F)(F)F)C3)C2=O)c(C)c1] | LC-MS (Method A) ES⁺: MH⁺ = 396 rt = 1.51 min |
| T163 | 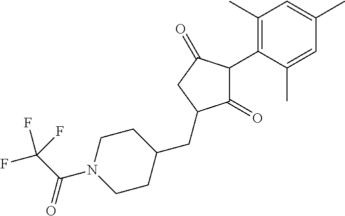 [SMILES: Cc1cc(C)c(C2C(=O)CC(CC3CCN(C(=O)C(F)(F)F)CC3)C2=O)c(C)c1] | LC-MS (Method A) ES⁺: MH⁺ = 410 rt = 1.59 min |
| T164 | 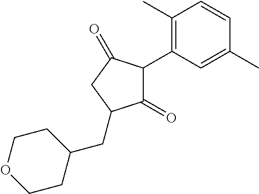 [SMILES: Cc1ccc(C)c(C2C(=O)CC(CC3CCOCC3)C2=O)c1] | LC-MS (Method A) ES⁺: MH⁺ = 301 rt = 1.31 min |
| T165 | 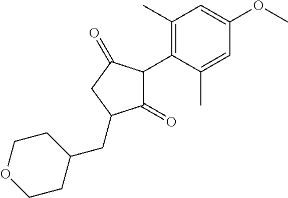 [SMILES: COc1cc(C)c(C2C(=O)CC(CC3CCOCC3)C2=O)c(C)c1] | LC-MS (Method A) ES⁺: MH⁺ = 331 rt = 1.27 min |
| T166 | 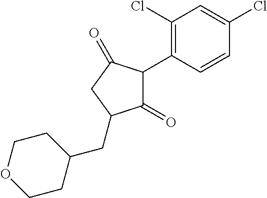 [SMILES: O=C1CC(CC2CCOCC2)C(=O)C1c1ccc(Cl)cc1Cl] | LC-MS (Method A) ES⁺: MH⁺ = 341, 343, 345 rt = 1.39 min |
| T167 | 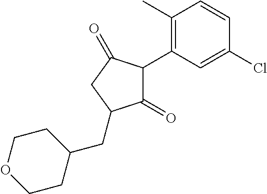 [SMILES: Cc1ccc(Cl)cc1C1C(=O)CC(CC2CCOCC2)C1=O] | LC-MS (Method A) ES⁺: MH⁺ = 321, 323 rt = 1.39 min |
| T168 | 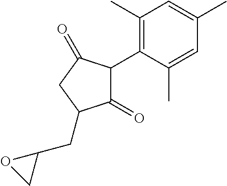 [SMILES: Cc1cc(C)c(C2C(=O)CC(CC3CO3)C2=O)c(C)c1] | LC-MS (Method A) ES⁺: MH⁺ = 273 rt = 1.26 min |
| T169 | 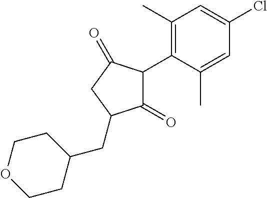 [SMILES: Cc1cc(Cl)cc(C)c1C1C(=O)CC(CC2CCOCC2)C1=O] | □□ ppm □ 1.33 (m, 3H), 1.65 (m, 3H), 1.9 (m, 1H), 2.08 (s, 3H), 2.09 (s, 3H), 2.35 (d, 1H), 2.85 (d, 2H), 3.39 (m, 2H), 3.97 (m, 2H), 7.09 (s, 2H). |
| T170 | 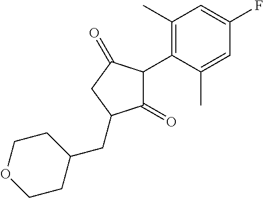 [SMILES: Cc1cc(F)cc(C)c1C1C(=O)CC(CC2CCOCC2)C1=O] | □□ ppm □ 1.42 (m, 3H), 1.70 (m, 3H), 1.89 (m, 1H), 2.08 (s, 3H), 2.10 (s, 3H), 2.34 (d, 1H), 2.85 (d, 2H), 3.39 (m, 2H), 3.97 (m, 2H), 6.8 (d, 2H). |
| T171 | 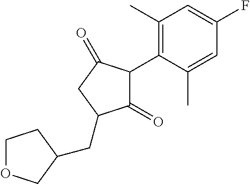 [SMILES: Cc1cc(F)cc(C)c1C1C(=O)CC(CC2CCOC2)C1=O] | LC-MS (Method C) ES⁺: MH⁺ = 305 rt = 3.37 min |
| T172 | 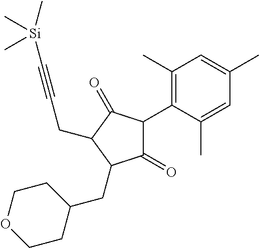 | LC-MS (Method A) ES⁺: MH⁺ = 425 rt = 1.83 min |
| T173 | 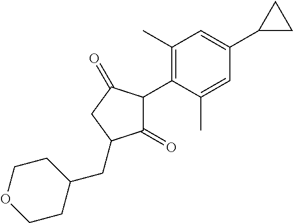 [SMILES: Cc1cc(C2CC2)cc(C)c1C1C(=O)CC(CC2CCOCC2)C1=O] | □□ ppm □ 0.65 (m, 2H), 0.9 (m, 2H), 1.41 (m, 3H), 1.68 (m, 3H), 1.80 (m, 1H), 1.90 (m, 1H), 2.07 (s, 6H), 2.35 (br, 1H), 2.8 (br, 2H), 3.38 (m, 2H), 3.96 (m, 2H), 6.78 (s, 2H) |
| T174 | 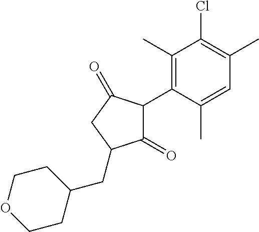 [SMILES: Cc1cc(C)c(C2C(=O)CC(CC3CCOCC3)C2=O)c(C)c1Cl] | LC-MS (Method A) ES⁺: MH⁺ = 349, 351 rt = 1.48 min |
| T175 | 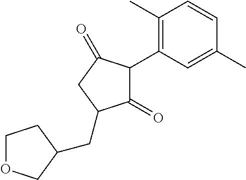 [SMILES: Cc1ccc(C)c(C2C(=O)CC(CC3CCOC3)C2=O)c1] | LC-MS (Method A) ES⁺: MH⁺ = 287 rt = 1.24 min |
| T176 | 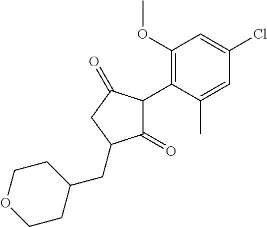 [SMILES: COc1cc(Cl)cc(C)c1C1C(=O)CC(CC2CCOCC2)C1=O] | LC-MS (Method A) ES⁺: MH⁺ = 351, 353 rt = 1.35 min |
| T177 | 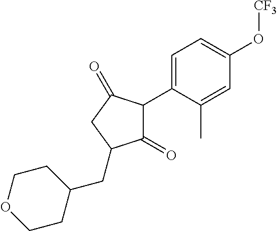 [SMILES: Cc1cc(OC(F)(F)F)ccc1C1C(=O)CC(CC2CCOCC2)C1=O] | LC-MS (Method A) ES⁺: MH⁺ = 371. rt = 1.51 min |
| T178 | 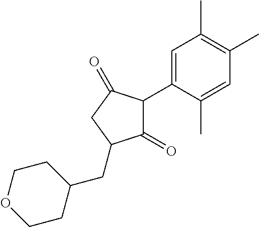 [SMILES: Cc1cc(C)c(C2C(=O)CC(CC3CCOCC3)C2=O)cc1C] | LC-MS (Method A) ES⁺: MH⁺ = 315. rt = 1.31 min |

TABLE P1

| Compound | | ¹H nmr (CDCl₃ unless stated), or other |
|---|---|---|
| Number | Structure | physical data |
| P1 | 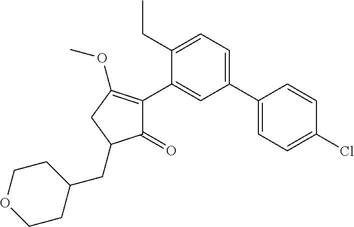 [SMILES: CCc1ccc(-c2ccc(Cl)cc2)cc1C1=C(OC)CC(CC2CCOCC2)C1=O] | □ ppm 1.21 (t, 3H), 1.32-1.47 (m, 3H), 1.61-1.70 (m, 2H), 1.72-1.82 (m, 1H), 1.91-2.02 (m, 1H), 2.42-2.58 (m, 3H), 2.65-2.77 (m, 1H), 3.00 (dd, 1H), 3.35-3.45 (m, 2H), 3.84 (s, 3H), 3.95- 4.04 (m, 2H), 7.24 (d, 1H), 7.38-7.41 (m, 2H), 7.42 (d, 1H), 7.45-7.49 (m, 2H). 7.55 (dd, 1H) |
| P2 | 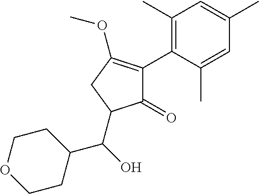 [SMILES: COC1=C(c2c(C)cc(C)cc2C)C(=O)C(C(O)C2CCOCC2)C1] | □ ppm 1.53 (d, 2H), 1.70-1.79 (m, 2H), 1.85 (td, 1 H), 2.09 (d, 6H), 2.26 (s, 3H), 2.50 (dd, 1H), 2.73-2.81 (m, 1H), 2.83-2.94 (m, 1H), 3.33-3.48 (m, 2H), 3.61 (d, 1H), 3.74 (s, 3H), 4.01- 4.10 (m, 2H), 4.58 (s, 1H), 6.87 (s, 2H) |
| P3 | 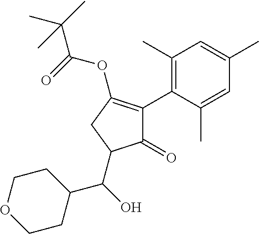 [SMILES: Cc1cc(C)c(C2=C(OC(=O)C(C)(C)C)CC(C(O)C3CCOCC3)C2=O)c(C)c1] | □ ppm 1.11 (s, 9H), 1.53 (d, 2H), 1.69-1.91 (m, 3H), 2.06 (d, 6H), 2.26 (s, 3H), 2.78-2.86 (m, 3H), 2.89 (ddd, 1H), 3.13 (dd, 1H), 3.33-3.47 (m, 2H), 3.69 (d, 1H), 4.05 (td, 2H), 4.13 (s, 3H), 6.85 (s, 2H) |
| P4 | 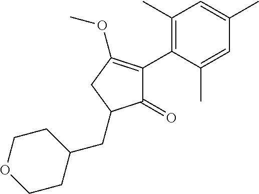 [SMILES: COC1=C(c2c(C)cc(C)cc2C)C(=O)C(CC2CCOCC2)C1] | □ ppm 1.28-1.46 (m, 3H), 1.61-1.70 (m, 2H), 1.70-1.79 (m, 1H), 1.94 (ddd, 1H), 2.08 (d, 6H), 2.26 (s, 3H), 2.44 (dd, 1H), 2.66-2.77 (m, 1H), 2.97 (dd, 1H), 3.35-3.48 (m, 2H), 3.73 (s, 3H), 3.95-4.05 (m, 2H), 6.87 (s, 2H) |
| P5 | 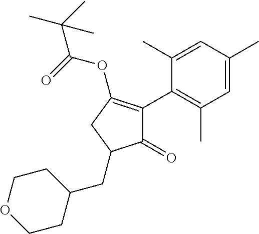 [SMILES: Cc1cc(C)c(C2=C(OC(=O)C(C)(C)C)CC(CC3CCOCC3)C2=O)c(C)c1] | □ ppm 1.09 (s, 9H), 1.28-1.46 (m, 3H), 1.59-1.69 (m, 2H), 1.70-1.77 (m, 1H), 1.94 (ddd, 1H), 2.05 (d, 6H), 2.25 (s, 3H), 2.70 (dd, 1H), 2.76-2.86 (m, 1H), 3.16 (dd, 1H), 3.33-3.45 (m, 2H), 3.91-4.07 (m, 2H), 6.84 (s, 2H) |
| P6 | 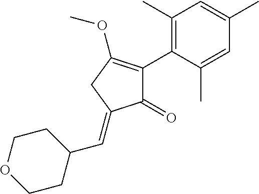 [SMILES: COC1=C(c2c(C)cc(C)cc2C)C(=O)/C(=C/C2CCOCC2)C1] | □ ppm 1.58-1.68 (m, 4H), 2.12 (s, 6H), 2.27 (s, 3H), 2.37-2.54 (m, 1 H), 3.09 (d, 2H), 3.49 (td, 2H), 3.55 (s, 3H), 3.96-4.07 (m, 2H), 5.92 (d, 1 H), 6.88 (s, 2H) |
| P7 | 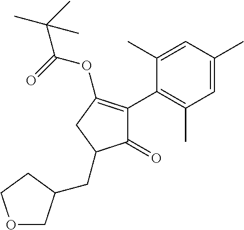 [SMILES: Cc1cc(C)c(C2=C(OC(=O)C(C)(C)C)CC(CC3CCOC3)C2=O)c(C)c1] | □ ppm 1.09 (s, 9H), 1.58-1.66 (m, 2H), 2.05 (d, 6H), 2.07-2.15 (m, 2H), 2.25 (s, 3H), 2.35-2.45 (m, 1 H), 2.64- 2.79 (m, 2H), 3.17 (dd, 1H), 3.40 (dt, 1H), 3.79 (ddd, 1H), 3.89 (ddd, 1H), 3.97 (t, 1H), 6.84 (s, 2H) |
| P8 | 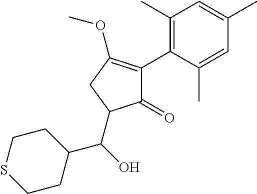 [SMILES: COC1=C(c2c(C)cc(C)cc2C)C(=O)C(C(O)C2CCSCC2)C1] | □ ppm 1.44-1.52 (m, 1H), 1.79 (ddd, 1H), 1.88-1.95 (m, 1H), 1.96-2.03 (m, 2H), 2.08 (d, 6H), 2.26 (s, 3H), 2.44 (dd, 1 H), 2.66-2.74 (m, 4H), 2.75-2.81 (m, 1H), 2.82-2.92 (m, 1H), 3.59 (dd, 1H), 3.74 (s, 3H), 4.75 (s, 1H), 6.87 (s, 2H) |
| P9 | 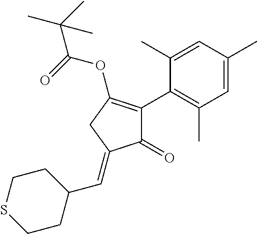 [SMILES: Cc1cc(C)c(C2=C(OC(=O)C(C)(C)C)C/C(=C\C3CCSCC3)C2=O)c(C)c1] | □ ppm 1.11 (s, 9H), 1.70 (ddd, 2H), 1.97-2.06 (m, 2H), 2.07 (s, 6H), 2.26 (s, 3H), 2.28-2.40 (m, 1H), 2.62-2.80 (m, 4H), 3.59 (d, 2H), 6.57 (d, 1H), 6.85 (s, 2H) |
| P10 | 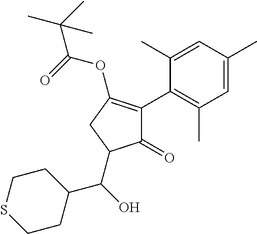 [SMILES: Cc1cc(C)c(C2=C(OC(=O)C(C)(C)C)CC(C(O)C3CCSCC3)C2=O)c(C)c1] | □ ppm 1.11 (s, 9H), 1.48-1.53 (m, 1H), 1.74-1.84 (m, 1H), 1.88-1.95 (m, 1H), 1.96-2.02 (m, 2H), 2.05 (d, 6H), 2.26 (s, 3H), 2.63-2.73 (m, 4H), 2.74- 2.82 (m, 1H), 2.90 (ddd, 1H), 3.10 (dd, 1H), 3.67 (dd, 1H), 4.32 (s, 1H), 6.85 (s, 2H) |
| P11 | 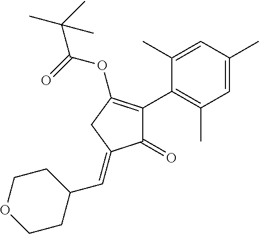 [SMILES: Cc1cc(C)c(C2=C(OC(=O)C(C)(C)C)C/C(=C\C3CCOCC3)C2=O)c(C)c1] | LC-MS (Method A) ES⁺: MH⁺ = 397 rt = 1.95 min |
| P12 | 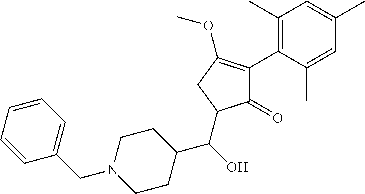 | LC-MS (Method A) ES⁺ : MH⁺ = 315 rt = 1.37 min |
| P13 | 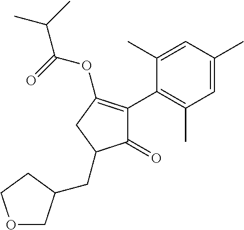 [SMILES: Cc1cc(C)c(C2=C(OC(=O)C(C)C)CC(CC3CCOC3)C2=O)c(C)c1] | LC-MS (Method A) ES⁺: MH⁺ = 371 rt = 1.83 min |
| P14 | 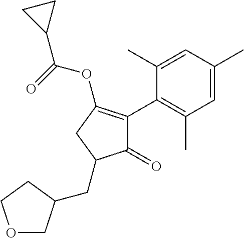 [SMILES: Cc1cc(C)c(C2=C(OC(=O)C3CC3)CC(CC3CCOC3)C2=O)c(C)c1] | LC-MS (Method A) ES⁺: MH⁺ = 369 rt =1.73 min |
| P15 | 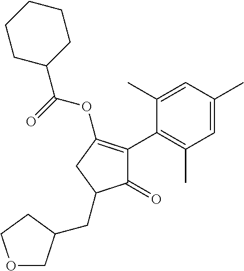 [SMILES: Cc1cc(C)c(C2=C(OC(=O)C3CCCCC3)CC(CC3CCOC3)C2=O)c(C)c1] | LC-MS (Method A) ES⁺: MH⁺ = 411 rt = 1.95 min |
| P16 | 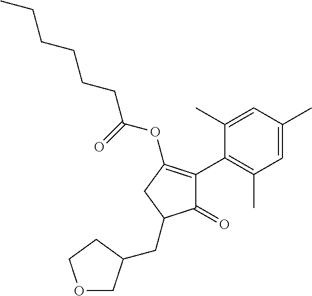 [SMILES: CCCCCCC(=O)OC1=C(c2c(C)cc(C)cc2C)C(=O)C(CC2CCOC2)C1] | LC-MS (Method A) ES⁺: MH⁺ = 413 rt = 2.00 min |
| P17 | 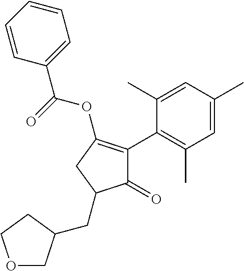 [SMILES: Cc1cc(C)c(C2=C(OC(=O)c3ccccc3)CC(CC3CCOC3)C2=O)c(C)c1] | LC-MS (Method A) ES⁺: MH⁺ = 405 rt = 1.81 min |
| P18 | 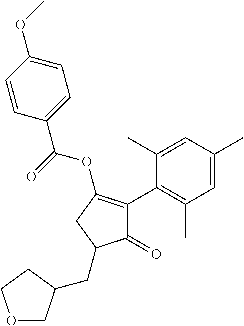 [SMILES: COc1ccc(C(=O)OC2=C(c3c(C)cc(C)cc3C)C(=O)C(CC3CCOC3)C2)cc1] | LC-MS (Method A) ES⁺: MH⁺ = 435 rt = 1.81 min |
| P19 | 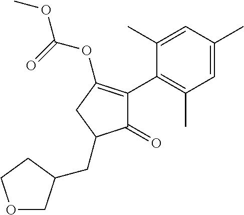 [SMILES: COC(=O)OC1=C(c2c(C)cc(C)cc2C)C(=O)C(CC2CCOC2)C1] | LC-MS (Method A) ES⁺: MH⁺ = 359 rt = 1.56 min |
| P20 | 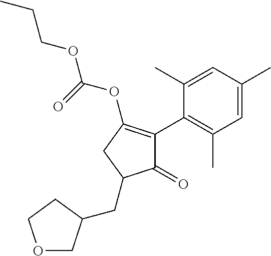 [SMILES: CCCOC(=O)OC1=C(c2c(C)cc(C)cc2C)C(=O)C(CC2CCOC2)C1] | LC-MS (Method A) ES⁺: MH⁺ = 387 |
| P21 | 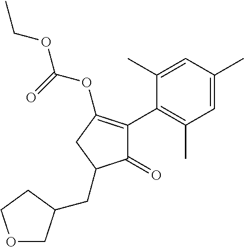 [SMILES: CCOC(=O)OC1=C(c2c(C)cc(C)cc2C)C(=O)C(CC2CCOC2)C1] | LC-MS (Method A) ES⁺: MH⁺ = 373 |
| P22 | 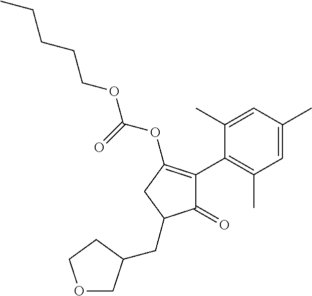 [SMILES: CCCCCOC(=O)OC1=C(c2c(C)cc(C)cc2C)C(=O)C(CC2CCOC2)C1] | LC-MS (Method A) ES⁺: MH⁺ = 415 |
| P23 | 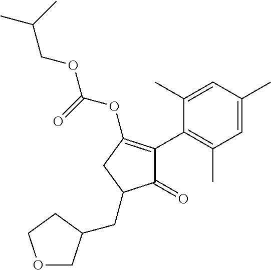 [SMILES: Cc1cc(C)c(C2=C(OC(=O)OCC(C)C)CC(CC3CCOC3)C2=O)c(C)c1] | LC-MS (Method A) ES⁺: MH⁺ = 401 |
| P24 | 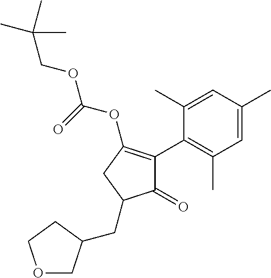 [SMILES: Cc1cc(C)c(C2=C(OC(=O)OCC(C)(C)C)CC(CC3CCOC3)C2=O)c(C)c1] | LC-MS (Method A) ES⁺: MH⁺ = 415 |
| P25 | 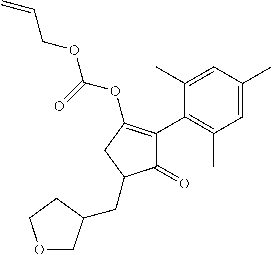 [SMILES: C=CCOC(=O)OC1=C(c2c(C)cc(C)cc2C)C(=O)C(CC2CCOC2)C1] | LC-MS (Method A) ES⁺: MH⁺ = 385 |
| P26 | 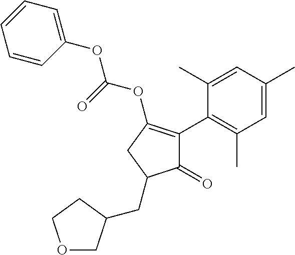 [SMILES: Cc1cc(C)c(C2=C(OC(=O)Oc3ccccc3)CC(CC3CCOC3)C2=O)c(C)c1] | LC-MS (Method A) ES⁺: MH⁺ = 421 rt = 1.83 min |
| P27 | 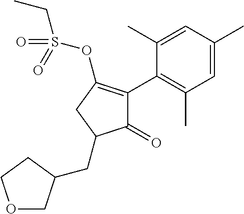 [SMILES: CCS(=O)(=O)OC1=C(c2c(C)cc(C)cc2C)C(=O)C(CC2CCOC2)C1] | LC-MS (Method A) ES⁺: MH⁺ = 393 rt = 1.64 min |
| P28 | 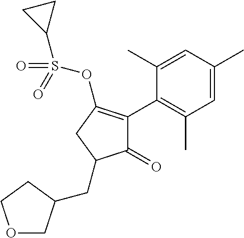 [SMILES: Cc1cc(C)c(C2=C(OS(=O)(=O)C3CC3)CC(CC3CCOC3)C2=O)c(C)c1] | LC-MS (Method A) ES⁺: MH⁺ = 405 rt = 1.66 min |
| P29 | 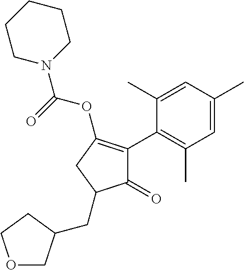 [SMILES: Cc1cc(C)c(C2=C(OC(=O)N3CCCCC3)CC(CC3CCOC3)C2=O)c(C)c1] | LC-MS (Method A) ES⁺: MH⁺ = 412 rt = 1.73 min |
| P30 | 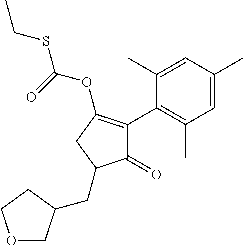 [SMILES: CCSC(=O)OC1=C(c2c(C)cc(C)cc2C)C(=O)C(CC2CCOC2)C1] | LC-MS (Method A) ES⁺: MH⁺ = 389 rt = 1.83 min |
| P31 | 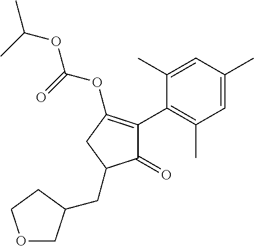 [SMILES: Cc1cc(C)c(C2=C(OC(=O)OC(C)C)CC(CC3CCOC3)C2=O)c(C)c1] | LC-MS (Method A) ES⁺: MH⁺ = 387 rt = 1.78 min |
| P32 | 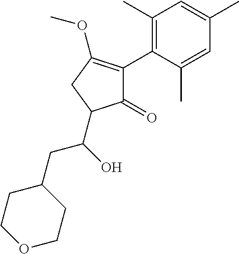 [SMILES: COC1=C(c2c(C)cc(C)cc2C)C(=O)C(C(O)CC2CCOCC2)C1] | LC-MS (Method B) ES⁺: MH⁺ = 359 rt = 1.26 min |
| P33 | 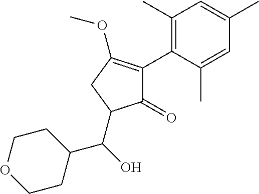 [SMILES: COC1=C(c2c(C)cc(C)cc2C)C(=O)C(C(O)C2CCOCC2)C1] | LC-MS (Method A) ES⁺: MH⁺ = 345 |
| P34 | 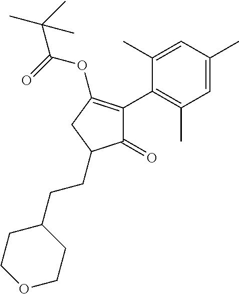 [SMILES: Cc1cc(C)c(C2=C(OC(=O)C(C)(C)C)CC(CCC3CCOCC3)C2=O)c(C)c1] | LC-MS (Method A) ES⁺: MH⁺ = 413 rt = 2.03 min |
| P35 | 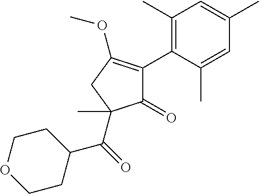 [SMILES: COC1=C(c2c(C)cc(C)cc2C)C(=O)C(C)(C(=O)C2CCOCC2)C1] | LC-MS (Method B) ES⁺: MH⁺ = 357 rt = 1.44 min |
| P36 | 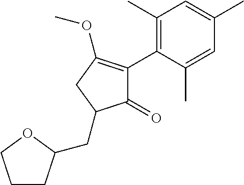 [SMILES: COC1=C(c2c(C)cc(C)cc2C)C(=O)C(CC2CCCO2)C1] | LC-MS (Method A) ES⁺: MH⁺ = 315 |
| P37 | 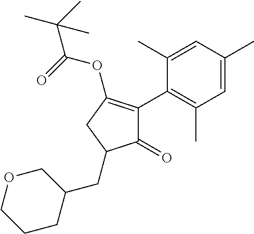 [SMILES: Cc1cc(C)c(C2=C(OC(=O)C(C)(C)C)CC(CC3CCCOC3)C2=O)c(C)c1] | LC-MS (Method A) ES⁺: M + H⁺ = 399 rt = 1.98 min |
| P38 | 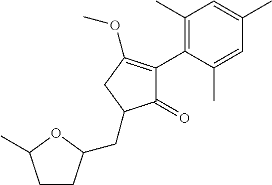 | LC-MS (Method A) ES⁺: MH⁺ = 329 |
| P39 | 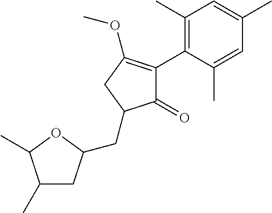 | LC-MS (Method A) ES⁺: MH⁺ = 342 |
| P40 | 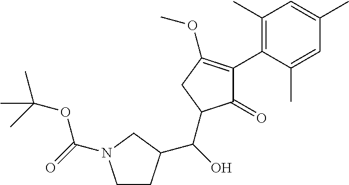 [SMILES: COC1=C(c2c(C)cc(C)cc2C)C(=O)C(C(O)C2CCN(C(=O)OC(C)(C)C)C2)C1] | LC-MS (Method A) ES⁺: MH⁺ = 430 |
| P41 | 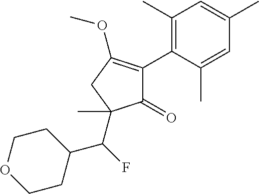 [SMILES: COC1=C(c2c(C)cc(C)cc2C)C(=O)C(C)(C(F)C2CCOCC2)C1] | LC-MS (Method A) ES⁺: MH⁺ = 361 rt = 1.54 min |
| P42 | 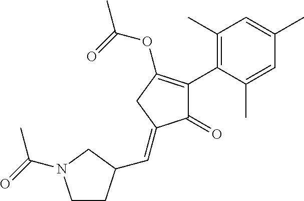 [SMILES: CC(=O)OC1=C(c2c(C)cc(C)cc2C)C(=O)/C(=C/C2CCN(C(C)=O)C2)C1] | LC-MS (Method A) ES⁺: MH⁺ = 382 |
| P43 | 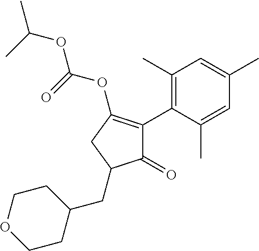 [SMILES: Cc1cc(C)c(C2=C(OC(=O)OC(C)C)CC(CC3CCOCC3)C2=O)c(C)c1] | LC-MS (Method A) ES⁺: MH⁺ = 401 rt = 1.77 min |
| P44 | 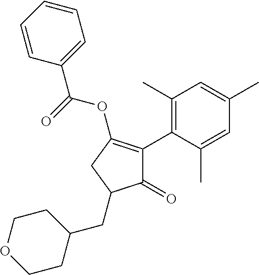 [SMILES: Cc1cc(C)c(C2=C(OC(=O)c3ccccc3)CC(CC3CCOCC3)C2=O)c(C)c1] | LC-MS (Method B) ES⁺: MH⁺ = 419 rt = 1.84 min |
| P45 | 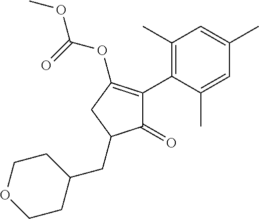 [SMILES: COC(=O)OC1=C(c2c(C)cc(C)cc2C)C(=O)C(CC2CCOCC2)C1] | LC-MS (Method A) ES⁺: MH⁺ = 373 rt = 1.64 min |
| P46 | 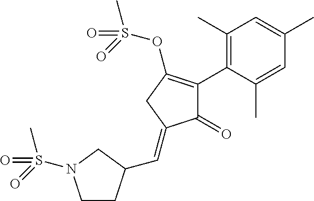 [SMILES: Cc1cc(C)c(C2=C(OS(C)(=O)=O)C/C(=C\C3CCN(S(C)(=O)=O)C3)C2=O)c(C)c1] | LC-MS (Method A) ES⁺: MH⁺ = 454 rt = 1.51 min |
| P47 | 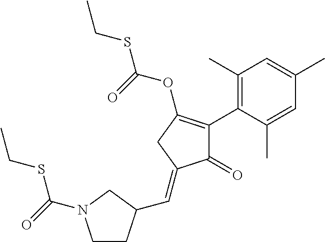 [SMILES: CCSC(=O)OC1=C(c2c(C)cc(C)cc2C)C(=O)/C(=C/C2CCN(C(=O)SCC)C2)C1] | LC-MS (Method A) ES⁺: MH⁺ = 474 rt = 1.91 min |
| P48 | 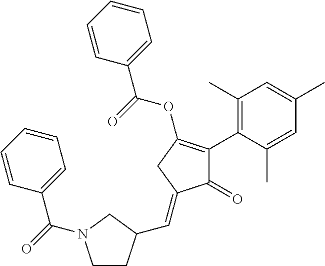 [SMILES: Cc1cc(C)c(C2=C(OC(=O)c3ccccc3)C/C(=C\C3CCN(C(=O)c4ccccc4)C3)C2=O)c(C)c1] | LC-MS (Method A) ES⁺: MH⁺ = 506 rt = 1.85 min |
| P49 | 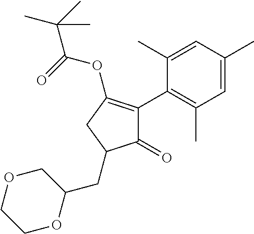 [SMILES: Cc1cc(C)c(C2=C(OC(=O)C(C)(C)C)CC(CC3COCCO3)C2=O)c(C)c1] | LC-MS (Method A) ES⁺: MH⁺ = 401 rt = 1.81 min |
| P50 | 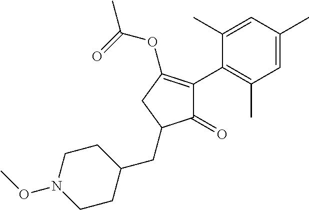 | LC-MS (Method A) ES⁺: MH⁺ = 386 rt = 1.53 min |
| P51 | 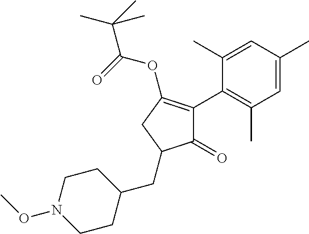 | LC-MS (Method A) ES⁺: MH⁺ = 428 rt = 1.88 min |
| P52 | 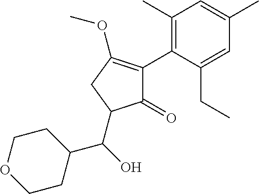 [SMILES: CCc1cc(C)cc(C)c1C1=C(OC)CC(C(O)C2CCOCC2)C1=O] | LC-MS (Method A) ES⁺: MH⁺ = 359 rt = 1.46 min |
| P53 | 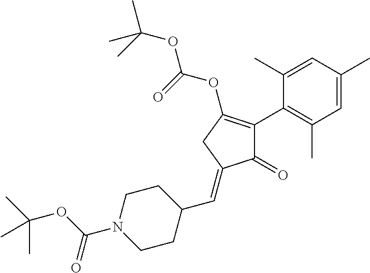 [SMILES: Cc1cc(C)c(C2=C(OC(=O)OC(C)(C)C)C/C(=C\C3CCN(C(=O)OC(C)(C)C)CC3)C2=O)c(C)c1] | LC-MS (Method A) ES⁺: MH⁺ = 512 rt = 2.03 min |
| P54 | 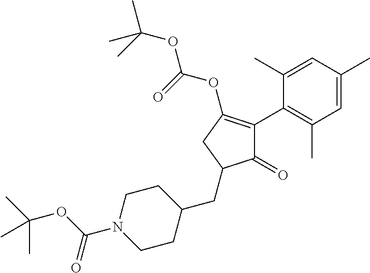 [SMILES: Cc1cc(C)c(C2=C(OC(=O)OC(C)(C)C)CC(CC3CCN(C(=O)OC(C)(C)C)CC3)C2=O)c(C)c1] | LC-MS (Method A) ES⁺: MH⁺ = 514 rt = 2.03 min |
| P55 | 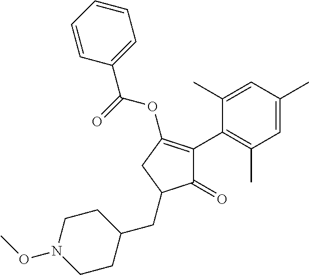 | LC-MS (Method A) ES⁺: MH⁺ = 448 rt = 1.78 min |
| P56 | 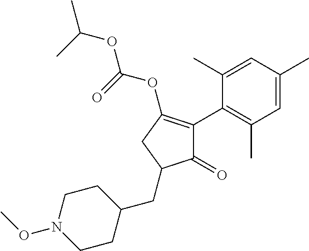 | LC-MS (Method A) ES⁺: MH⁺ = 430 rt = 1.64 min |
| P57 | 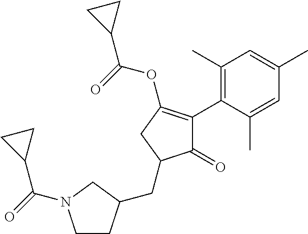 [SMILES: Cc1cc(C)c(C2=C(OC(=O)C3CC3)CC(CC3CCN(C(=O)C4CC4)C3)C2=O)c(C)c1] | LC-MS (Method A) ES⁺: MH⁺ = 436 rt = 1.56 mins |
| P58 | 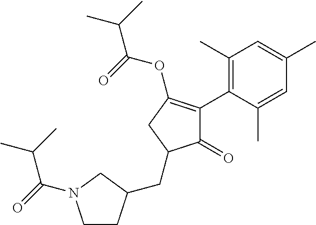 [SMILES: Cc1cc(C)c(C2=C(OC(=O)C(C)C)CC(CC3CCN(C(=O)C(C)C)C3)C2=O)c(C)c1] | LC-MS (Method A) ES⁺: MH⁺ = 440 rt = 1.71 mins |
| P59 | 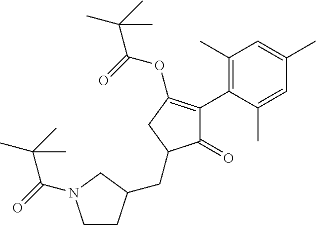 [SMILES: Cc1cc(C)c(C2=C(OC(=O)C(C)(C)C)CC(CC3CCN(C(=O)C(C)(C)C)C3)C2=O)c(C)c1] | LC-MS (Method A) ES⁺: MH⁺ = 468 rt = 1.85 mins |
| P60 | 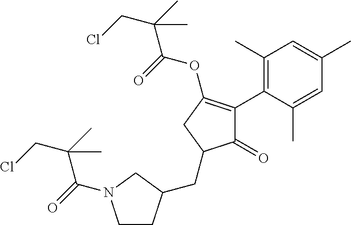 [SMILES: Cc1cc(C)c(C2=C(OC(=O)C(C)(C)CCl)CC(CC3CCN(C(=O)C(C)(C)CCl)C3)C2=O)c(C)c1] | LC-MS (Method A) ES⁺: MH⁺ = 540, 538, 536 rt = 1.85 mins |
| P61 | 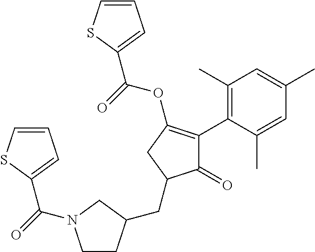 [SMILES: Cc1cc(C)c(C2=C(OC(=O)c3cccs3)CC(CC3CCN(C(=O)c4cccs4)C3)C2=O)c(C)c1] | LC-MS (Method A) ES⁺: MH⁺ = 520 rt = 1.76 mins |
| P62 | 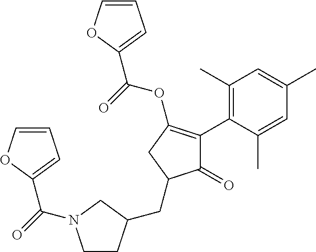 [SMILES: Cc1cc(C)c(C2=C(OC(=O)c3ccco3)CC(CC3CCN(C(=O)c4ccco4)C3)C2=O)c(C)c1] | LC-MS (Method A) ES⁺: MH⁺ = 488 rt = 1.63 mins |
| P63 | 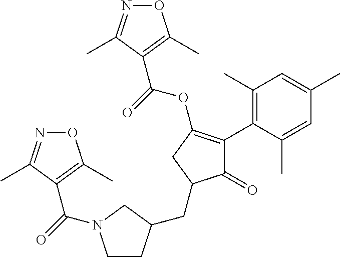 | LC-MS (Method A) ES⁺: MH⁺ = 546 rt = 1.66 mins |
| P64 | 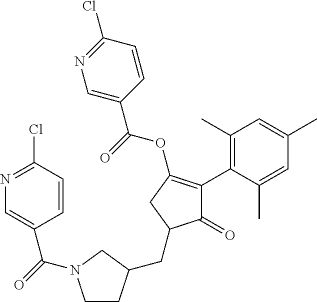 [SMILES: Cc1cc(C)c(C2=C(OC(=O)c3ccc(Cl)nc3)CC(CC3CCN(C(=O)c4ccc(Cl)nc4)C3)C2=O)c(C)c1] | LC-MS (Method A) ES⁺: MH⁺ = 582, 580, 578 rt = 1.86 mins |
| P65 | 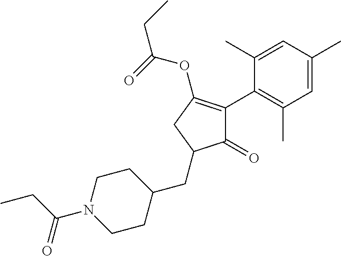 [SMILES: CCC(=O)OC1=C(c2c(C)cc(C)cc2C)C(=O)C(CC2CCN(C(=O)CC)CC2)C1] | LC-MS (Method A) ES⁺: MH⁺ = 426 rt = 1.78 mins |
| P66 | 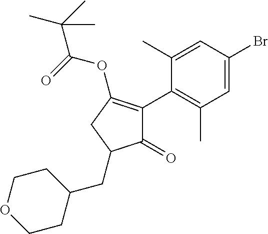 [SMILES: Cc1cc(Br)cc(C)c1C1=C(OC(=O)C(C)(C)C)CC(CC2CCOCC2)C1=O] | Melting point: 82-84° C. |
| P67 | 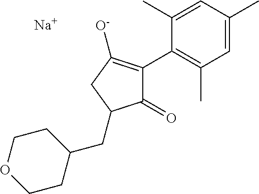 [SMILES: Cc1cc(C)c(C2=C([O-])CC(CC3CCOCC3)C2=O)c(C)c1 ; [Na+]] | □ ppm (D₂O) 1.20-1.40 (m, 3H), 1.60- 1.85 (m, 4H), 2.01 (s, 6H), 2.15-2.25 (m, 4H), 2.60-2.70 (m, 2H), 3.40-3.55 (m, 2H), 3.90-4.05 (m, 2H), 6.91 (s, 2H) |
| P68 | 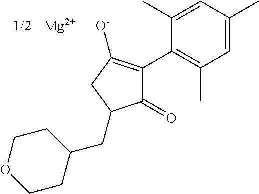 | □ ppm (D₂O) 1.20-1.40 (m, 3H), 1.60- 1.85 (m, 4H), 2.01 (s, 6H), 2.15-2.25 (m, 4H), 2.60-2.70 (m, 2H), 3.40-3.55 (m, 2H), 3.90-4.05 (m, 2H), 6.91 (s, 2H) |
| P69 | 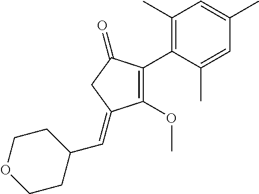 [SMILES: COC1=C(c2c(C)cc(C)cc2C)C(=O)C/C1=C\C1CCOCC1] | LC-MS (Method A) ES⁺: MH⁺ = 327 rt = 1.54 mins |
| P70 | 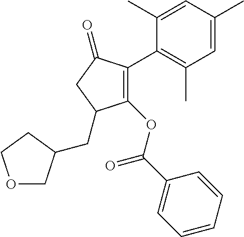 [SMILES: Cc1cc(C)c(C2=C(OC(=O)c3ccccc3)C(CC3CCOC3)CC2=O)c(C)c1] | LC-MS (Method A) ES⁺: MH⁺ = 405 rt = 1.75 mins |
| P71 | 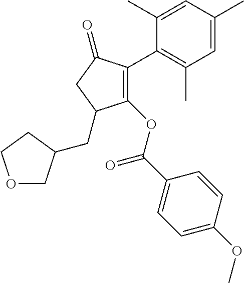 [SMILES: COc1ccc(C(=O)OC2=C(c3c(C)cc(C)cc3C)C(=O)CC2CC2CCOC2)cc1] | LC-MS (Method A) ES⁺: MH⁺ = 435 rt = 1.81 mins |

The compounds of the following Tables 1 to 102 can be obtained in an analogous manner.

Table 1 covers 262 compounds of the following type:

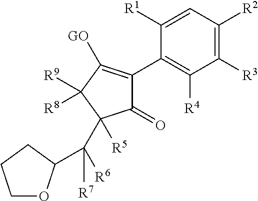

where G, R⁵, R⁶, R⁷, R⁸ and R⁹ are all hydrogen, and R¹, R², R³ and R⁴ are as described in Table 1 below:

| Compound | | | | |
|---|---|---|---|---|
| Number | R¹ | R² | R³ | R⁴ |
| 1.001 | CH₃ | H | H | H |
| 1.002 | CH₃ | CH₃ | H | H |
| 1.003 | CH₃ | H | CH₃ | H |
| 1.004 | CH₃ | H | H | CH₃ |
| 1.005 | CH₃ | CH₃ | CH₃ | H |
| 1.006 | CH₃ | CH₃ | H | CH₃ |
| 1.007 | CH₃ | CH₃ | CH₃ | CH₃ |
| 1.008 | CH₃ | Cl | H | H |
| 1.009 | CH₃ | Cl | H | CH₃ |
| 1.010 | CH₃ | Cl | H | OCH₃ |
| 1.011 | CH₃ | H | Cl | H |
| 1.012 | CH₃ | H | H | Cl |
| 1.013 | CH₃ | CH₃ | Cl | H |
| 1.014 | CH₃ | CH₃ | H | Cl |
| 1.015 | CH₃ | H | Cl | CH₃ |
| 1.016 | CH₃ | CH₃ | Cl | CH₃ |
| 1.017 | CH₃ | Br | H | H |
| 1.018 | CH₃ | Br | H | CH₃ |
| 1.019 | CH₃ | Br | H | OCH₃ |
| 1.020 | CH₃ | H | Br | H |
| 1.021 | CH₃ | H | H | Br |
| 1.022 | CH₃ | CH₃ | Br | H |
| 1.023 | CH₃ | CH₃ | H | Br |
| 1.024 | CH₃ | H | Br | CH₃ |
| 1.025 | CH₃ | CH₃ | Br | CH₃ |
| 1.026 | CH₃ | CH₃O | H | H |
| 1.027 | CH₃ | CH₃O | H | CH₃ |
| 1.028 | CH₃ | CH₃O | H | Cl |
| 1.029 | CH₃ | CH₃O | H | Br |
| 1.030 | CH₃ | CH₃CH₂O | H | H |
| 1.031 | CH₃ | CH₃CH₂O | H | CH₃ |
| 1.032 | CH₃ | CH₃CH₂O | H | Cl |
| 1.033 | CH₃ | CH₃CH₂O | H | Br |
| 1.034 | CH₃ | H | CH₃O | H |
| 1.035 | CH₃ | H | H | CH₃O |
| 1.036 | CH₃ | CH₃ | CH₃O | H |
| 1.037 | CH₃ | CH₃ | H | CH₃O |
| 1.038 | CH₃ | H | CH₃O | CH₃ |
| 1.039 | CH₃ | CH₃ | CH₃O | CH₃ |
| 1.040 | CH₃ | —CH═CH₂ | H | CH₃ |
| 1.041 | CH₃ | CH₃ | H | —CH═CH₂ |
| 1.042 | CH₃ | —C•CH | H | CH₃ |
| 1.043 | CH₃ | CH₃ | H | —C•CH |
| 1.044 | CH₃ | —CH═CH₂ | H | —CH═CH₂ |
| 1.045 | CH₃ | CH₂CH₃ | H | CH₃ |
| 1.046 | CH₃ | phenyl | H | CH₃ |
| 1.047 | CH₃ | 2-fluorophenyl | H | CH₃ |
| 1.048 | CH₃ | 2-chlorophenyl | H | CH₃ |
| 1.049 | CH₃ | 2-trifluoromethylphenyl | H | CH₃ |
| 1.050 | CH₃ | 2-nitrophenyl | H | CH₃ |
| 1.051 | CH₃ | 2-methylphenyl | H | CH₃ |
| 1.052 | CH₃ | 2- | H | CH₃ |
| | | methanesulfonylphenyl | | |
| 1.053 | CH₃ | 2-cyanophenyl | H | CH₃ |
| 1.054 | CH₃ | 3-fluorophenyl | H | CH₃ |
| 1.055 | CH₃ | 3-chlorophenyl | H | CH₃ |
| 1.056 | CH₃ | 3-trifluoromethylphenyl | H | CH₃ |
| 1.057 | CH₃ | 3-nitrophenyl | H | CH₃ |
| 1.058 | CH₃ | 3-methylphenyl | H | CH₃ |
| 1.059 | CH₃ | 3- | H | CH₃ |
| | | methanesulfonylphenyl | | |
| 1.060 | CH₃ | 3-cyanophenyl | H | CH₃ |
| 1.061 | CH₃ | 4-fluorophenyl | H | CH₃ |
| 1.062 | CH₃ | 4-chlorophenyl | H | CH₃ |
| 1.063 | CH₃ | 4-bromophenyl | H | CH₃ |
| 1.064 | CH₃ | 4- | H | CH₃ |
| | | difluoromethoxyphenyl | | |
| 1.065 | CH₃ | 2-fluoro-4-chlorophenyl | H | CH₃ |
| 1.066 | CH₃ | 2-chloro-4-chlorophenyl | H | CH₃ |
| 1.067 | CH₃ | 2-methyl-4- | H | CH₃ |
| | | chlorophenyl | | |
| 1.068 | CH₃ | 4-trifluoromethylphenyl | H | CH₃ |
| 1.069 | CH₃ | 4-nitrophenyl | H | CH₃ |
| 1.070 | CH₃ | 4-methylphenyl | H | CH₃ |
| 1.071 | CH₃ | 4- | H | CH₃ |
| | | methanesulfonylphenyl | | |
| 1.072 | CH₃ | 4-cyanophenyl | H | CH₃ |
| 1.073 | CH₃ | H | phenyl | H |
| 1.074 | CH₃ | H | 2-fluorophenyl | H |
| 1.075 | CH₃ | H | 2-chlorophenyl | H |
| 1.076 | CH₃ | H | 2-trifluoromethylphenyl | H |
| 1.077 | CH₃ | H | 2-nitrophenyl | H |
| 1.078 | CH₃ | H | 2-methylphenyl | H |
| 1.079 | CH₃ | H | 2-methylsulfonylphenyl | H |
| 1.080 | CH₃ | H | 2-cyanophenyl | H |
| 1.081 | CH₃ | H | 3-fluorophenyl | H |
| 1.082 | CH₃ | H | 3-chlorophenyl | H |
| 1.083 | CH₃ | H | 3-trifluoromethylphenyl | H |
| 1.084 | CH₃ | H | 3-nitrophenyl | H |
| 1.085 | CH₃ | H | 3-methylphenyl | H |
| 1.086 | CH₃ | H | 3-methylsulfonylphenyl | H |
| 1.087 | CH₃ | H | 3-cyanophenyl | H |
| 1.088 | CH₃ | H | 4-fluorophenyl | H |
| 1.089 | CH₃ | H | 4-chlorophenyl | H |
| 1.090 | CH₃ | H | 4-bromophenyl | H |
| 1.091 | CH₃ | H | 4- | H |
| | | | difluoromethoxyphenyl | |
| 1.092 | CH₃ | H | 2-fluoro-4-chlorophenyl | H |
| 1.093 | CH₃ | H | 2-chloro-4-chlorophenyl | H |
| 1.094 | CH₃ | H | 2-methyl-4- | H |
| | | | chlorophenyl | |
| 1.095 | CH₃ | H | 4-trifluoromethylphenyl | H |
| 1.096 | CH₃ | H | 4-nitrophenyl | H |
| 1.097 | CH₃ | H | 4-methylphenyl | H |
| 1.098 | CH₃ | H | 4-methylsulfonylphenyl | H |
| 1.099 | CH₃ | H | 4-cyanophenyl | H |
| 1.100 | CH₂CH₃ | H | H | H |
| 1.101 | CH₂CH₃ | CH₃ | H | H |
| 1.102 | CH₂CH₃ | H | CH₃ | H |
| 1.103 | CH₂CH₃ | H | H | CH₃ |
| 1.104 | CH₂CH₃ | CH₃ | CH₃ | H |
| 1.105 | CH₂CH₃ | CH₃ | H | CH₃ |
| 1.106 | CH₂CH₃ | CH₃ | CH₃ | CH₃ |
| 1.107 | CH₂CH₃ | Cl | H | H |
| 1.108 | CH₂CH₃ | Cl | H | CH₃ |
| 1.109 | CH₂CH₃ | Cl | H | OCH₃ |
| 1.110 | CH₂CH₃ | H | Cl | H |
| 1.111 | CH₂CH₃ | H | H | Cl |
| 1.112 | CH₂CH₃ | CH₃ | Cl | H |
| 1.113 | CH₂CH₃ | CH₃ | H | Cl |
| 1.114 | CH₂CH₃ | H | Cl | CH₃ |
| 1.115 | CH₂CH₃ | CH₃ | Cl | CH₃ |
| 1.116 | CH₂CH₃ | Br | H | H |
| 1.117 | CH₂CH₃ | Br | H | CH₃ |
| 1.118 | CH₂CH₃ | Br | H | OCH₃ |
| 1.119 | CH₂CH₃ | H | Br | H |
| 1.120 | CH₂CH₃ | H | H | Br |
| 1.121 | CH₂CH₃ | CH₃ | Br | H |
| 1.122 | CH₂CH₃ | CH₃ | H | Br |
| 1.123 | CH₂CH₃ | H | Br | CH₃ |
| 1.124 | CH₂CH₃ | CH₃ | Br | CH₃ |
| 1.125 | CH₂CH₃ | CH₃O | H | H |
| 1.126 | CH₂CH₃ | CH₃O | H | CH₃ |
| 1.127 | CH₂CH₃ | CH₃O | H | Cl |
| 1.128 | CH₂CH₃ | CH₃O | H | Br |
| 1.129 | CH₂CH₃ | CH₃CH₂O | H | H |
| 1.130 | CH₂CH₃ | CH₃CH₂O | H | CH₃ |
| 1.131 | CH₂CH₃ | CH₃CH₂O | H | Cl |
| 1.132 | CH₂CH₃ | CH₃CH₂O | H | Br |
| 1.133 | CH₂CH₃ | H | CH₃O | H |
| 1.134 | CH₂CH₃ | H | H | CH₃O |
| 1.135 | CH₂CH₃ | CH₃ | CH₃O | H |
| 1.136 | CH₂CH₃ | CH₃ | H | CH₃O |
| 1.137 | CH₂CH₃ | H | CH₃O | CH₃ |
| 1.138 | CH₂CH₃ | CH₃ | CH₃O | CH₃ |
| 1.139 | CH₂CH₃ | —CH═CH₂ | H | CH₃ |
| 1.140 | CH₂CH₃ | CH₃ | H | —CH═CH₂ |
| 1.141 | CH₂CH₃ | —C•CH | H | CH₃ |
| 1.142 | CH₂CH₃ | CH₃ | H | —C•CH |
| 1.143 | CH₂CH₃ | —CH═CH₂ | H | —CH═CH₂ |
| 1.144 | CH₂CH₃ | CH₂CH₃ | H | CH₃ |
| 1.145 | CH₂CH₃ | phenyl | H | CH₃ |
| 1.146 | CH₂CH₃ | 2-fluorophenyl | H | CH₃ |
| 1.147 | CH₂CH₃ | 2-chlorophenyl | H | CH₃ |
| 1.148 | CH₂CH₃ | 2-trifluoromethylphenyl | H | CH₃ |
| 1.149 | CH₂CH₃ | 2-nitrophenyl | H | CH₃ |
| 1.150 | CH₂CH₃ | 2-methylphenyl | H | CH₃ |
| 1.151 | CH₂CH₃ | 2-methylsulfonylphenyl | H | CH₃ |
| 1.152 | CH₂CH₃ | 2-cyanophenyl | H | CH₃ |
| 1.153 | CH₂CH₃ | 3-fluorophenyl | H | CH₃ |
| 1.154 | CH₂CH₃ | 3-chlorophenyl | H | CH₃ |
| 1.155 | CH₂CH₃ | 3-trifluoromethylphenyl | H | CH₃ |
| 1.156 | CH₂CH₃ | 3-nitrophenyl | H | CH₃ |
| 1.157 | CH₂CH₃ | 3-methylphenyl | H | CH₃ |
| 1.158 | CH₂CH₃ | 3-methylsulfonylphenyl | H | CH₃ |
| 1.159 | CH₂CH₃ | 3-cyanophenyl | H | CH₃ |
| 1.160 | CH₂CH₃ | 4-fluorophenyl | H | CH₃ |
| 1.161 | CH₂CH₃ | 4-chlorophenyl | H | CH₃ |
| 1.162 | CH₂CH₃ | 4-bromophenyl | H | CH₃ |
| 1.163 | CH₂CH₃ | 4- | H | CH₃ |
| | | difluoromethoxyphenyl | | |
| 1.164 | CH₂CH₃ | 2-fluoro-4-chlorophenyl | H | CH₃ |
| 1.165 | CH₂CH₃ | 2-chloro-4-chlorophenyl | H | CH₃ |
| 1.166 | CH₂CH₃ | 2-methyl-4- | H | CH₃ |
| | | chlorophenyl | | |
| 1.167 | CH₂CH₃ | 4-trifluoromethylphenyl | H | CH₃ |
| 1.168 | CH₂CH₃ | 4-nitrophenyl | H | CH₃ |
| 1.169 | CH₂CH₃ | 4-methylphenyl | H | CH₃ |
| 1.170 | CH₂CH₃ | 4-methylsulfonylphenyl | H | CH₃ |
| 1.171 | CH₂CH₃ | 4-cyanophenyl | H | CH₃ |
| 1.172 | CH₂CH₃ | H | phenyl | H |
| 1.173 | CH₂CH₃ | H | 2-fluorophenyl | H |
| 1.174 | CH₂CH₃ | H | 2-chlorophenyl | H |
| 1.175 | CH₂CH₃ | H | 2-trifluoromethylphenyl | H |
| 1.176 | CH₂CH₃ | H | 2-nitrophenyl | H |
| 1.177 | CH₂CH₃ | H | 2-methylphenyl | H |
| 1.178 | CH₂CH₃ | H | 2-methylsulfonylphenyl | H |
| 1.179 | CH₂CH₃ | H | 2-cyanophenyl | H |
| 1.180 | CH₂CH₃ | H | 3-fluorophenyl | H |
| 1.181 | CH₂CH₃ | H | 3-chlorophenyl | H |
| 1.182 | CH₂CH₃ | H | 3-trifluoromethylphenyl | H |
| 1.183 | CH₂CH₃ | H | 3-nitrophenyl | H |
| 1.184 | CH₂CH₃ | H | 3-methylphenyl | H |
| 1.185 | CH₂CH₃ | H | 3-methylsulfonylphenyl | H |
| 1.186 | CH₂CH₃ | H | 3-cyanophenyl | H |
| 1.187 | CH₂CH₃ | H | 4-fluorophenyl | H |
| 1.188 | CH₂CH₃ | H | 4-chlorophenyl | H |
| 1.189 | CH₂CH₃ | H | 4-bromophenyl | H |
| 1.190 | CH₂CH₃ | H | 4- | H |
| | | | difluoromethoxyphenyl | |
| 1.191 | CH₂CH₃ | H | 2-fluoro-4-chlorophenyl | H |
| 1.192 | CH₂CH₃ | H | 2-chloro-4-chlorophenyl | H |
| 1.193 | CH₂CH₃ | H | 2-methyl-4- | H |
| | | | chlorophenyl | |
| 1.194 | CH₂CH₃ | H | 4-trifluoromethylphenyl | H |
| 1.195 | CH₂CH₃ | H | 4-nitrophenyl | H |
| 1.196 | CH₂CH₃ | H | 4-methylphenyl | H |
| 1.197 | CH₂CH₃ | H | 4-methylsulfonylphenyl | H |
| 1.198 | CH₂CH₃ | H | 4-cyanophenyl | H |
| 1.199 | CH₂CH₃ | CH₃ | H | CH₂CH₃ |
| 1.200 | CH₂CH₃ | CH₂CH₃ | H | CH₂CH₃ |
| 1.201 | CH₂CH₃ | Cl | H | CH₂CH₃ |
| 1.202 | CH₂CH₃ | Br | H | CH₂CH₃ |
| 1.203 | CH₂CH₃ | NO₂ | H | CH₂CH₃ |
| 1.204 | CH₂CH₃ | CH₃O | H | CH₂CH₃ |
| 1.205 | CH₂CH₃ | CH₃S | H | CH₂CH₃ |
| 1.206 | CH₂CH₃ | CH₃SO₂ | H | CH₂CH₃ |
| 1.207 | CH₂CH₃ | CH₂═CH | H | CH₂CH₃ |
| 1.208 | CH₂CH₃ | —C•CH | H | CH₂CH₃ |
| 1.209 | CH₂CH₃ | phenyl | H | CH₂CH₃ |
| 1.210 | CH₂CH₃ | 2-fluorophenyl | H | CH₂CH₃ |
| 1.211 | CH₂CH₃ | 2-chlorophenyl | H | CH₂CH₃ |
| 1.212 | CH₂CH₃ | 2-trifluoromethylphenyl | H | CH₂CH₃ |
| 1.213 | CH₂CH₃ | 2-nitrophenyl | H | CH₂CH₃ |
| 1.214 | CH₂CH₃ | 2-methylphenyl | H | CH₂CH₃ |
| 1.215 | CH₂CH₃ | 2-methylsulfonylphenyl | H | CH₂CH₃ |
| 1.216 | CH₂CH₃ | 2-cyanophenyl | H | CH₂CH₃ |
| 1.217 | CH₂CH₃ | 3-fluorophenyl | H | CH₂CH₃ |
| 1.218 | CH₂CH₃ | 3-chlorophenyl | H | CH₂CH₃ |
| 1.219 | CH₂CH₃ | 3-trifluoromethylphenyl | H | CH₂CH₃ |
| 1.220 | CH₂CH₃ | 3-nitrophenyl | H | CH₂CH₃ |
| 1.221 | CH₂CH₃ | 3-methylphenyl | H | CH₂CH₃ |
| 1.222 | CH₂CH₃ | 3-methylsulfonylphenyl | H | CH₂CH₃ |
| 1.223 | CH₂CH₃ | 3-cyanophenyl | H | CH₂CH₃ |
| 1.224 | CH₂CH₃ | 4-fluorophenyl | H | CH₂CH₃ |
| 1.225 | CH₂CH₃ | 4-chlorophenyl | H | CH₂CH₃ |
| 1.226 | CH₂CH₃ | 4-bromophenyl | H | CH₂CH₃ |
| 1.227 | CH₂CH₃ | 4- | H | CH₂CH₃ |
| | | difluoromethoxyphenyl | | |
| 1.228 | CH₂CH₃ | 2-fluoro-4-chlorophenyl | H | CH₂CH₃ |
| 1.229 | CH₂CH₃ | 2-chloro-4-chlorophenyl | H | CH₂CH₃ |
| 1.230 | CH₂CH₃ | 2-methyl-4- | H | CH₂CH₃ |
| | | chlorophenyl | | |
| 1.231 | CH₂CH₃ | 4-trifluoromethylphenyl | H | CH₂CH₃ |
| 1.232 | CH₂CH₃ | 4-nitrophenyl | H | CH₂CH₃ |
| 1.233 | CH₂CH₃ | 4-methylphenyl | H | CH₂CH₃ |
| 1.234 | CH₂CH₃ | 4-methylsulfonylphenyl | H | CH₂CH₃ |
| 1.235 | CH₂CH₃ | 4-cyanophenyl | H | CH₂CH₃ |
| 1.236 | OCH₃ | H | phenyl | H |
| 1.237 | OCH₃ | H | 2-fluorophenyl | H |
| 1.238 | OCH₃ | H | 2-chlorophenyl | H |
| 1.239 | OCH₃ | H | 2-trifluoromethylphenyl | H |
| 1.240 | OCH₃ | H | 2-nitrophenyl | H |
| 1.241 | OCH₃ | H | 2-methylphenyl | H |
| 1.242 | OCH₃ | H | 2-methylsulfonylphenyl | H |
| 1.243 | OCH₃ | H | 2-cyanophenyl | H |
| 1.244 | OCH₃ | H | 3-fluorophenyl | H |
| 1.245 | OCH₃ | H | 3-chlorophenyl | H |
| 1.246 | OCH₃ | H | 3-trifluoromethylphenyl | H |
| 1.247 | OCH₃ | H | 3-nitrophenyl | H |
| 1.248 | OCH₃ | H | 3-methylphenyl | H |
| 1.249 | OCH₃ | H | 3-methylsulfonylphenyl | H |
| 1.250 | OCH₃ | H | 3-cyanophenyl | H |
| 1.251 | OCH₃ | H | 4-fluorophenyl | H |
| 1.252 | OCH₃ | H | 4-chlorophenyl | H |
| 1.253 | OCH₃ | H | 4-bromophenyl | H |
| 1.254 | OCH₃ | H | 4- | H |
| | | | difluoromethoxyphenyl | |
| 1.255 | OCH₃ | H | 2-fluoro-4-chlorophenyl | H |
| 1.256 | OCH₃ | H | 2-chloro-4-chlorophenyl | H |
| 1.257 | OCH₃ | H | 2-methyl-4- | H |
| | | | chlorophenyl | |
| 1.258 | OCH₃ | H | 4-trifluoromethylphenyl | H |
| 1.259 | OCH₃ | H | 4-nitrophenyl | H |
| 1.260 | OCH₃ | H | 4-methylphenyl | H |
| 1.261 | OCH₃ | H | 4-methylsulfonylphenyl | H |
| 1.262 | OCH₃ | H | 4-cyanophenyl | H |

Table 2 covers 262 compounds of the following type

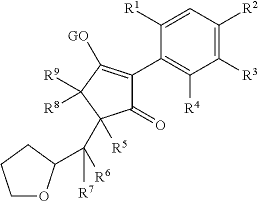

where G, R⁵, R⁶, R⁸ and R⁹ are hydrogen, R⁷ is methyl and R¹, R², R³ and R⁴ are as described in Table 1.

Table 3 covers 262 compounds of the following type

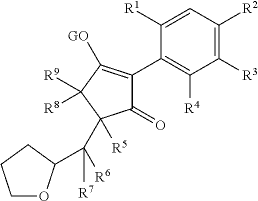

where G and R⁵, R⁸ and R⁹ are hydrogen, R⁶ and Ware methyl and R¹, R², R³ and R⁴ are as described in Table 1.

Table 4 covers 262 compounds of the following type:

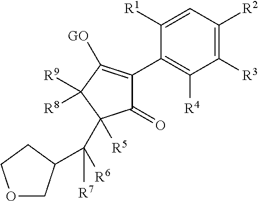

where G, R⁵, R⁶, R⁷, R⁸ and R⁹ are all hydrogen, and R¹, R², R³ and R⁴ are as described in Table 1.

Table 5 covers 262 compounds of the following type

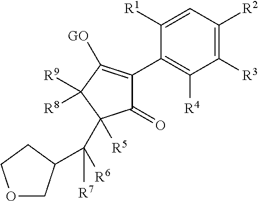

where G, R⁵, R⁶, R⁸ and R⁹ are hydrogen, R⁷ is methyl and R¹, R², R³ and R⁴ are as described in Table 1.

Table 6 covers 262 compounds of the following type

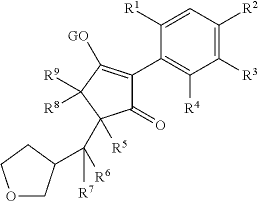

where G and R⁵, R⁸ and R⁹ are hydrogen, R⁶ and Ware methyl and R¹, R², R³ and R⁴ are as described in Table 1.

Table 7 covers 262 compounds of the following type:

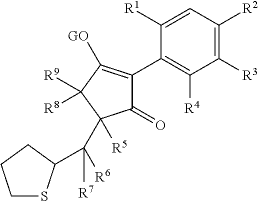

where G, R⁵, R⁶, R⁷, R⁸ and R⁹ are all hydrogen, and R¹, R², R³ and R⁴ are as described in Table 1.

Table 8 covers 262 compounds of the following type

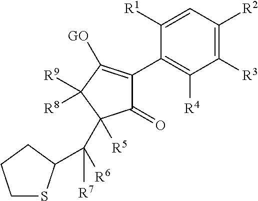

where G, R⁵, R⁶, R⁸ and R⁹ are hydrogen, R⁷ is methyl and R¹, R², R³ and R⁴ are as described in Table 1.

Table 9 covers 262 compounds of the following type

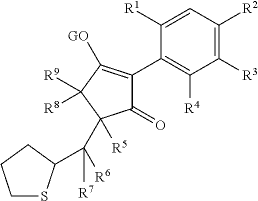

where G and R⁵, R⁸ and R⁹ are hydrogen, R⁶ and Ware methyl and R¹, R², R³ and R⁴ are as described in Table 1.

Table 10 covers 262 compounds of the following type:

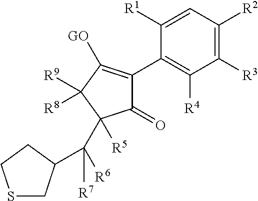

where G, R⁵, R⁶, R⁷, R⁸ and R⁹ are all hydrogen, and R¹, R², R³ and R⁴ are as described in Table 1.

Table 11 covers 262 compounds of the following type

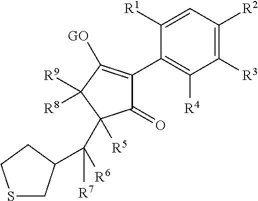

where G, R⁵, R⁶, R⁸ and R⁹ are hydrogen, R⁷ is methyl and R¹, R², R³ and R⁴ are as described in Table 1.

Table 12 covers 262 compounds of the following type

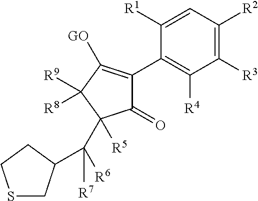

where G and R⁵, R⁸ and R⁹ are hydrogen, R⁶ and Ware methyl and R¹, R², R³ and R⁴ are as described in Table 1.

Table 13 covers 262 compounds of the following type:

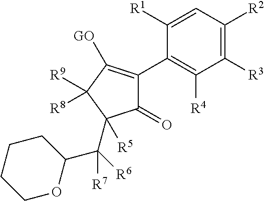

where G, R⁵, R⁶, R⁷, R⁸ and R⁹ are all hydrogen, and R¹, R², R³ and R⁴ are as described in Table 1.

Table 14 covers 262 compounds of the following type

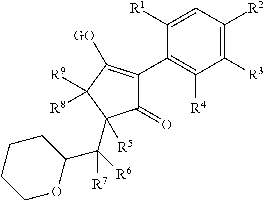

where G, R⁵, R⁶, R⁸ and R⁹ are hydrogen, R⁷ is methyl and R¹, R², R³ and R⁴ are as described in Table 1.

Table 15 covers 262 compounds of the following type

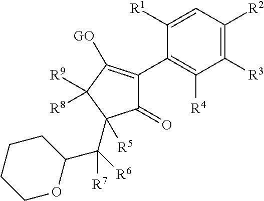

where G and R⁵, R⁸ and R⁹ are hydrogen, R⁶ and Ware methyl and R¹, R², R³ and R⁴ are as described in Table 1.

Table 16 covers 262 compounds of the following type:

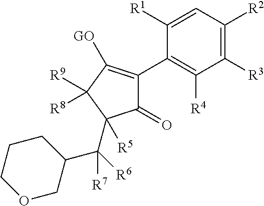

where G, R⁵, R⁶, R⁷, R⁸ and R⁹ are all hydrogen, and R¹, R², R³ and R⁴ are as described in Table 1.

Table 17 covers 262 compounds of the following type

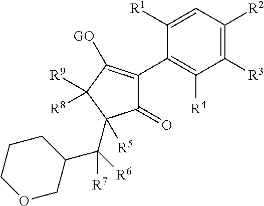

where G, R⁵, R⁶, R⁸ and R⁹ are hydrogen, R⁷ is methyl and R¹, R², R³ and R⁴ are as described in Table 1.

Table 18 covers 262 compounds of the following type

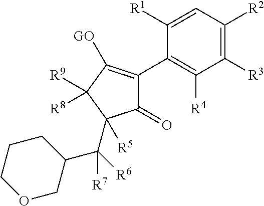

where G and R⁵, R⁸ and R⁹ are hydrogen, R⁶ and Ware methyl and R¹, R², R³ and R⁴ are as described in Table 1.

Table 19 covers 262 compounds of the following type:

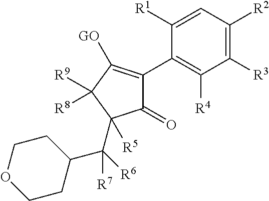

where G, R⁵, R⁶, R⁷, R⁸ and R⁹ are all hydrogen, and R¹, R², R³ and R⁴ are as described in Table 1.

Table 20 covers 262 compounds of the following type

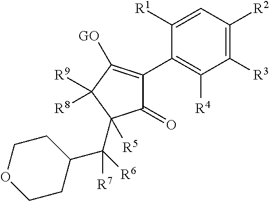

where G, R⁵, R⁶, R⁸ and R⁹ are hydrogen, R⁷ is methyl and R¹, R², R³ and R⁴ are as described in Table 1.

Table 21 covers 262 compounds of the following type

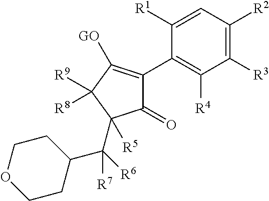

where G and R⁵, R⁸ and R⁹ are hydrogen, R⁶ and Ware methyl and R¹, R², R³ and R⁴ are as described in Table 1.

Table 22 covers 262 compounds of the following type:

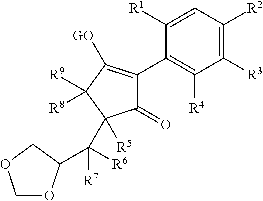

where G, R⁵, R⁶, R⁷, R⁸ and R⁹ are all hydrogen, and R¹, R², R³ and R⁴ are as described in Table 1.

Table 23 covers 262 compounds of the following type

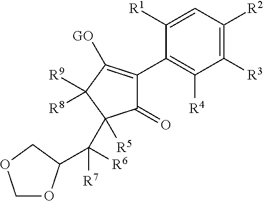

where G, R⁵, R⁶, R⁸ and R⁹ are hydrogen, R⁷ is methyl and R¹, R², R³ and R⁴ are as described in Table 1.

Table 24 covers 262 compounds of the following type

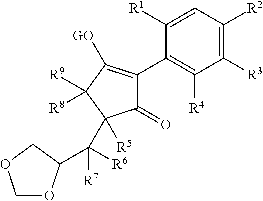

where G and R⁵, R⁸ and R⁹ are hydrogen, R⁶ and Ware methyl and R¹, R², R³ and R⁴ are as described in Table 1.

Table 25 covers 262 compounds of the following type:

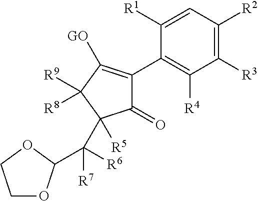

where G, R⁵, R⁶, R⁷, R⁸ and R⁹ are all hydrogen, and R¹, R², R³ and R⁴ are as described in Table 1.

Table 26 covers 262 compounds of the following type

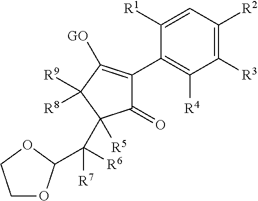

where G, R⁵, R⁶, R⁸ and R⁹ are hydrogen, R⁷ is methyl and R¹, R², R³ and R⁴ are as described in Table 1.

Table 27 covers 262 compounds of the following type

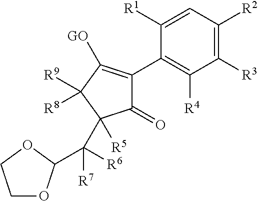

where G and R⁵, R⁸ and R⁹ are hydrogen, R⁶ and Ware methyl and R¹, R², R³ and R⁴ are as described in Table 1.

Table 28 covers 262 compounds of the following type:

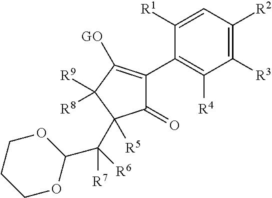

where G, R⁵, R⁶, R⁷, R⁸ and R⁹ are all hydrogen, and R¹, R², R³ and R⁴ are as described in Table 1.

Table 29 covers 262 compounds of the following type

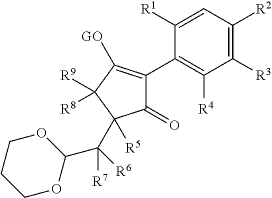

where G, R⁵, R⁶, R⁸ and R⁹ are hydrogen, R⁷ is methyl and R¹, R², R³ and R⁴ are as described in Table 1.

Table 30 covers 262 compounds of the following type

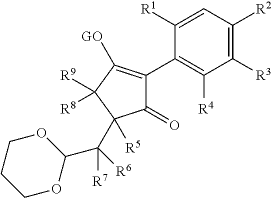

where G and R⁵, R⁸ and R⁹ are hydrogen, R⁶ and Ware methyl and R¹, R², R³ and R⁴ are as described in Table 1.

Table 31 covers 262 compounds of the following type:

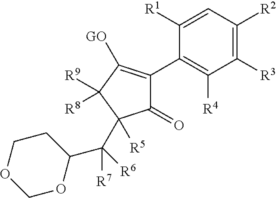

where G, R⁵, R⁶, R⁷, R⁸ and R⁹ are all hydrogen, and R¹, R², R³ and R⁴ are as described in Table 1.

Table 32 covers 262 compounds of the following type

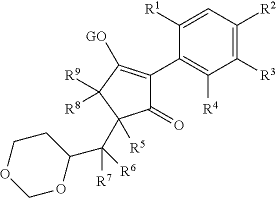

where G, R⁵, R⁶, R⁸ and R⁹ are hydrogen, R⁷ is methyl and R¹, R², R³ and R⁴ are as described in Table 1.

Table 33 covers 262 compounds of the following type

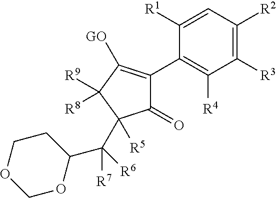

where G and R⁵, R⁸ and R⁹ are hydrogen, R⁶ and Ware methyl and R¹, R², R³ and R⁴ are as described in Table 1.

Table 34 covers 262 compounds of the following type:

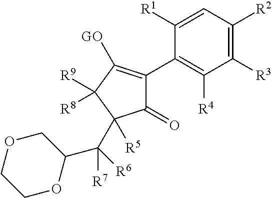

where G, R⁵, R⁶, R⁷, R⁸ and R⁹ are all hydrogen, and R¹, R², R³ and R⁴ are as described in Table 1.

Table 35 covers 262 compounds of the following type

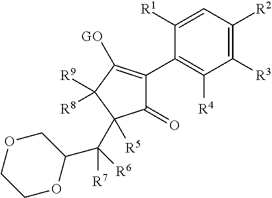

where G, R⁵, R⁶, R⁸ and R⁹ are hydrogen, R⁷ is methyl and R¹, R², R³ and R⁴ are as described in Table 1.

Table 36 covers 262 compounds of the following type

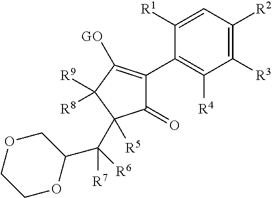

where G and R⁵, R⁸ and R⁹ are hydrogen, R⁶ and Ware methyl and R¹, R², R³ and R⁴ are as described in Table 1.

Table 37 covers 262 compounds of the following type:

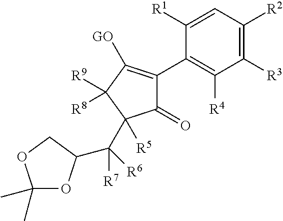

where G, R⁵, R⁶, R⁷, R⁸ and R⁹ are all hydrogen, and R¹, R², R³ and R⁴ are as described in Table 1.

Table 38 covers 262 compounds of the following type

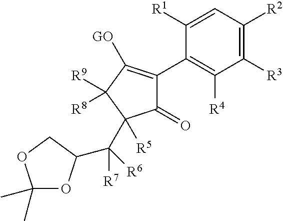

where G, R⁵, R⁶, R⁸ and R⁹ are hydrogen, R⁷ is methyl and R¹, R², R³ and R⁴ are as described in Table 1.

Table 39 covers 262 compounds of the following type

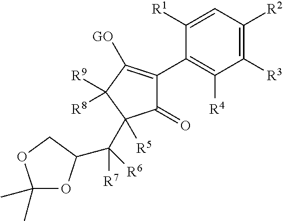

where G and R⁵, R⁸ and R⁹ are hydrogen, R⁶ and Ware methyl and R¹, R², R³ and R⁴ are as described in Table 1.

Table 40 covers 262 compounds of the following type:

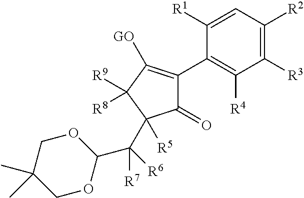

where G, R⁵, R⁶, R⁷, R⁸ and R⁹ are all hydrogen, and R¹, R², R³ and R⁴ are as described in Table 1.

Table 41 covers 262 compounds of the following type

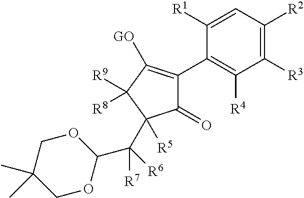

where G, R⁵, R⁶, R⁸ and R⁹ are hydrogen, R⁷ is methyl and R¹, R², R³ and R⁴ are as described in Table 1.

Table 42 covers 262 compounds of the following type

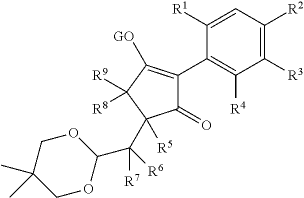

where G and R⁵, R⁸ and R⁹ are hydrogen, R⁶ and Ware methyl and R¹, R², R³ and R⁴ are as described in Table 1.

Table 43 covers 262 compounds of the following type:

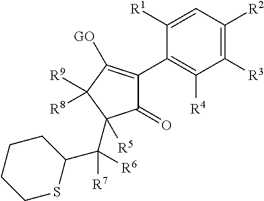

where G, R⁵, R⁶, R⁷, R⁸ and R⁹ are all hydrogen, and R¹, R², R³ and R⁴ are as described in Table 1.

Table 44 covers 262 compounds of the following type

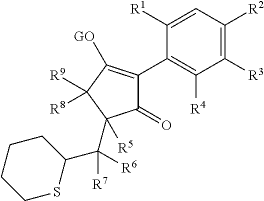

where G, R⁵, R⁶, R⁸ and R⁹ are hydrogen, R⁷ is methyl and R¹, R², R³ and R⁴ are as described in Table 1.

Table 45 covers 262 compounds of the following type

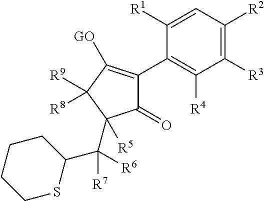

where G and R⁵, R⁸ and R⁹ are hydrogen, R⁶ and Ware methyl and R¹, R², R³ and R⁴ are as described in Table 1.

Table 46 covers 262 compounds of the following type:

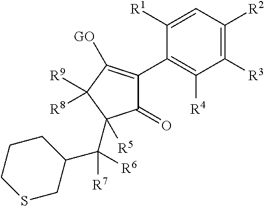

where G, R⁵, R⁶, R⁷, R⁸ and R⁹ are all hydrogen, and R¹, R², R³ and R⁴ are as described in Table 1.

Table 47 covers 262 compounds of the following type

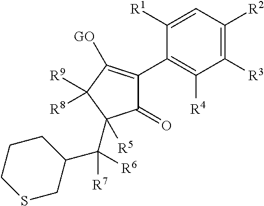

where G, R⁵, R⁶, R⁸ and R⁹ are hydrogen, R⁷ is methyl and R¹, R², R³ and R⁴ are as described in Table 1.

Table 48 covers 262 compounds of the following type

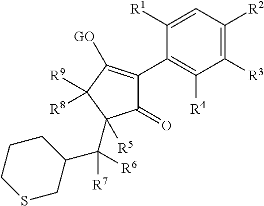

where G and R⁵, R⁸ and R⁹ are hydrogen, R⁶ and Ware methyl and R¹, R², R³ and R⁴ are as described in Table 1.

Table 49 covers 262 compounds of the following type:

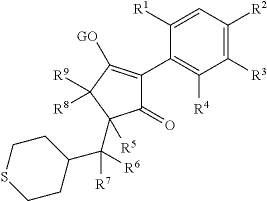

where G, R⁵, R⁶, R⁷, R⁸ and R⁹ are all hydrogen, and R¹, R², R³ and R⁴ are as described in Table 1.

Table 50 covers 262 compounds of the following type

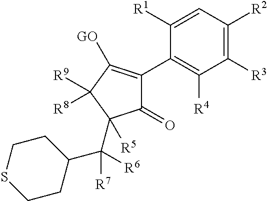

where G, R⁵, R⁶, R⁸ and R⁹ are hydrogen, R⁷ is methyl and R¹, R², R³ and R⁴ are as described in Table 1.

Table 51 covers 262 compounds of the following type

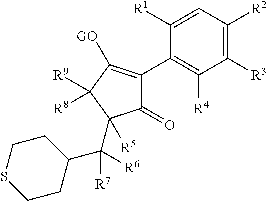

where G and R⁵, R⁸ and R⁹ are hydrogen, R⁶ and Ware methyl and R¹, R², R³ and R⁴ are as described in Table 1.

Table 52 covers 262 compounds of the following type:

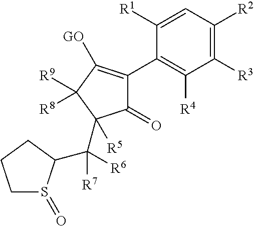

where G, R⁵, R⁶, R⁷, R⁸ and R⁹ are all hydrogen, and R¹, R², R³ and R⁴ are as described in Table 1.

Table 53 covers 262 compounds of the following type

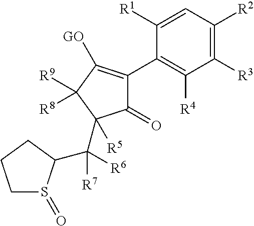

where G, R⁵, R⁶, R⁸ and R⁹ are hydrogen, R⁷ is methyl and R¹, R², R³ and R⁴ are as described in Table 1.

Table 54 covers 262 compounds of the following type

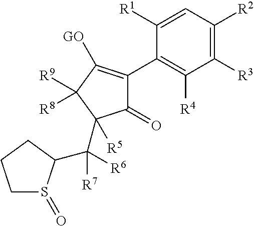

where G and R⁵, R⁸ and R⁹ are hydrogen, R⁶ and Ware methyl and R¹, R², R³ and R⁴ are as described in Table 1.

Table 55 covers 262 compounds of the following type:

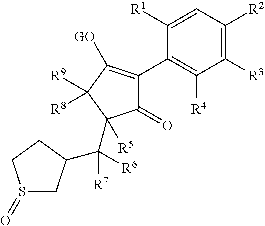

where G, R⁵, R⁶, R⁷, R⁸ and R⁹ are all hydrogen, and R¹, R², R³ and R⁴ are as described in Table 1.

Table 56 covers 262 compounds of the following type

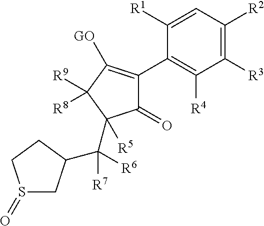

where G, R⁵, R⁶, R⁸ and R⁹ are hydrogen, R⁷ is methyl and R¹, R², R³ and R⁴ are as described in Table 1.

Table 57 covers 262 compounds of the following type

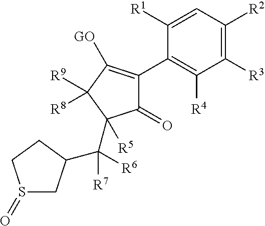

where G and R⁵, R⁸ and R⁹ are hydrogen, R⁶ and Ware methyl and R¹, R², R³ and R⁴ are as described in Table 1.

Table 58 covers 262 compounds of the following type:

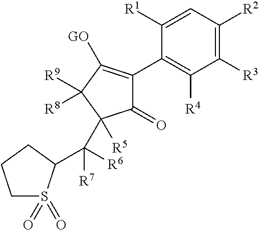

where G, R⁵, R⁶, R⁷, R⁸ and R⁹ are all hydrogen, and R¹, R², R³ and R⁴ are as described in Table 1.

Table 59 covers 262 compounds of the following type

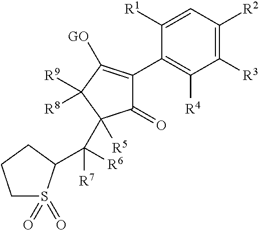

where G, R⁵, R⁶, R⁸ and R⁹ are hydrogen, R⁷ is methyl and R¹, R², R³ and R⁴ are as described in Table 1.

Table 60 covers 262 compounds of the following type

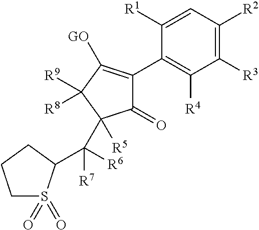

where G and R⁵, R⁸ and R⁹ are hydrogen, R⁶ and Ware methyl and R¹, R², R³ and R⁴ are as described in Table 1.

Table 61 covers 262 compounds of the following type:

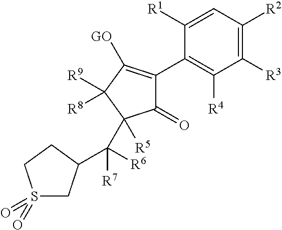

where G, R⁵, R⁶, R⁷, R⁸ and R⁹ are all hydrogen, and R¹, R², R³ and R⁴ are as described in Table 1.

Table 62 covers 262 compounds of the following type

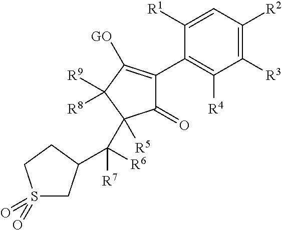

where G, R⁵, R⁶, R⁸ and R⁹ are hydrogen, R⁷ is methyl and R¹, R², R³ and R⁴ are as described in Table 1.

Table 63 covers 262 compounds of the following type

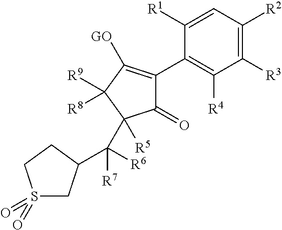

where G and R⁵, R⁸ and R⁹ are hydrogen, R⁶ and Ware methyl and R¹, R², R³ and R⁴ are as described in Table 1.

Table 64 covers 262 compounds of the following type:

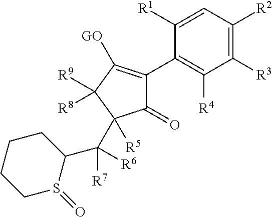

where G, R⁵, R⁶, R⁷, R⁸ and R⁹ are all hydrogen, and R¹, R², R³ and R⁴ are as described in Table 1.

Table 65 covers 262 compounds of the following type

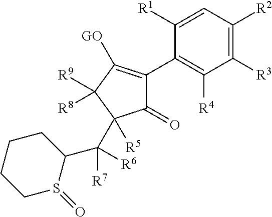

where G, R⁵, R⁶, R⁸ and R⁹ are hydrogen, R⁷ is methyl and R¹, R², R³ and R⁴ are as described in Table 1.

Table 66 covers 262 compounds of the following type

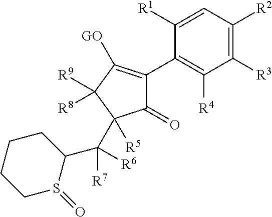

where G and R⁵, R⁸ and R⁹ are hydrogen, R⁶ and Ware methyl and R¹, R², R³ and R⁴ are as described in Table 1.

Table 67 covers 262 compounds of the following type:

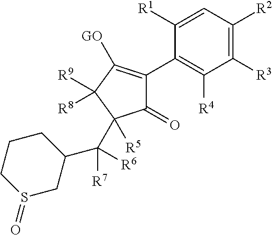

where G, R⁵, R⁶, R⁷, R⁸ and R⁹ are all hydrogen, and R¹, R², R³ and R⁴ are as described in Table 1.

Table 68 covers 262 compounds of the following type

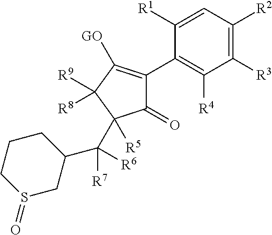

where G, R⁵, R⁶, R⁸ and R⁹ are hydrogen, R⁷ is methyl and R¹, R², R³ and R⁴ are as described in Table 1.

Table 69 covers 262 compounds of the following type

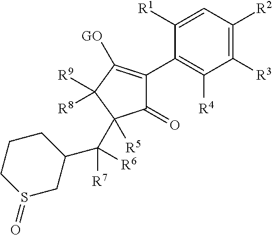

where G and R⁵, R⁸ and R⁹ are hydrogen, R⁶ and Ware methyl and R¹, R², R³ and R⁴ are as described in Table 1.

Table 70 covers 262 compounds of the following type:

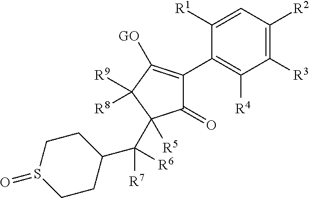

where G, R⁵, R⁶, R⁷, R⁸ and R⁹ are all hydrogen, and R¹, R², R³ and R⁴ are as described in Table 1.

Table 71 covers 262 compounds of the following type

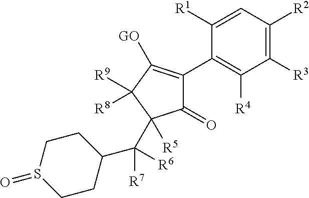

where G, R⁵, R⁶, R⁸ and R⁹ are hydrogen, R⁷ is methyl and R¹, R², R³ and R⁴ are as described in Table 1.

Table 72 covers 262 compounds of the following type

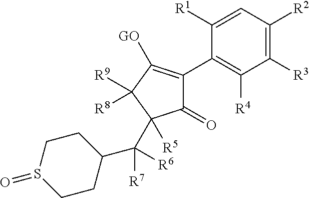

where G and R⁵, R⁸ and R⁹ are hydrogen, R⁶ and Ware methyl and R¹, R², R³ and R⁴ are as described in Table 1.

Table 73 covers 262 compounds of the following type:

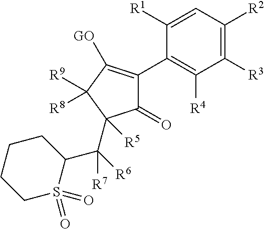

where G, R⁵, R⁶, R⁷, R⁸ and R⁹ are all hydrogen, and R¹, R², R³ and R⁴ are as described in Table 1.

Table 74 covers 262 compounds of the following type

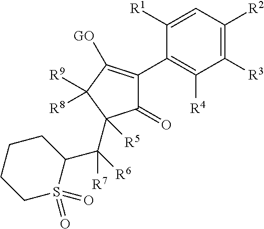

where G, R⁵, R⁶, R⁸ and R⁹ are hydrogen, R⁷ is methyl and R¹, R², R³ and R⁴ are as described in Table 1.

Table 75 covers 262 compounds of the following type

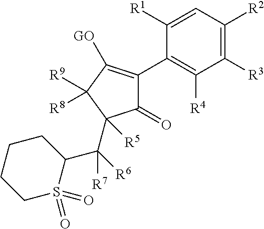

where G and R⁵, R⁸ and R⁹ are hydrogen, R⁶ and Ware methyl and R¹, R², R³ and R⁴ are as described in Table 1.

Table 76 covers 262 compounds of the following type:

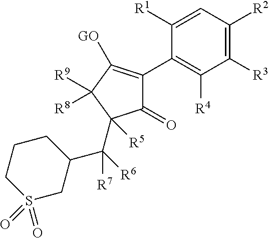

where G, R⁵, R⁶, R⁷, R⁸ and R⁹ are all hydrogen, and R¹, R², R³ and R⁴ are as described in Table 1.

Table 77 covers 262 compounds of the following type

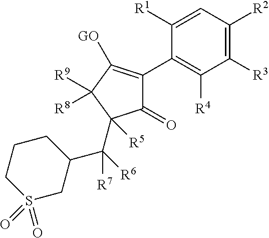

where G, R⁵, R⁶, R⁸ and R⁹ are hydrogen, R⁷ is methyl and R¹, R², R³ and R⁴ are as described in Table 1.

Table 78 covers 262 compounds of the following type

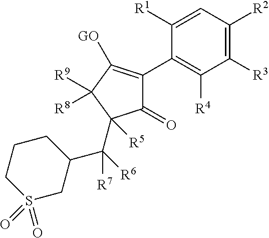

where G and R⁵, R⁸ and R⁹ are hydrogen, R⁶ and Ware methyl and R¹, R², R³ and R⁴ are as described in Table 1.

Table 79 covers 262 compounds of the following type:

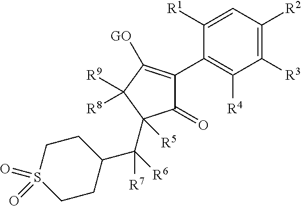

where G, R⁵, R⁶, R⁷, R⁸ and R⁹ are all hydrogen, and R¹, R², R³ and R⁴ are as described in Table 1.

to Table 80 covers 262 compounds of the following type

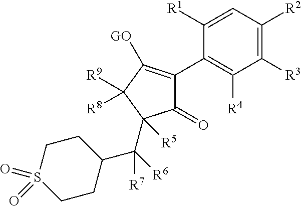

where G, R⁵, R⁶, R⁸ and R⁹ are hydrogen, R⁷ is methyl and R¹, R², R³ and R⁴ are as described in Table 1.

Table 81 covers 262 compounds of the following type

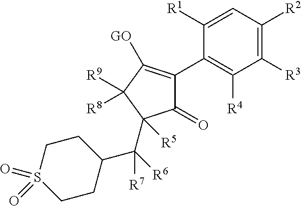

where G and R⁵, R⁸ and R⁹ are hydrogen, R⁶ and Ware methyl and R¹, R², R³ and R⁴ are as described in Table 1.

Table 82 covers 262 compounds of the following type:

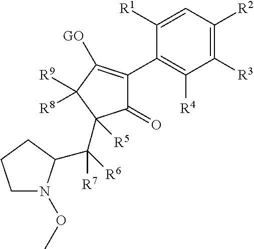

where G, R⁵, R⁶, R⁷, R⁸ and R⁹ are all hydrogen, and R¹, R², R³ and R⁴ are as described in Table 1.

Table 83 covers 262 compounds of the following type

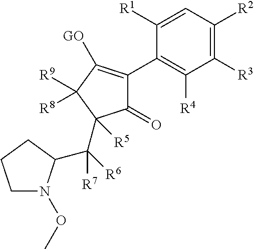

where G, R⁵, R⁶, R⁸ and R⁹ are hydrogen, R⁷ is methyl and R¹, R², R³ and R⁴ are as described in Table 1.

Table 84 covers 262 compounds of the following type

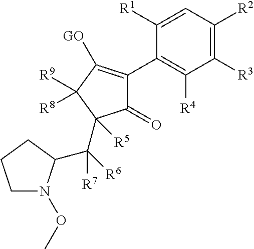

where G and R⁵, R⁸ and R⁹ are hydrogen, R⁶ and Ware methyl and R¹, R², R³ and R⁴ are as described in Table 1.

Table 85 covers 262 compounds of the following type:

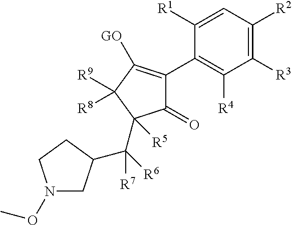

where G, R⁵, R⁶, R⁷, R⁸ and R⁹ are all hydrogen, and R¹, R², R³ and R⁴ are as described in Table 1.

Table 86 covers 262 compounds of the following type

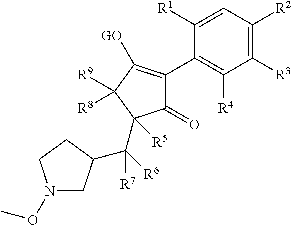

where G, R⁵, R⁶, R⁸ and R⁹ are hydrogen, R⁷ is methyl and R¹, R², R³ and R⁴ are as described in Table 1.

Table 87 covers 262 compounds of the following type

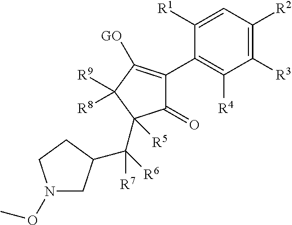

where G and R⁵, R⁸ and R⁹ are hydrogen, R⁶ and Ware methyl and R¹, R², R³ and R⁴ are as described in Table 1.

Table 88 covers 262 compounds of the following type:

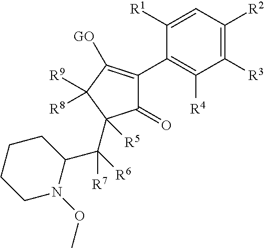

where G, R⁵, R⁶, R⁷, R⁸ and R⁹ are all hydrogen, and R¹, R², R³ and R⁴ are as described in Table 1.

Table 89 covers 262 compounds of the following type

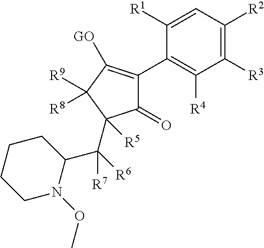

where G, R⁵, R⁶, R⁸ and R⁹ are hydrogen, R⁷ is methyl and R¹, R², R³ and R⁴ are as described in Table 1.

Table 90 covers 262 compounds of the following type

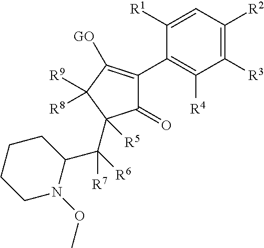

where G and R⁵, R⁸ and R⁹ are hydrogen, R⁶ and Ware methyl and R¹, R², R³ and R⁴ are as described in Table 1.

Table 91 covers 262 compounds of the following type:

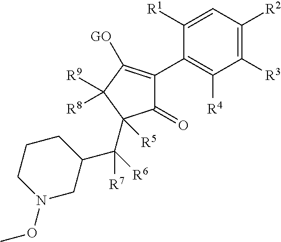

where G, R⁵, R⁶, R⁷, R⁸ and R⁹ are all hydrogen, and R¹, R², R³ and R⁴ are as described in Table 1.

Table 92 covers 262 compounds of the following type

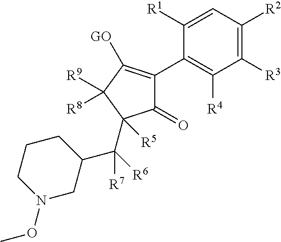

where G, R⁵, R⁶, R⁸ and R⁹ are hydrogen, R⁷ is methyl and R¹, R², R³ and R⁴ are as described in Table 1.

Table 93 covers 262 compounds of the following type

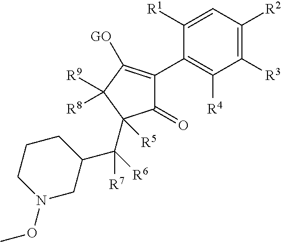

where G and R⁵, R⁸ and R⁹ are hydrogen, R⁶ and Ware methyl and R¹, R², R³ and R⁴ are as described in Table 1.

Table 94 covers 262 compounds of the following type:

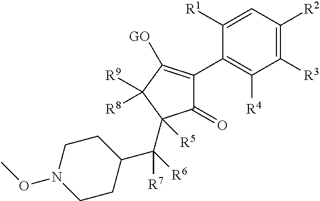

where G, R⁵, R⁶, R⁷, R⁸ and R⁹ are all hydrogen, and R¹, R², R³ and R⁴ are as described in Table 1.

Table 95 covers 262 compounds of the following type

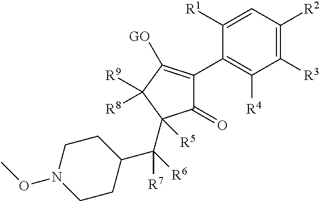

where G, R⁵, R⁶, R⁸ and R⁹ are hydrogen, R⁷ is methyl and R¹, R², R³ and R⁴ are as described in Table 1.

Table 96 covers 262 compounds of the following type

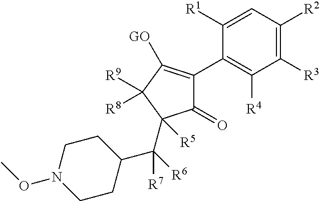

where G and R⁵, R⁸ and R⁹ are hydrogen, R⁶ and Ware methyl and R¹, R², R³ and R⁴ are as described in Table 1.

Table 97 covers 262 compounds of the following type:

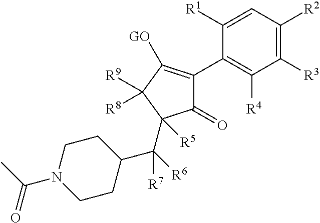

where G, R⁵, R⁶, R⁷, R⁸ and R⁹ are all hydrogen, and R¹, R², R³ and R⁴ are as described in Table 1.

Table 98 covers 262 compounds of the following type

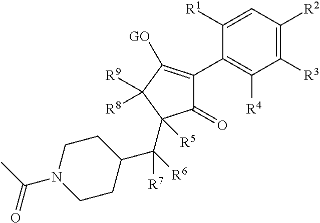

where G, R⁵, R⁶, R⁸ and R⁹ are hydrogen, R⁷ is methyl and R¹, R², R³ and R⁴ are as described in Table 1.

Table 99 covers 262 compounds of the following type

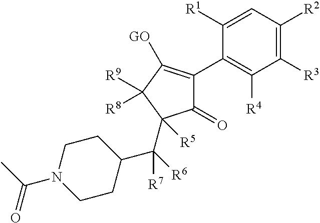

where G and R⁵, R⁸ and R⁹ are hydrogen, R⁶ and Ware methyl and R¹, R², R³ and R⁴ are as described in Table 1.

Table 100 covers 262 compounds of the following type:

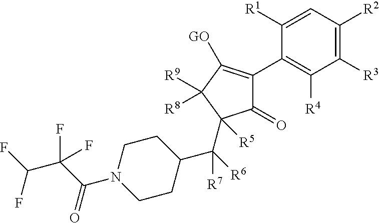

where G, R⁵, R⁶, R⁷, R⁸ and R⁹ are all hydrogen, and R¹, R², R³ and R⁴ are as described in Table 1.

Table 101 covers 262 compounds of the following type

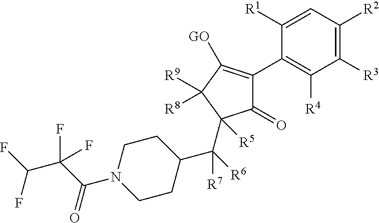

where G, R⁵, R⁶, R⁸ and R⁹ are hydrogen, R⁷ is methyl and R¹, R², R³ and R⁴ are as described in Table 1.

Table 102 covers 262 compounds of the following type

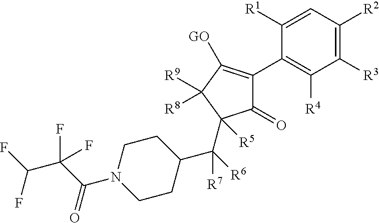

where G and R⁵, R⁸ and R⁹ are hydrogen, R⁶ and Ware methyl and R¹, R², R³ and R⁴ are as described in Table 1.

### Biological Examples

These examples illustrate the pesticidal/insecticidal properties of compounds of formula I.

### Example B1

### Activity Against Myzus persicae (Green Peach Aphid)

(mixed population, feeding/residual contact activity, preventive)

Sunflower leaf discs are placed on agar in a 24-well microtiter plate and sprayed with test solutions. After drying, the leaf discs are infested with an aphid population of mixed ages. After an incubation period of 6 days, samples are checked for mortality and special effects (e.g. phytotoxicity).

In this test, compounds listed in the tables above show good activity. In particular compounds T5, T8, T14, T15, T16, T17, T22, T23, T24, T29, T37, T45, T52, T59, T61, T62, T63, T68, T95, T98, T132, T138, T150, T169, T171, P7, P14, P16, P17, P44, P50, P57, P66 and P71 show an activity of over 80% at a concentration of 400 ppm.

### Example B2

### Activity Against Myzus persicae (Green Peach Aphid)

(mixed population, systemic/feeding activity, curative)

Roots of pea seedlings, infested with an aphid population of mixed ages, are placed directly in the test solutions. 6 days after introduction, samples are checked for mortality and special effects on the plant.

In this test, compounds listed in the tables above show good activity. For example compound T22 show an activity of over 80% at a concentration of 400 ppm.

### Example B3

### Activity Against Thrips tabaci (Onion Thrips)

(mixed population, feeding/residual contact activity, preventive)

Sunflower leaf discs are placed on agar in a 24-well microtiter plate and sprayed with test solutions. After drying, the leaf discs are infested with a thrips population of mixed ages. After an incubation period of 6 days, samples are checked for mortality and special effects (e.g. phytotoxicity).

In this test, compounds listed in the tables above show good activity. In particular compounds T4, T5, T8, T14, T16, T17, T23, T52, P7, P13, P15, P17, P18, P20, P23, P24, P26, P27, P30, P43, P44, P50, P55 and P56 show an activity of over 80% at a concentration of 400 ppm.

### Example B4

### Activity Against Tetranychus urticae (Two-Spotted Spider Mite)

(mixed population, feeding/residual contact activity, preventive)

Bean leaf discs on agar in 24-well microtiter plates are sprayed with test solutions. After drying, the leaf discs are infested with mite populations of mixed ages. 8 days later, discs are checked for egg mortality, larval mortality, and adult mortality.

In this test, compounds listed in the tables above show good activity. In particular compounds T6, T8, T14, T17, T21, T22, T30, T31, T34, T35, T37, T39, T40, T41, T43, T44, T45, T48, T50, T52, T54, T55, T58, T60, T61, T62, T63, T66, T67, T68, T72, T95, T98, T112, T128, T130, T131, T132, T134, T135, T136, T137, T138, T139, T140, T142, T158, T161, T162, T163, T164, T170, T171, T173, T175, P7, P17, P18, P23, P28, P34, P37, P44, P49, P51, P55, P56, P57, P58, P59, P62, P63, P65 and P71 show an activity of over 80% at a concentration of 400 ppm.

### Example B5

### Activity Against Plutella xylostella (Diamond Back Moth)

(larvicide, feeding/residual contact activity, preventive)

24-well microtiter plate (MTP) with artificial diet is treated with test solutions by pipetting. After drying, the MTP's are infested with larvae (L2)(10-15 per well). After an incubation period of 5 days, samples are checked for larval mortality, antifeedant and growth regulation.

In this test, compounds listed in the tables above show good activity. In particular compounds T4, T5, T8, T14, T16, T17, T31, T33, T62, T121, T127, P5, P7, P20, P37 and P44 show an activity of over 80% at a concentration of 400 ppm.

### Example B6

### Activity Against Diabrotica balteata (Corn Root Worm)

(larvicide, feeding/residual contact activity, preventive)

24-well microtiter plate (MTP) with artificial diet is treated with test solutions by pipetting. After drying, the MTP's are infested with larvae (L2)(6-10 per well). After an incubation period of 5 days, samples are checked for larval mortality, antifeedant and growth regulation.

In this test, compounds listed in the tables above show good activity. In particular compounds T17, T22, T39, T54, T160, P44 and P71 show an activity of over 80% at a concentration of 400 ppm.

### Example B7

### Activity Against Myzus persicae (Green Peach Aphid)

(mixed population, feeding activity)

Test compounds are applied with a pipette into 24 well plates and mixed with a sucrose solution. The plates are closed with a stretched Parafilm. A plastic stencil with 24 holes is placed onto the plate and infested pea seedlings are placed directly on the Parafilm. The infested plate is closed with a gel blotting paper and another plastic stencil, and then turned upside down. 5 days after infestation the samples are checked for mortality.

In this test, compounds listed in the tables above show good activity. In particular compounds T14, T22, T61, T62 and T63 show an activity of over 80% at a concentration of 25 ppm.

1. A method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I): 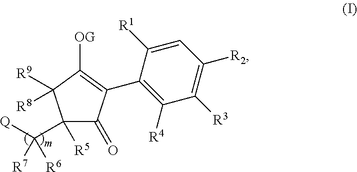
wherein
R¹ is hydrogen, methyl, ethyl, n-propyl, iso-propyl, halomethyl, haloethyl, halogen, vinyl, ethynyl, methoxy, ethoxy, halomethoxy, haloethoxy, cyclopropyl or halocyclopropyl,
R² and R³ are independently hydrogen, halogen, C₁-C₆alkyl, C₁-C₆haloalkyl, C₁-C₆alkoxy, C₁-C₆haloalkoxy, C₂-C₆alkenyl, C₂-C₆haloalkenyl, C₂-C₆alkynyl, C₂-C₆haloalkynyl, C₃-C₆alkenyloxy, C₃-C₆haloalkenyloxy, C₃-C₆alkynyloxy, C₃-C₆cycloalkyl, C₁-C₆alkylthio, C₁-C₆alkylsulfinyl, C₁-C₆alkylsulfonyl, C₁-C₆alkoxysulfonyl, C₁-C₆haloalkoxysulfonyl, cyano, nitro, phenyl, phenyl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, or heteroaryl or heteroaryl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl,
R⁴ is hydrogen, C₁-C₆alkyl, C₁-C₆haloalkyl, C₁-C₆alkoxy, C₁-C₆haloalkoxy, C₂-C₆alkenyl, C₂-C₆haloalkenyl, C₂-C₆alkynyl, C₃-C₆alkenyloxy, C₃-C₆haloalkenyloxy, C₃-C₆alkynyloxy, C₃-C₆cycloalkyl, C₁-C₆alkylthio, C₁-C₆alkylsulfinyl, C₁-C₆alkylsulfonyl, C₁-C₆alkoxysulfonyl, C₁-C₆haloalkoxysulfonyl or cyano,
R⁵, R⁶, R⁷, R⁸ and R⁹ are independently hydrogen, halogen, C₁-C₆alkyl, C₁-C₆haloalkyl, C₁-C₆alkoxy, C₁-C₆haloalkoxy, C₂-C₆alkenyl, C₂-C₆haloalkenyl, C₂-C₆alkynyl, C₃-C₆alkenyloxy, C₃-C₆haloalkenyloxy, C₃-C₆alkynyloxy, C₃-C₆cycloalkyl, C₁-C₆alkylthio, C₁-C₆alkylsulfinyl, C₁-C₆alkylsulfonyl, C₁-C₆haloalkylsulfonyl, C₁-C₆alkoxysulfonyl, C₁-C₆haloalkoxysulfonyl, cyano, nitro, phenyl, phenyl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, or heteroaryl or heteroaryl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl, C₁-C₃alkylsulfonyl, or benzyl or benzyl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, or C₃-C₆cycloalkylC₁-C₃alkyl in which a ring or chain methylene group is optionally replaced by an oxygen or sulfur atom or
R⁶ and R⁷ or R⁸ and R⁹ together with the carbon atoms to which they are attached form an optionally substituted 3- to 8-membered ring, optionally containing an oxygen, sulphur or nitrogen atom, or
R⁵ and R⁶ together form a bond,
Q is C₃-C₈ saturated or mono-unsaturated heterocyclyl containing at least one heteroatom selected from O, N and S, unsubstituted or substituted by a residue of formula ═O, ═N—R¹⁰ or C₁-C₄alkyl, C₁-C₄haloalkyl, C₁-C₄alkoxyC₁-C₂alkyl, C₃-C₆cycloalkyl, phenyl, phenyl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, where R¹⁰ is C₁-C₆alkyl, C₁-C₆haloalkyl, C₃-C₇cycloalkyl, C₁-C₆alkoxy, C₁-C₆haloalkoxy, C₁-C₆alkylsulfinyl, C₁-C₆alkylsulfonyl, C₁-C₆alkylcarbonyl, C₁-C₆haloalkylcarbonyl, C₁-C₆alkoxycarbonyl, C₁-C₆alkylaminocarbonyl, C₂-C₈dialkylaminocarbonyl, C₁-C₆haloalkylsulfinyl or C₁-C₆haloalkylsulfonyl,
m is 1, 2 or 3,
where R⁶ or R⁷ can have different meanings when m is 2 or 3, and
G is hydrogen or an agriculturally acceptable metal, sulfonium, ammonium or latentiating group.

2. A method according to claim 1, wherein R¹ is methyl, ethyl or methoxy.

3. A method according to claim 1 or claim 2, wherein R² and R³ are independently hydrogen, halogen, C₁-C₆alkyl, C₁-C₆alkoxy, C₂-C₆alkenyl, C₂-C₆alkynyl, C₂-C₆haloalkynyl, phenyl or phenyl substituted by C₁-C₄alkyl, C₁-C₃haloalkyl, cyano, nitro, halogen or C₁-C₃alkylsulfonyl.

4. A method according to claim 1 or claim 2, wherein R² and R³ are independently thienyl, thienyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, furyl, furyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, pyrazolyl, pyrazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, thiazolyl, thiazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, oxazolyl, oxazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, isothiazolyl, isothiazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, isoxazolyl, isoxazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, triazolyl, triazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, oxadiazolyl, oxadiazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, thiadiazolyl, thiadiazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, tetrazolyl, tetrazolyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, pyridyl, pyridyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, pyrimidinyl, pyrimidinyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, pyridazinyl, pyridazinyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, pyrazinyl or pyrazinyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, triazinyl or triazinyl substituted by C₁-C₃alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl.

5. A method according to claim 1 or claim 2, wherein R³ is hydrogen.

6. A method according to any preceding claim, wherein R⁴ is hydrogen, methyl, ethyl, n-propyl, iso-propyl, halomethyl, haloethyl, halogen, vinyl, ethynyl, methoxy, ethoxy, halomethoxy or haloethoxy.

7. A method according to any preceding claim, wherein R⁵ is hydrogen, halogen, C₁-C₆alkyl, C₁-C₆haloalkyl, C₁-C₆alkoxy or C₁-C₆haloalkoxy.

8. A method according to any preceding claim, wherein R⁶ and R⁷ independently are hydrogen, halogen, C₁-C₆alkyl, C₁-C₆haloalkyl, C₁-C₆alkoxy or C₁-C₆haloalkoxy.

9. A method according to any preceding claim, wherein R⁸ and R⁹ independently are hydrogen, halogen, C₁-C₆alkyl, C₁-C₆haloalkyl, C₁-C₆alkoxy or C₁-C₆haloalkoxy.

10. A method according to any preceding claim, wherein Q are those of the formula 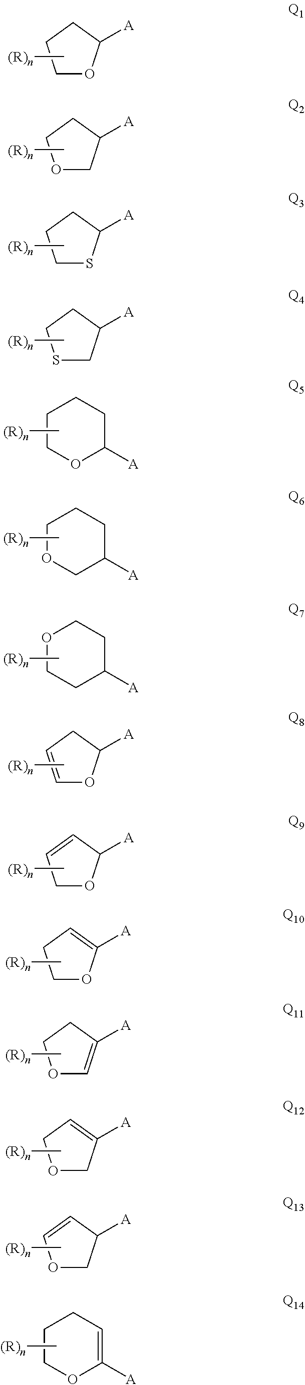 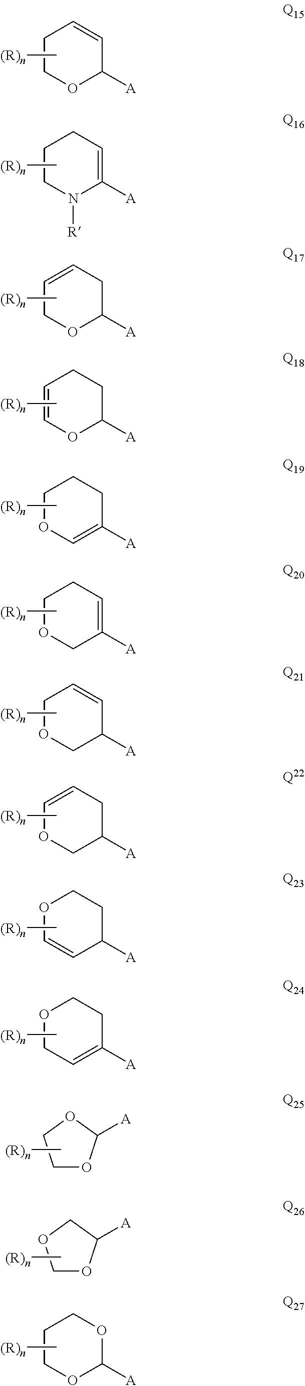 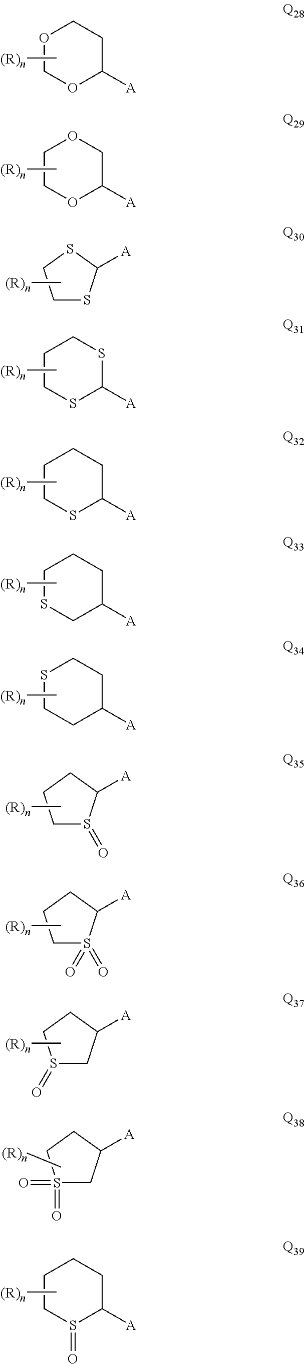 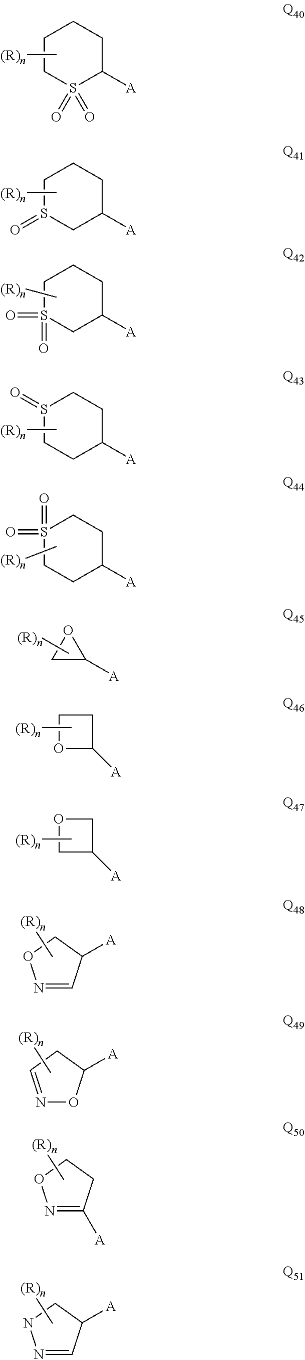 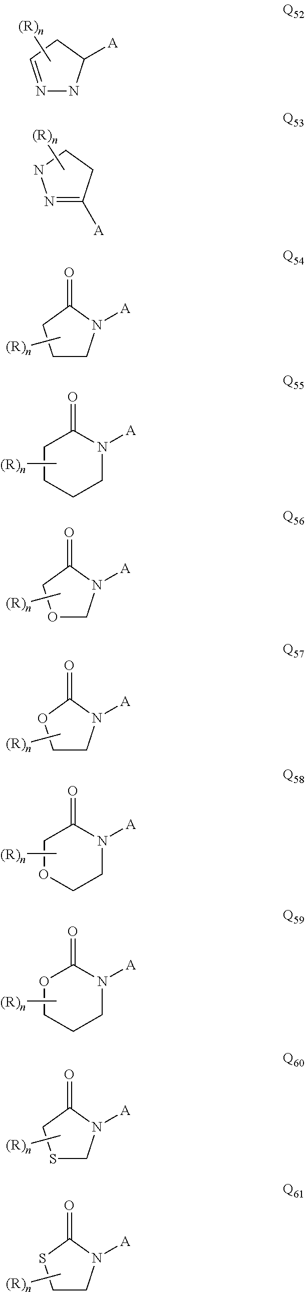 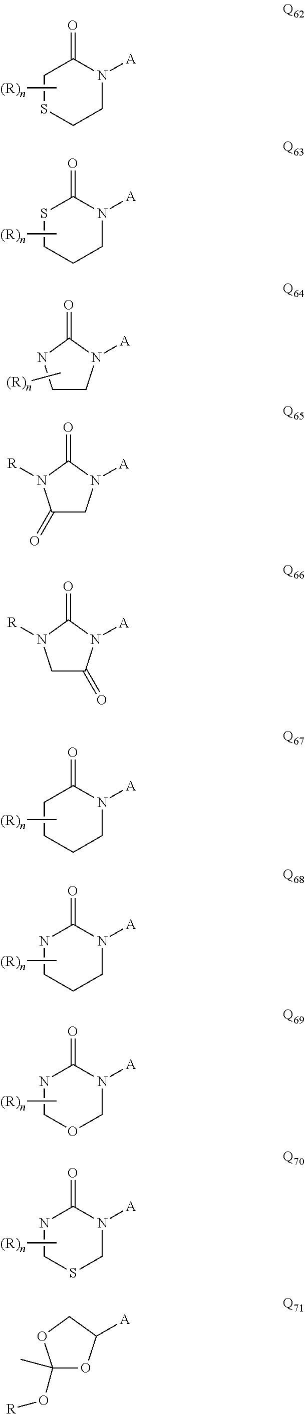 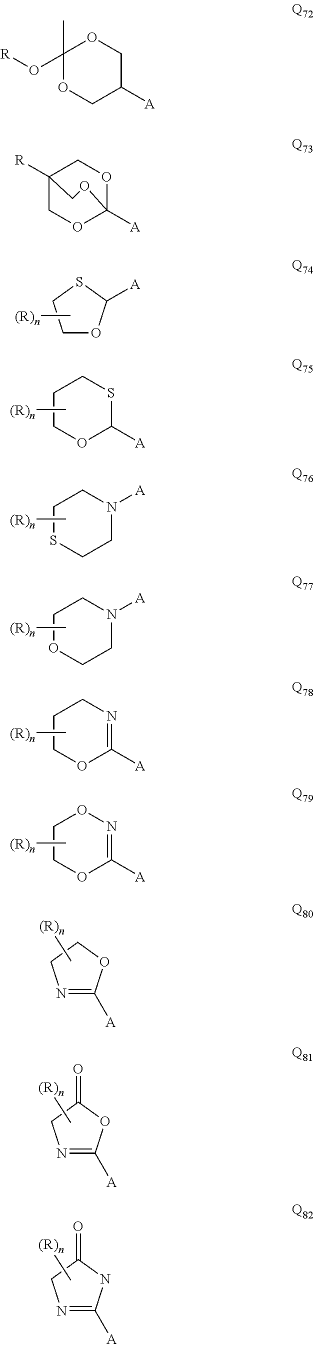 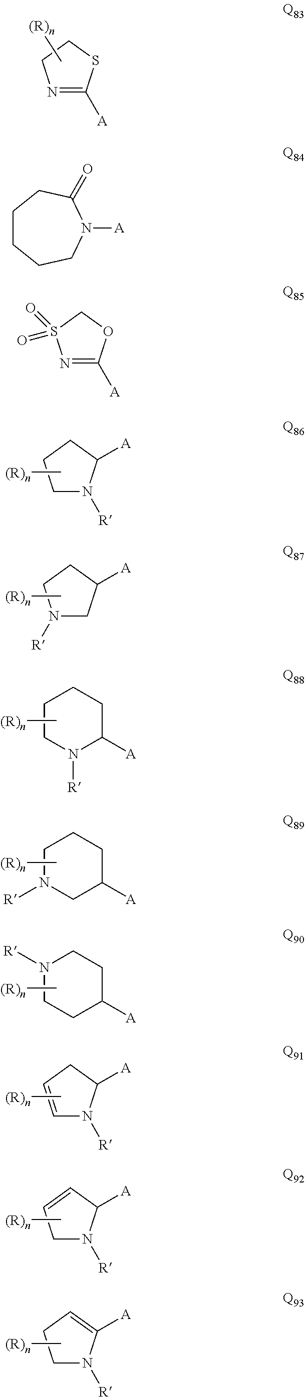 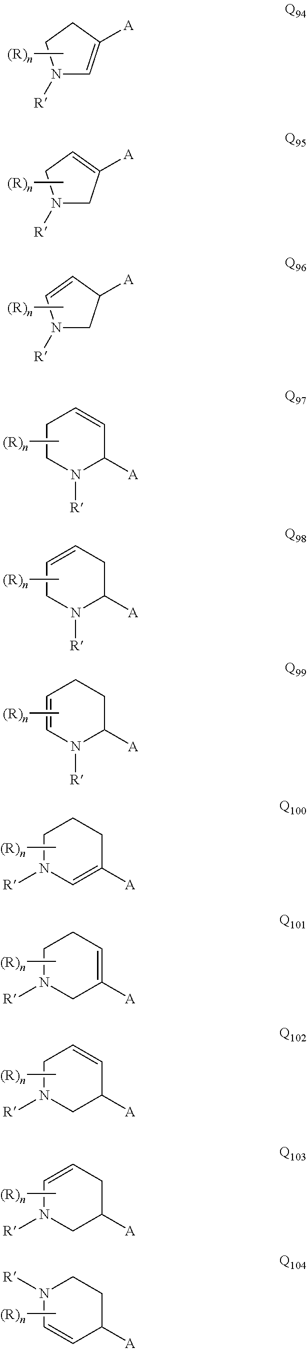 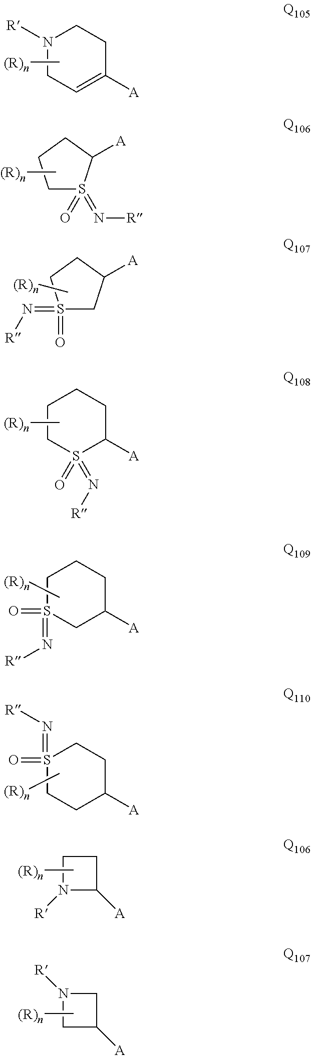
wherein
R is hydrogen, halogen, C₁-C₆alkyl, C₁-C₆haloalkyl, C₁-C₆alkoxy, C₁-C₆haloalkoxy, C₂-C₆alkenyl, C₂-C₆haloalkenyl, C₂-C₆alkynyl, C₂-C₆haloalkynyl, C₃-C₆alkenyloxy, C₃-C₆haloalkenyloxy, C₃-C₆alkynyloxy, C₃-C₆cycloalkyl, C₁-C₆alkylthio, C₁-C₆alkylsulfinyl, C₁-C₆alkylsulfonyl, C₁-C₆alkoxysulfonyl, C₁-C₆haloalkoxysulfonyl, cyano, nitro, phenyl, phenyl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl, or heteroaryl or heteroaryl substituted by C₁-C₄alkyl, C₁-C₃haloalkyl, C₁-C₃alkoxy, C₁-C₃haloalkoxy, cyano, nitro, halogen, C₁-C₃alkylthio, C₁-C₃alkylsulfinyl or C₁-C₃alkylsulfonyl,
R′ is hydrogen, C₁-C₆alkyl, C₁-C₆haloalkyl, C₃-C₇cycloalkyl, C₁-C₆alkoxy, C₁-C₆haloalkoxy, C₁-C₆alkylsulfinyl, C₁-C₆alkylsulfonyl, C₁-C₆alkylcarbonyl, C₁-C₆haloalkylcarbonyl, C₁-C₆alkoxycarbonyl, C₁-C₆alkylaminocarbonyl, C₂-C₈dialkylaminocarbonyl, C₆-C₁₀arylsulfonyl, C₆-C₁₀arylcarbonyl, C₆-C₁₀arylaminocarbonyl, C₇-C₁₆arylalkylaminocarbonyl, C₁-C₉hetarylsulfonyl, C₁-C₉hetarylcarbonyl, C₁-C₉hetarylaminocarbonyl, C₂-C₁₅hetarylalkylaminocarbonyl,
R″ is hydrogen, C₁-C₆alkyl, C₁-C₆haloalkyl, C₃-C₇cycloalkyl, C₁-C₆alkoxy, C₁-C₆haloalkoxy, C₁-C₆alkylsulfinyl, C₁-C₆alkylsulfonyl, C₁-C₆alkylcarbonyl, C₁-C₆haloalkylcarbonyl, C₁-C₆alkoxycarbonyl, C₁-C₆alkylaminocarbonyl, C₂-C₈dialkylaminocarbonyl, C₁-C₆haloalkylsulfinyl or C₁-C₆haloalkylsulfonyl,
n is 0, 1, 2, 3 and 4, and
A denotes the position of attachment to the —CR⁶R⁷— moiety.

11. An insecticidal, acaricidal and nematicidal composition comprising an insecticidally, acaricidally or nematicidally effective amount of a compound of formula I as defined in claim 1.

12. A pesticidal composition according to claim 11 comprising an effective amount of at least one insecticidally, acaricidally, nemacitidally or molluscicidally effective amount of a compound according to claim 1.

13. A method of combating and controlling pests which comprises applying to a pest, to a locus of a pest, or to a plant susceptible to attack by a pest a pesticidally effective amount of a compound of formula I.

14. A method according to claim 13 for the protection of plant propagation material from the attack by pests, which comprises applying to the propagation material or the site, where the propagation material is planted, a pesticidally effective amount of a compound of formula I.

15. A method according to claim 13 of combating and controlling insects, acarines, nematodes or molluscs, which comprises applying to the pest, to a locus of the pest, or to a plant susceptible to attack by the pest an insecticidally, acaricidally, nemacitidally or molluscicidally effective amount of a compound according to claim 1.
